(19) United States
(12) Patent Application Publication
(10) Pub. No.: US 2024/0174997 A1
(43) Pub. Date: May 30, 2024

(54) COMPOSITIONS COMPRISING A VARIANT POLYPEPTIDE AND USES THEREOF

(71) Applicant: Arbor Biotechnologies, Inc., Cambridge, MA (US)

(72) Inventors: Shaorong Chong, Arlington, MA (US); Brendan Jay Hilbert, Natick, MA (US); Quinton Norman Wessells, Cambridge, MA (US); Lauren E. Alfonse, Cambridge, MA (US); Anthony James Garrity, Hingham, MA (US)

(21) Appl. No.: 18/281,123

(22) PCT Filed: Mar. 9, 2022
(86) PCT No.: PCT/US22/19536

§ 371 Date: Sep. 8, 2023

Related U.S. Application Data
(60) Provisional application No. 63/294,224, filed on Dec. 28, 2021.
(60) Provisional application No. 63/158,741, filed on Mar. 9, 2021.

(51) Int. Cl. C12N 9/22; C12N 15/10; C12N 15/11; C12N 15/86

(52) U.S. Cl. CPC C12N 9/22; C12N 15/102; C12N 15/11; C12N 15/86; C07K 2319/09; C12N 2310/20

(57) ABSTRACT

The present invention relates to variant polypeptides, methods of preparing the variant polypeptides, processes for characterizing the variant polypeptides, compositions and cells comprising the variant polypeptides, and methods of using the variant polypeptides. The invention further relates to complexes comprising the variant polypeptides, methods of producing the complexes, processes for characterizing the complexes, cells comprising the complexes, and methods of using the complexes.

### RELATED APPLICATIONS

This application claims priority to U.S. Provisional Application 63/158,741 filed on Mar. 9, 2021, U.S. Provisional Application 63/294,224 filed on Dec. 28, 2021, the entire contents of each of which are hereby incorporated by reference.

### SEQUENCE LISTING

The instant application contains a Sequence Listing which has been submitted electronically in ASCII format and is hereby incorporated by reference in its entirety. Said ASCII copy, created on Mar. 7, 2022 is named A2186-7046WO_SL.txt and is 38,750 bytes in size.

### BACKGROUND

Clustered Regularly Interspaced Short Palindromic Repeats (CRISPR) and CRISPR-associated (Cas) genes, collectively known as CRISPR-Cas or CRISPR/Cas systems, are adaptive immune systems in archaea and bacteria that defend particular species against foreign genetic elements.

### SUMMARY OF THE INVENTION

It is against the above background that the present invention provides certain advantages and advancements over the prior art.

Although this invention disclosed herein is not limited to specific advantages or functionalities, the invention provides a variant polypeptide comprising an alteration relative to a parent polypeptide of SEQ ID NO: 3, and wherein the alteration is a substitution of Table 2. In some embodiments, the substitution is a P14R substitution, an E311R substitution, a D32R substitution, an 161R substitution, a G223R substitution, an N109R substitution, and/or a D719R substitution.

In certain embodiments, the variant polypeptide comprises a) a P14R substitution, an E311R substitution, and a D32R substitution: b) a P14R substitution, an E311R substitution, and a G223R substitution; c) a P14R substitution, an E311R substitution, a D32R substitution, and an 161R substitution; or d) a D32R substitution, an N109R substitution, an E311R substitution, and a D719R substitution. In some embodiments, the variant polypeptide comprises c) a P14R substitution, an E311R substitution, a D32R substitution, and an 161R substitution.

In some embodiments, the variant polypeptide further comprises a K208G substitution, a D302G substitution, a D590G substitution, an E154G substitution, a D567G substitution, an L38G substitution, a D145G substitution, a C13G substitution, a T338G substitution, a P14G substitution, a D55G substitution, a K221G substitution, a K35G substitution, and an E736G substitution.

In one aspect, the disclosure provides a variant polypeptide comprising an amino acid sequence having at least 95% identity to SEQ ID NO: 3 and comprising a substitution one or more of positions P14, E311, D32, 161, G223, N109, and D719 relative to SEQ ID NO: 3.

In one aspect, the disclosure provides a variant polypeptide comprising an amino acid sequence having at least 95% identity to SEQ ID NO: 3 and comprising one or more of the following substitutions: P14R, E311R, D32R, 161R, G223R, N109R, and D719R.

In one aspect, the disclosure provides a variant polypeptide comprising an amino acid sequence having at least 95% identity to SEQ ID NO: 3 and comprising a substitution one or more of positions K208, D302, D590, E154, D567, L38, D145, C13, T338, P14, D55, K221, K35, and E736 relative to SEQ ID NO: 3.

In one aspect, the disclosure provides a variant polypeptide comprising an amino acid sequence having at least 95% identity to SEQ ID NO: 3 and comprising one or more of the following substitutions: K208G, D302G, D590G, E154G, D567G, L38G, D145G, C13G, T338G, P14G, D55G, K221G, K35G, and E736G.

In some embodiments, the variant polypeptide comprises a substitution at P14 (e.g., a P14R substitution), In certain embodiments, the variant polypeptide comprises a substitution at E311 (e.g., an E311R substitution), In some embodiments, the variant polypeptide comprises a substitution at D32 (e.g., a D32R substitution), In certain embodiments, the variant polypeptide comprises a substitution at 161 (e.g., a I61R substitution), In some embodiments, the variant polypeptide comprises a substitution at G223 (e.g., a G223 substitution), In certain embodiments, the variant polypeptide comprises a substitution at N109 (e.g., a N109R substitution), In some embodiments, the variant polypeptide comprises a substitution at D719 (e.g., a D719R substitution),

In certain embodiments, the variant polypeptide comprises a substitution at position P14 (e.g., a P14R substitution), an E311 (e.g., an E311R substitution), a D32 (e.g., a D32R substitution), an 161 (e.g., a 161R substitution), a G223 (e.g., a G223 substitution), an N109 (e.g., a N109R substitution), a D719 (e.g., a D719R substitution), or any combination thereof.

In some embodiments, the variant polypeptide comprises a substitution at position P14 (e.g., a P14R substitution), E311 (e.g., an E311R), and D32 (e.g., a D32R substitution) relative to SEQ ID NO: 3 (e.g., a P14R, E311R, D32R variant polypeptide).

In some embodiments, the variant polypeptide comprises a substitution at position P14 (e.g., a P14R substitution), E311 (e.g., an E311R), and G223 (e.g., a G223R substitution) relative to SEQ ID NO: 3 (e.g., a P14R, E311R, G223R variant polypeptide).

In certain embodiments, the variant polypeptide comprises a substitution at position P14 (e.g., a P14R substitution), E311 (e.g., an E311R), D32 (e.g., a D32R substitution), and 161 (e.g., an I61R substitution) relative to SEQ ID NO: 3 (e.g., a P14R, E311R, D32R and I61R variant polypeptide).

In some embodiments, the variant polypeptide comprises a substitution at position D32R (e.g., a D32R substitution), N109 (e.g., an N109R), E311 (e.g., an E311R substitution), and D719 (e.g., a D719R substitution) relative to SEQ ID NO: 3 (e.g., a D32R, N109R, E311R and D719R variant polypeptide).

In certain embodiments, the variant polypeptide comprises a substitution at K208 (e.g., a K208G substitution), D302 (e.g., a D302G substitution), D590 (e.g., a D590G substitution), E154 (e.g., an E154G substitution), D567 (e.g., a D567G substitution), L38 (e.g., an L38G substitution), D145 (e.g., a D145G substitution), C13 (e.g., a C13G substitution), T338 (e.g., a T338G substitution), P14 (e.g., a P14G substitution), D55 (e.g., a D55G substitution), K221 (e.g., a K221G substitution), K35 (e.g., a K35G substitution), and E736 (e.g., an E736G substitution), or any combination thereof.

In particular embodiments, the variant polypeptide exhibits increased binary complex formation with an RNA guide, relative to a parent polypeptide. In certain embodiments, a binary complex comprising the variant polypeptide exhibits increased stability, relative to a parent binary complex.

In some embodiments, the variant polypeptide exhibits increased nuclease activity, relative to a parent polypeptide.

In one aspect, the disclosure provides a composition comprising the variant polypeptide described herein, wherein the composition further comprises an RNA guide or a nucleic acid encoding the RNA guide, wherein the RNA guide comprises a direct repeat sequence and a spacer sequence. In certain embodiments, the direct repeat sequence is at least 90% (e.g., 90%, 91%, 92%, 93%, 94%, 95%, 96%, 97%, 98%, 99% or 100%) identical to any one of SEQ ID NOs: 4-13 or comprises a sequence having at least 90% (e.g., 90%, 91%, 92%, 93%, 94%, 95%, 96%, 97%, 98%, 99% or 100%) identity to SEQ ID NO: 14 or SEQ ID NO: 15. In some embodiments, the direct repeat sequence is at least 95% (e.g., 95%, 96%, 97%, 98%, 99% or 100%) identical to any one of SEQ ID NOs: 4-13 or comprises a sequence having at least 95% (e.g., 95%, 96%, 97%, 98%, 99% or 100%) identity to SEQ ID NO: 14 or SEQ ID NO: 15. In some embodiments, the direct repeat sequence is any one of SEQ ID NOs: 4-13 or comprises a sequence of SEQ ID NO: 14 or SEQ ID NO: 15.

In some embodiments, the spacer sequence comprises about 15 nucleotides to about 35 nucleotides in length.

In certain embodiments, the spacer sequence binds to a target strand sequence of a target nucleic acid, and wherein a non-target strand sequence of the target nucleic acid sequence is adjacent to a protospacer adjacent motif (PAM) sequence. In some embodiments, the PAM sequence is 5′-TTR-3′, 5′-NTTR-3′, 5′-NTTN-3′, 5′-RTTR-3′, 5′-ATTR-3′, or 5′-RTTG-3′, wherein N is any nucleotide, Y is C or T, and R is A or G. In certain embodiments, the PAM sequence is 5′-TTG-3′, 5′-TTA-3′, 5′-ATTG-3″, 5′-TTTA-3′, or 5′-TTTG-3″.

In certain embodiments, the variant polypeptide further comprises a nuclear localization signal (NLS).

In some embodiments, the variant polypeptide further comprises a peptide tag, a fluorescent protein, a base-editing domain, a DNA methylation domain, a histone residue modification domain, a localization factor, a transcription modification factor, a light-gated control factor, a chemically inducible factor, or a chromatin visualization factor.

In one aspect, the disclosure provides a composition comprising a nucleic acid that encodes the variant polypeptide and/or the RNA guide described anywhere herein. In some embodiments, the nucleic acid is codon-optimized for expression in a cell. In certain embodiments, the nucleic acid is operably linked to a promoter. In some embodiments, the nucleic acid is in a vector. In certain embodiments, the vector comprises a retroviral vector, a lentiviral vector, a phage vector, an adenoviral vector, an adeno-associated vector, or a herpes simplex vector.

In some embodiments, the variant polypeptide is present in a delivery system comprising a nanoparticle (e.g., a lipid nanoparticle), a liposome, an exosome, a microvesicle, or a gene-gun.

In one aspect, the disclosure provides a cell comprising the variant polypeptide or the composition of any previous aspect or embodiment.

In some embodiments, the cell is a eukaryotic cell. In certain embodiments, the cell is a mammalian cell or a plant cell. In certain embodiments, the cell is a human cell.

In one aspect, the disclosure provides a composition comprising a variant polypeptide or a complex comprising the variant polypeptide, wherein the variant polypeptide comprises an alteration relative to a parent polypeptide of SEQ ID NO: 3, and wherein the variant polypeptide or the complex exhibits enhanced enzymatic activity, enhanced binding activity, enhanced binding specificity, and/or enhanced stability, relative to a parent polypeptide or a complex comprising the parent polypeptide.

In some embodiments, the alteration is a substitution of Table 2.

In certain embodiments, the substitution is a P14R substitution, an E311R substitution, a D32R substitution, an I61R substitution, a G223R substitution, an N109R substitution, and/or a D719R substitution.

In some embodiments, the variant polypeptide comprises a) a P14R substitution, an E311R substitution, and a D32R substitution: b) a P14R substitution, an E311R substitution, and a G223R substitution: c) a P14R substitution, an E311R substitution, a D32R substitution, and an I61R substitution; or d) a D32R substitution, an N109R substitution, an E311R substitution, and a D719R substitution.

In certain embodiments, the variant polypeptide further comprises a K208G substitution, a D302G substitution, a D590G substitution, an E154G substitution, a D567G substitution, an L38G substitution, a D145G substitution, a C13G substitution, a T338G substitution, a P14G substitution, a D55G substitution, a K221G substitution, a K35G substitution, and an E736G substitution.

In some embodiments, the enhanced enzymatic activity is enhanced nuclease activity.

In certain embodiments, the variant polypeptide exhibits enhanced binding activity to an RNA guide, relative to the parent polypeptide.

In some embodiments, the variant polypeptide exhibits enhanced binding specificity to an RNA guide, relative to the parent polypeptide.

In some embodiments, the complex comprising the variant polypeptide is a variant binary complex that further comprises an RNA guide, and the variant binary complex exhibits enhanced binding activity to a target nucleic acid (e.g., on-target binding activity), relative to a parent binary complex.

In still another embodiment, the complex comprising the variant polypeptide is a variant binary complex that further comprises an RNA guide, and the variant binary complex exhibits enhanced binding specificity to a target nucleic acid (e.g., on-target binding specificity), relative to a parent binary complex.

In some embodiments, the complex comprising the variant polypeptide is a variant binary complex that further comprises an RNA guide, and the variant binary complex exhibits enhanced stability, relative to a parent binary complex.

In certain embodiments, the variant binary complex and a target nucleic acid form a variant ternary complex, and the variant ternary complex exhibits increased stability, relative to a parent ternary complex.

In some embodiments, the variant polypeptide further exhibits enhanced binary complex formation, enhanced protein-RNA interactions, and/or decreased dissociation from an RNA guide, relative to the parent polypeptide.

In certain embodiments, the variant binary complex further exhibits decreased dissociation from a target nucleic acid, and/or decreased off-target binding to a non-target nucleic acid, relative to the parent binary complex.

In some embodiments, the enhanced enzymatic activity, enhanced binding activity, enhanced binding specificity, and/or enhanced stability occur over a range of temperatures, e.g., 20° C., to 65° C. (e.g., 20° C., to 30° C., 30° C., to 40° C., 40° C., to 50° C., 50° C., to 60° C., or 60° C., to 65°) C.

In certain embodiments, the enhanced enzymatic activity, enhanced binding activity, enhanced binding specificity, and/or enhanced stability occur over a range of incubation times.

In some embodiments, the enhanced enzymatic activity, enhanced binding activity, enhanced binding specificity, and/or enhanced stability occur in a buffer having a pH in a range of about 7.3 to about 8.6 (e.g., about 7.5 to about 8.0, about 7.8 to 8.3, or about 8.0 to 8.6).

In certain embodiments, the enhanced enzymatic activity, enhanced binding activity, enhanced binding specificity, and/or enhanced stability occurs when a Tₘ value of the variant polypeptide, variant binary complex, or variant ternary complex is at least 8° C. greater than the Tₘ value of the parent polypeptide, parent binary complex, or parent ternary complex.

In some embodiments, the variant polypeptide comprises a RuvC domain or a split RuvC domain.

In certain embodiments, the parent polypeptide comprises the sequence of SEQ ID NO: 3.

In some embodiments, the RNA guide comprises a direct repeat sequence and a spacer sequence.

In some embodiments, the direct repeat sequence is at least 90% (e.g., 90%, 91%, 92%, 93%, 94%, 95%, 96%, 97%, 98%, 99% or 100%) identical to any one of SEQ ID NOs: 4-13 or comprises a sequence having at least 90% (e.g., 90%, 91%, 92%, 93%, 94%, 95%, 96%, 97%, 98%, 99% or 100%) identity to SEQ ID NO: 14 or SEQ ID NO: 15.

In some embodiments, the direct repeat sequence is at least 95% (e.g., 95%, 96%, 97%, 98%, 99% or 100%) identical to any one of SEQ ID NOs: 4-13 or comprises a sequence having at least 95% (e.g., 95%, 96%, 97%, 98%, 99% or 100%) identity to SEQ ID NO: 14 or SEQ ID NO: 15. In some embodiments, the direct repeat sequence is any one of SEQ ID NOs: 4-13 or comprises a sequence of SEQ ID NO: 14 or SEQ ID NO: 15.

In certain embodiments, the spacer sequence comprises between 15 and 35 nucleotides in length.

In certain embodiments, the spacer sequence comprises complementarity to a target strand sequence of a target nucleic acid.

In some embodiments, the target nucleic acid comprises a non-target strand sequence adjacent to a protospacer adjacent motif (PAM) sequence. In certain embodiments, the PAM sequence is 5′-TTR-3′, 5′-NTTR-3′, 5′-NTTN-3′, 5′-RTTR-3′, 5′-ATTR-3′, or 5′-RTTG-3′, wherein N is any nucleotide, Y is C or T, and R is A or G. In some embodiments, the PAM sequence is 5′-TTG-3′, 5′-TTA-3′, 5′-ATTG-3′, 5′-TTTA-3′, or 5′-TTTG-3′.

In certain embodiments, the variant polypeptide further comprises a peptide tag, a fluorescent protein, a base-editing domain, a DNA methylation domain, a histone residue modification domain, a localization factor, a transcription modification factor, a light-gated control factor, a chemically inducible factor, or a chromatin visualization factor.

In one aspect, the disclosure provides a composition comprising a nucleic acid that encodes the variant polypeptide of the previous aspect or embodiments thereof, wherein optionally the nucleic acid is codon-optimized for expression in a cell.

In some embodiments, the cell is a eukaryotic cell.

In some embodiments, the cell is a mammalian cell or a plant cell. In certain embodiments, the cell is a human cell.

In some embodiments, the nucleic acid encoding the variant polypeptide is operably linked to a promoter.

In certain embodiments, the nucleic acid encoding the variant polypeptide is in a vector.

In some embodiments, the vector comprises a retroviral vector, a lentiviral vector, a phage vector, an adenoviral vector, an adeno-associated vector, or a herpes simplex vector.

In certain embodiments, the composition is present in a delivery composition comprising a nanoparticle (e.g., a lipid nanoparticle), a liposome, an exosome, a microvesicle, or a gene-gun.

In one aspect, the disclosure provides a method for editing a gene in a cell, the method comprising contacting the cell with the variant polypeptide or composition of any one of the previous aspects or embodiments.

In one aspect, the disclosure provides a nucleic acid molecule encoding a variant polypeptide of any of the previous aspects of embodiments.

In certain embodiments, the sequence of the nucleic acid molecule is 95% identical to a sequence selected from the group consisting of SEQ ID NOs: 1, 2, or 21-27. In some embodiments, the sequence of the nucleic acid molecule comprises a sequence selected from the group consisting of SEQ ID NOs: 1, 2, or 21-27. In certain embodiments, the sequence of the nucleic acid molecule is 95% identical to a sequence selected from the group consisting of SEQ ID NOs: 22, 23, or 25.

Although this invention disclosed herein is not limited to specific advantages or functionalities, the invention provides a variant polypeptide, and/or a composition comprising a variant polypeptide, wherein the variant polypeptide comprises an alteration relative to the parent polypeptide of SEQ ID NO: 3, and wherein the variant polypeptide or a complex comprising the variant polypeptide exhibits enhanced enzymatic activity, enhanced binding activity, enhanced binding specificity, and/or enhanced stability relative to the parent polypeptide or a complex comprising the parent polypeptide.

In some aspects, the enhanced enzymatic activity is enhanced nuclease activity.

In some aspects, the variant polypeptide exhibits enhanced binding activity to an RNA guide relative to the parent polypeptide.

In some aspects, the variant polypeptide exhibits enhanced binding specificity to an RNA guide relative to the parent polypeptide.

In some aspects, the variant polypeptide and an RNA guide form a variant binary complex, and the variant binary complex exhibits enhanced binding activity to a target nucleic acid (e.g., on-target binding activity) relative to a parent binary complex.

In some aspects, the variant polypeptide and an RNA guide form a variant binary complex, and the variant binary complex exhibits enhanced binding specificity to a target nucleic acid (e.g., on-target binding specificity) relative to a parent binary complex.

In some aspects, the variant polypeptide and an RNA guide form a variant binary complex, and the variant binary complex exhibits enhanced stability relative to a parent binary complex.

In some aspects, the variant binary complex and a target nucleic acid form a variant ternary complex, and the variant ternary complex exhibits increased stability relative to a parent ternary complex.

In some aspects, the variant polypeptide further exhibits enhanced binary complex formation, enhanced protein-RNA interactions, and/or decreased dissociation from an RNA guide relative to the parent polypeptide.

In some aspects, the variant binary complex further exhibits decreased dissociation from the target nucleic acid, and/or decreased off-target binding to a non-target nucleic acid relative to the parent binary complex.

In some aspects, the enhanced enzymatic activity, enhanced binding activity, enhanced binding specificity, and/or enhanced stability occur over a range of temperatures, e.g., 20° C., to 65° C. (e.g., 20° C., to 30° C., 30° C., to 40° C., 40° C., to 50° C., 50° C., to 60° C., or 60° C., to 65°) C.

In some aspects, the enhanced enzymatic activity, enhanced binding activity, enhanced binding specificity, and/or enhanced stability occur over a range of incubation times.

In some aspects, the enhanced enzymatic activity, enhanced binding activity, enhanced binding specificity, and/or enhanced stability occur in a buffer having a pH in a range of about 7.3 to about 8.6 (e.g., about 7.5 to about 8.0, about 7.8 to 8.3, or about 8.0 to 8.6).

In some aspects, the enhanced enzymatic activity, enhanced binding activity, enhanced binding specificity, and/or enhanced stability occurs when a Tₘ value of the variant polypeptide, variant binary complex, or variant ternary complex is at least 8° C. greater than the Tₘ value of the parent polypeptide, parent binary complex, or parent ternary complex.

In other aspects, the alteration comprises an amino acid sequence alteration relative to the parent polypeptide having the sequence set forth in SEQ ID NO: 3, wherein the alteration comprises one or more (e.g., one, two, three, four, five, or more) substitutions, insertions, deletions, and/or additions as compared to the parent polypeptide having the sequence set forth in SEQ ID NO:3.

In some aspects, the alteration comprises an amino acid sequence alteration relative to the parent polypeptide sequence set forth in SEQ ID NO: 3, wherein the alteration comprises one or more of the amino acid substitutions listed in Table 2.

In some aspects, the alteration comprises an arginine, lysine, glutamine, asparagine, histidine, alanine, or glycine substitution.

In some aspects, the alteration is an amino acid substitution selected from P14R, E311R, D32R, I61R, G223R, N109R, and/or D719R.

In some aspects, the present disclosure provides a polypeptide comprising an amino acid sequence having at least 95%, 96%, 97%, 98%, or 99% identity to SEQ ID NO: 3 and comprising a substitution at position P14 (e.g., a P14R substitution) relative to SEQ ID NO: 3. In some aspects, the present disclosure provides a polypeptide comprising an amino acid sequence having one or more sequence alterations (e.g., substitutions, insertions, or deletions, or any combination thereof) at up to 1, 2, 3, 4, 5, 10, 15, 20, 25, 30, or 35 amino acid positions of SEQ ID NO: 3, wherein one of the sequence alterations comprises a substitution at position P14 (e.g., a P14R substitution) relative to SEQ ID NO: 3.

In some aspects, the present disclosure provides a polypeptide comprising an amino acid sequence having at least 95%, 96%, 97%, 98%, or 99% identity to SEQ ID NO: 3 and comprising a substitution at position E311 (e.g., an E311R substitution) relative to SEQ ID NO: 3. In some aspects, the present disclosure provides a polypeptide comprising an amino acid sequence having one or more sequence alterations (e.g., substitutions, insertions, or deletions, or any combination thereof) at up to 1, 2, 3, 4, 5, 10, 15, 20, 25, 30, or 35 amino acid positions of SEQ ID NO: 3, wherein one of the sequence alterations comprises a substitution at position E311 (e.g., an E31IR substitution) relative to SEQ ID NO: 3.

In some aspects, the present disclosure provides a polypeptide comprising an amino acid sequence having at least 95%, 96%, 97%, 98%, or 99% identity to SEQ ID NO: 3 and comprising a substitution at position D32 (e.g., a D32R substitution) relative to SEQ ID NO: 3. In some aspects, the present disclosure provides a polypeptide comprising an amino acid sequence having one or more sequence alterations (e.g., substitutions, insertions, or deletions, or any combination thereof) at up to 1, 2, 3, 4, 5, 10, 15, 20, 25, 30, or 35 amino acid positions of SEQ ID NO: 3, wherein one of the sequence alterations comprises a substitution at position D32 (e.g., a D32R substitution) relative to SEQ ID NO: 3.

In some aspects, the present disclosure provides a polypeptide comprising an amino acid sequence having at least 95%, 96%, 97%, 98%, or 99% identity to SEQ ID NO: 3 and comprising a substitution at position 161 (e.g., an 161R substitution) relative to SEQ ID NO: 3. In some aspects, the present disclosure provides a polypeptide comprising an amino acid sequence having one or more sequence alterations (e.g., substitutions, insertions, or deletions, or any combination thereof) at up to 1, 2, 3, 4, 5, 10, 15, 20, 25, 30, or 35 amino acid positions of SEQ ID NO: 3, wherein one of the sequence alterations comprises a substitution at position 161 (e.g., an 161R substitution) relative to SEQ ID NO: 3.

In some aspects, the present disclosure provides a polypeptide comprising an amino acid sequence having at least 95%, 96%, 97%, 98%, or 99% identity to SEQ ID NO: 3 and comprising a substitution at position G223 (e.g., a G223R substitution) relative to SEQ ID NO: 3. In some aspects, the present disclosure provides a polypeptide comprising an amino acid sequence having one or more sequence alterations (e.g., substitutions, insertions, or deletions, or any combination thereof) at up to 1, 2, 3, 4, 5, 10, 15, 20, 25, 30, or 35 amino acid positions of SEQ ID NO: 3, wherein one of the sequence alterations comprises a substitution at position G223 (e.g., a G223R substitution) relative to SEQ ID NO: 3.

In some aspects, the present disclosure provides a polypeptide comprising an amino acid sequence having at least 95%, 96%, 97%, 98%, or 99% identity to SEQ ID NO: 3 and comprising a substitution at position N109 (e.g., an N109R substitution) relative to SEQ ID NO: 3. In some aspects, the present disclosure provides a polypeptide comprising an amino acid sequence having one or more sequence alterations (e.g., substitutions, insertions, or deletions, or any combination thereof) at up to 1, 2, 3, 4, 5, 10, 15, 20, 25, 30, or 35 amino acid positions of SEQ ID NO: 3, wherein one of the sequence alterations comprises a substitution at position N109 (e.g., an N109R substitution) relative to SEQ ID NO: 3.

In some aspects, the present disclosure provides a polypeptide comprising an amino acid sequence having at least 95%, 96%, 97%, 98%, or 99% identity to SEQ ID NO: 3 and comprising a substitution at position D719 (e.g., a D719R substitution) relative to SEQ ID NO: 3. In some aspects, the present disclosure provides a polypeptide comprising an amino acid sequence having one or more sequence alterations (e.g., substitutions, insertions, or deletions, or any combination thereof) at up to 1, 2, 3, 4, 5, 10, 15, 20, 25, 30, or 35 amino acid positions of SEQ ID NO: 3, wherein one of the sequence alterations comprises a substitution at position D719 (e.g., a D719R substitution) relative to SEQ ID NO: 3.

In some aspects, the alteration is a combination of amino acid substitutions listed in Table 3.

In some aspects, the combination of amino acid substitutions comprises the substitutions set forth in a) P14R, E311R, D32R: b) P14R, E311R, G223R: c) P14R, E311R, D32R, 161R: or d) D32R, N109R, E311R, D719R.

In some aspects, the present disclosure provides a polypeptide comprising an amino acid having at least 95%, 96%, 97%, 98%, or 99% identity to SEQ ID NO: 3 and comprising a substitution at position P14 (e.g., a P14R substitution), E311 (e.g., an E311R), and D32 (e.g., a D32R substitution) relative to SEQ ID NO: 3 (e.g., a P14R, E31IR, D32R variant polypeptide).

In some aspects, the present disclosure provides a polypeptide comprising an amino acid having at least 95%, 96%, 97%, 98%, or 99% identity to SEQ ID NO: 3 and comprising a substitution at position P14 (e.g., a P14R substitution), E311 (e.g., an E311R), and G223 (e.g., a G223R substitution) relative to SEQ ID NO: 3 (e.g., a P14R, E311R, G223R variant polypeptide).

In some aspects, the present disclosure provides a polypeptide comprising an amino acid having at least 95%, 96%, 97%, 98%, or 99% identity to SEQ ID NO: 3 and comprising a substitution at position P14 (e.g., a P14R substitution), E311 (e.g., an E311R), D32 (e.g., a D32R substitution), and 161 (e.g., an 161R substitution) relative to SEQ ID NO: 3 (e.g., a P14R, E311R, D32R and 161R variant polypeptide).

In some aspects, the present disclosure provides a polypeptide comprising an amino acid having at least 95%, 96%, 97%, 98%, or 99% identity to SEQ ID NO: 3 and comprising a substitution at position D32R (e.g., a D32R substitution), N109 (e.g., an N109R), E311 (e.g., an E31IR substitution), and D719 (e.g., a D719R substitution) relative to SEQ ID NO: 3 (e.g., a D32R, N109R, E311R and D719R variant polypeptide).

In some aspects, the present disclosure provides a polypeptide comprising an amino acid sequence having at least 95%, 96%, 97%, 98%, or 99% identity to SEQ ID NO: 3 and comprising a substitution at position K208 (e.g., a K208G substitution) relative to SEQ ID NO: 3. In some aspects, the present disclosure provides a polypeptide comprising an amino acid sequence having one or more sequence alterations (e.g., substitutions, insertions, or deletions, or any combination thereof) at up to 1, 2, 3, 4, 5, 10, 15, 20, 25, 30, or 35 amino acid positions of SEQ ID NO: 3, wherein one of the sequence alterations comprises a substitution at position K208 (e.g., a K208G substitution) relative to SEQ ID NO: 3.

In some aspects, the present disclosure provides a polypeptide comprising an amino acid sequence having at least 95%, 96%, 97%, 98%, or 99% identity to SEQ ID NO: 3 and comprising a substitution at position D302 (e.g., a D302G substitution) relative to SEQ ID NO: 3. In some aspects, the present disclosure provides a polypeptide comprising an amino acid sequence having one or more sequence alterations (e.g., substitutions, insertions, or deletions, or any combination thereof) at up to 1, 2, 3, 4, 5, 10, 15, 20, 25, 30, or 35 amino acid positions of SEQ ID NO: 3, wherein one of the sequence alterations comprises a substitution at position D302 (e.g., a D302G substitution) relative to SEQ ID NO: 3.

In some aspects, the present disclosure provides a polypeptide comprising an amino acid sequence having at least 95%, 96%, 97%, 98%, or 99% identity to SEQ ID NO: 3 and comprising a substitution at position D590 (e.g., a D590G substitution) relative to SEQ ID NO: 3. In some aspects, the present disclosure provides a polypeptide comprising an amino acid sequence having one or more sequence alterations (e.g., substitutions, insertions, or deletions, or any combination thereof) at up to 1, 2, 3, 4, 5, 10, 15, 20, 25, 30, or 35 amino acid positions of SEQ ID NO: 3, wherein one of the sequence alterations comprises a substitution at position D590 (e.g., a D590G substitution) relative to SEQ ID NO: 3.

In some aspects, the present disclosure provides a polypeptide comprising an amino acid sequence having at least 95%, 96%, 97%, 98%, or 99% identity to SEQ ID NO: 3 and comprising a substitution at position E154 (e.g., an E154G substitution) relative to SEQ ID NO: 3. In some aspects, the present disclosure provides a polypeptide comprising an amino acid sequence having one or more sequence alterations (e.g., substitutions, insertions, or deletions, or any combination thereof) at up to 1, 2, 3, 4, 5, 10, 15, 20, 25, 30, or 35 amino acid positions of SEQ ID NO: 3, wherein one of the sequence alterations comprises a substitution at position E154 (e.g., an E154G substitution) relative to SEQ ID NO: 3.

In some aspects, the present disclosure provides a polypeptide comprising an amino acid sequence having at least 95%, 96%, 97%, 98%, or 99% identity to SEQ ID NO: 3 and comprising a substitution at position D567 (e.g., a D567G substitution) relative to SEQ ID NO: 3. In some aspects, the present disclosure provides a polypeptide comprising an amino acid sequence having one or more sequence alterations (e.g., substitutions, insertions, or deletions, or any combination thereof) at up to 1, 2, 3, 4, 5, 10, 15, 20, 25, 30, or 35 amino acid positions of SEQ ID NO: 3, wherein one of the sequence alterations comprises a substitution at position D567 (e.g., a D567G substitution) relative to SEQ ID NO: 3.

In some aspects, the present disclosure provides a polypeptide comprising an amino acid sequence having at least 95%, 96%, 97%, 98%, or 99% identity to SEQ ID NO: 3 and comprising a substitution at position L38 (e.g., an L38G substitution) relative to SEQ ID NO: 3. In some aspects, the present disclosure provides a polypeptide comprising an amino acid sequence having one or more sequence alterations (e.g., substitutions, insertions, or deletions, or any combination thereof) at up to 1, 2, 3, 4, 5, 10, 15, 20, 25, 30, or 35 amino acid positions of SEQ ID NO: 3, wherein one of the sequence alterations comprises a substitution at position L38 (e.g., an L38G substitution) relative to SEQ ID NO: 3.

In some aspects, the present disclosure provides a polypeptide comprising an amino acid sequence having at least 95%, 96%, 97%, 98%, or 99% identity to SEQ ID NO: 3 and comprising a substitution at position D145 (e.g., a D145G substitution) relative to SEQ ID NO: 3. In some aspects, the present disclosure provides a polypeptide comprising an amino acid sequence having one or more sequence alterations (e.g., substitutions, insertions, or deletions, or any combination thereof) at up to 1, 2, 3, 4, 5, 10, 15, 20, 25, 30, or 35 amino acid positions of SEQ ID NO: 3, wherein one of the sequence alterations comprises a substitution at position D145 (e.g., a D145G substitution) relative to SEQ ID NO: 3.

In some aspects, the present disclosure provides a polypeptide comprising an amino acid sequence having at least 95%, 96%, 97%, 98%, or 99% identity to SEQ ID NO: 3 and comprising a substitution at position C13 (e.g., a C13G substitution) relative to SEQ ID NO: 3. In some aspects, the present disclosure provides a polypeptide comprising an amino acid sequence having one or more sequence alterations (e.g., substitutions, insertions, or deletions, or any combination thereof) at up to 1, 2, 3, 4, 5, 10, 15, 20, 25, 30, or 35 amino acid positions of SEQ ID NO: 3, wherein one of the sequence alterations comprises a substitution at position C13 (e.g., a C13G substitution) relative to SEQ ID NO: 3.

In some aspects, the present disclosure provides a polypeptide comprising an amino acid sequence having at least 95%, 96%, 97%, 98%, or 99% identity to SEQ ID NO: 3 and comprising a substitution at position T338 (e.g., a T338G substitution) relative to SEQ ID NO: 3. In some aspects, the present disclosure provides a polypeptide comprising an amino acid sequence having one or more sequence alterations (e.g., substitutions, insertions, or deletions, or any combination thereof) at up to 1, 2, 3, 4, 5, 10, 15, 20, 25, 30, or 35 amino acid positions of SEQ ID NO: 3, wherein one of the sequence alterations comprises a substitution at position T338 (e.g., a T338G substitution) relative to SEQ ID NO: 3.

In some aspects, the present disclosure provides a polypeptide comprising an amino acid sequence having at least 95%, 96%, 97%, 98%, or 99% identity to SEQ ID NO: 3 and comprising a substitution at position P14 (e.g., a P14G substitution) relative to SEQ ID NO: 3. In some aspects, the present disclosure provides a polypeptide comprising an amino acid sequence having one or more sequence alterations (e.g., substitutions, insertions, or deletions, or any combination thereof) at up to 1, 2, 3, 4, 5, 10, 15, 20, 25, 30, or 35 amino acid positions of SEQ ID NO: 3, wherein one of the sequence alterations comprises a substitution at position P14 (e.g., a P14G substitution) relative to SEQ ID NO: 3.

In some aspects, the present disclosure provides a polypeptide comprising an amino acid sequence having at least 95%, 96%, 97%, 98%, or 99% identity to SEQ ID NO: 3 and comprising a substitution at position D55 (e.g., a D55G substitution) relative to SEQ ID NO: 3. In some aspects, the present disclosure provides a polypeptide comprising an amino acid sequence having one or more sequence alterations (e.g., substitutions, insertions, or deletions, or any combination thereof) at up to 1, 2, 3, 4, 5, 10, 15, 20, 25, 30, or 35 amino acid positions of SEQ ID NO: 3, wherein one of the sequence alterations comprises a substitution at position D55 (e.g., a D55G substitution) relative to SEQ ID NO: 3.

In some aspects, the present disclosure provides a polypeptide comprising an amino acid sequence having at least 95%, 96%, 97%, 98%, or 99% identity to SEQ ID NO: 3 and comprising a substitution at position K221 (e.g., a K221G substitution) relative to SEQ ID NO: 3. In some aspects, the present disclosure provides a polypeptide comprising an amino acid sequence having one or more sequence alterations (e.g., substitutions, insertions, or deletions, or any combination thereof) at up to 1, 2, 3, 4, 5, 10, 15, 20, 25, 30, or 35 amino acid positions of SEQ ID NO: 3, wherein one of the sequence alterations comprises a substitution at position K221 (e.g., a K221G substitution) relative to SEQ ID NO: 3.

In some aspects, the present disclosure provides a polypeptide comprising an amino acid sequence having at least 95%, 96%, 97%, 98%, or 99% identity to SEQ ID NO: 3 and comprising a substitution at position K35 (e.g., a K35G substitution) relative to SEQ ID NO: 3. In some aspects, the present disclosure provides a polypeptide comprising an amino acid sequence having one or more sequence alterations (e.g., substitutions, insertions, or deletions, or any combination thereof) at up to 1, 2, 3, 4, 5, 10, 15, 20, 25, 30, or 35 amino acid positions of SEQ ID NO: 3, wherein one of the sequence alterations comprises a substitution at position K35 (e.g., a K35G substitution) relative to SEQ ID NO: 3.

In some aspects, the present disclosure provides a polypeptide comprising an amino acid sequence having at least 95%, 96%, 97%, 98%, or 99% identity to SEQ ID NO: 3 and comprising a substitution at position E736 (e.g., an E736G substitution) relative to SEQ ID NO: 3. In some aspects, the present disclosure provides a polypeptide comprising an amino acid sequence having one or more sequence alterations (e.g., substitutions, insertions, or deletions, or any combination thereof) at up to 1, 2, 3, 4, 5, 10, 15, 20, 25, 30, or 35 amino acid positions of SEQ ID NO: 3, wherein one of the sequence alterations comprises a substitution at position E736 (e.g., an E736G substitution) relative to SEQ ID NO: 3.

In some aspects, the variant polypeptide comprises a RuvC domain or a split RuvC domain.

In some aspects, the variant polypeptide comprises one or more catalytic residues (e.g., aspartic acid or glutamic acid). In some aspects, the one or more catalytic residues comprise D328 and E530. In some aspects, the one or more catalytic residues further comprise D684, D646, or D621.

In some aspects, the composition or complex comprising the variant polypeptide further comprises an RNA guide, and the RNA guide comprises a direct repeat sequence and a spacer sequence.

In some aspects, the RNA guide comprises a direct repeat sequence and a spacer sequence.

In some aspects, the direct repeat sequence comprises a nucleotide sequence with at least 95% sequence identity to any one of SEQ ID NOs: 4-13.

In some aspects, the direct repeat sequence comprises the nucleotide sequence of any one of SEQ ID NOs: 4-13.

In some aspects, the spacer sequence comprises between 15 and 35 nucleotides in length.

In some aspects, the target nucleic acid comprises a sequence complementary to a nucleotide sequence in the spacer sequence.

In some aspects, the target nucleic acid is adjacent to a protospacer adjacent motif (PAM) sequence, wherein the PAM sequence comprises a nucleotide sequence set forth as 5′-NTTR-3′, 5′-NTIN-3′, 5′-RTTR-3′, 5′-ATTR-3′, or 5′-RTTG-3′, wherein N is any nucleotide and R is A or G. In some aspects, the PAM sequence comprises a nucleotide sequence set forth as 5′-GTTA-3′, 5′-TTTG-3′, 5′-CTTG-3′, 5′-GTTG-3′, 5′-TTTA-3′, 5′-CTTA-3′, 5′-ATTG-3′, 5′-ATTA-3′, 5′-ACTG-3′, 5′-CATA-3′, 5′-TTGA-3″, or 5′-TATA-3′.

In some aspects, the target nucleic acid is single-stranded DNA or double-stranded DNA.

In some aspects, the variant polypeptide further comprises a peptide tag, a fluorescent protein, a base-editing domain, a DNA methylation domain, a histone residue modification domain, a localization factor, a transcription modification factor, a light-gated control factor, a chemically inducible factor, or a chromatin visualization factor.

In some aspects, a nucleic acid encoding the variant polypeptide is codon-optimized for expression in a cell

In some aspects, the nucleic acid encoding the variant polypeptide is operably linked to a promoter.

In some aspects, the nucleic acid encoding the variant polypeptide is in a vector.

In some aspects, the vector comprises a retroviral vector, a lentiviral vector, a phage vector, an adenoviral vector, an adeno-associated vector, or a herpes simplex vector.

In some aspects, the composition is present in a delivery composition comprising a nanoparticle, a liposome, an exosome, a microvesicle, or a gene-gun.

The invention further provides a cell comprising the variant polypeptide and/or the composition disclosed herein. In some aspects, the cell is a eukaryotic cell or a prokaryotic cell. In some aspects, the cell is a mammalian cell or a plant cell. In some aspects, the cell is a human cell.

The invention further provides a method of preparing the variant polypeptide and/or the composition disclosed herein.

The invention further provides a method of complexing the variant polypeptide with the RNA guide disclosed herein.

The invention further provides a method of complexing the variant binary complex with the target nucleic acid disclosed herein.

The invention further provides a method of delivering the variant polypeptide and/or the composition disclosed herein.

The invention yet further provides a composition comprising a variant polypeptide, or a complex comprising the variant polypeptide and an RNA guide, wherein the variant polypeptide comprises an alteration relative to the parent polypeptide of SEQ ID NO: 3, and wherein the variant polypeptide or the complex exhibits enhanced enzymatic activity, enhanced binding activity, enhanced binding specificity, and/or enhanced stability relative to a parent polypeptide or a complex comprising the parent polypeptide and the RNA guide.

In some aspects, the enhanced enzymatic activity is enhanced nuclease activity.

In some aspects, the variant polypeptide exhibits enhanced binding activity to the RNA guide relative to the parent polypeptide.

In some aspects, the variant polypeptide exhibits enhanced binding specificity to the RNA guide relative to the parent polypeptide.

In some aspects, the variant polypeptide and the RNA guide form a variant binary complex, and the variant binary complex exhibits enhanced binding activity to a target nucleic acid (e.g., on-target binding activity) relative to a parent binary complex.

In some aspects, the variant polypeptide and the RNA guide form a variant binary complex, and the variant binary complex exhibits enhanced binding specificity to a target nucleic acid (e.g., on-target binding specificity) relative to a parent binary complex.

In some aspects, the variant polypeptide and the RNA guide form a variant binary complex, and the variant binary complex exhibits enhanced stability relative to a parent binary complex.

In some aspects, the variant binary complex and a target nucleic acid form a variant ternary complex, and the variant ternary complex exhibits increased stability relative to a parent ternary complex.

In some aspects, the variant polypeptide further exhibits enhanced binary complex formation, enhanced protein-RNA interactions, and/or decreased dissociation from the RNA guide relative to the parent polypeptide.

In some aspects, the variant binary complex further exhibits decreased dissociation from the target nucleic acid, and/or decreased off-target binding to a non-target nucleic acid relative to the parent binary complex.

In some aspects, the enhanced enzymatic activity, enhanced binding activity, enhanced binding specificity, and/or enhanced stability occur over a range of temperatures, e.g., 20° C., to 65° C.

In some aspects, the enhanced enzymatic activity, enhanced binding activity, enhanced binding specificity, and/or enhanced stability occur over a range of incubation times.

In some aspects, the enhanced enzymatic activity, enhanced binding activity, enhanced binding specificity, and/or enhanced stability occur in a buffer having a pH in a range of about 7.3 to about 8.6 (e.g., about 7.5 to about 8.0, about 7.8 to 8.3, or about 8.0 to 8.6).

In some aspects, the enhanced enzymatic activity, enhanced binding activity, enhanced binding specificity, and/or enhanced stability occurs when a Tₘ value of the variant polypeptide, variant binary complex, or variant ternary complex is at least 8° C. greater than the Tₘ value of the parent polypeptide, parent binary complex, or parent ternary complex.

In other aspects, the alteration comprises an amino acid sequence alteration relative to the parent polypeptide having the sequence set forth in SEQ ID NO: 3, wherein the alteration comprises one or more (e.g., one, two, three, four, five, or more) substitutions, insertions, deletions, and/or additions as compared to the parent polypeptide having the sequence set forth in SEQ ID NO:3.

In some aspects, the alteration comprises an amino acid sequence alteration relative to the parent polypeptide sequence set forth in SEQ ID NO: 3, wherein the alteration comprises one or more of the amino acid substitutions listed in Table 2.

In some aspects, the alteration comprises an arginine, lysine, glutamine, asparagine, histidine, alanine, or glycine substitution.

In some aspects, the variant polypeptide comprises a RuvC domain or a split RuvC domain.

In some aspects, the variant polypeptide comprises one or more catalytic residues (e.g., aspartic acid or glutamic acid). In some aspects, the one or more catalytic residues comprise D328 and E530. In some aspects, the one or more catalytic residues further comprise D684, D646, or D621.

In some aspects, the RNA guide comprises a direct repeat sequence and a spacer sequence.

In some aspects, the direct repeat sequence comprises a nucleotide sequence with at least 95% sequence identity to any one of SEQ ID NOs: 4-13.

In some aspects, the direct repeat sequence comprises the nucleotide sequence of any one of SEQ ID NOs: 4-13.

In some aspects, the spacer sequence comprises between 15 and 35 nucleotides in length.

In some aspects, the target nucleic acid comprises a sequence complementary to a nucleotide sequence in the spacer sequence.

In some aspects, the target nucleic acid is adjacent to a PAM sequence, wherein the PAM sequence comprises a nucleotide sequence set forth as 5′-NTTR-3′, 5′-NTTN-3′, 5′-RTTR-3′, 5′-ATTR-3′, or 5′-RTTG-3′, wherein N is any nucleotide and R is A or G. In some aspects, the PAM sequence comprises a nucleotide sequence set forth as 5′-GTTA-3′, 5′-TTTG-3′, 5′-CTTG-3′, 5′-GTTG-3′, 5′-TTTA-3′, 5′-CTTA-3′, 5′-ATTG-3′, 5′-ATTA-3′, 5′-ACTG-3′, 5′-CATA-3′, 5′-TTGA-3′, or 5′-TATA-3″. In some aspects, the target nucleic acid is single-stranded DNA or double-stranded DNA.

In some aspects, the variant polypeptide further comprises a peptide tag, a fluorescent protein, a base-editing domain, a DNA methylation domain, a histone residue modification domain, a localization factor, a transcription modification factor, a light-gated control factor, a chemically inducible factor, or a chromatin visualization factor.

In some aspects, a nucleic acid encoding the variant polypeptide is codon-optimized for expression in a cell

In some aspects, the nucleic acid encoding the variant polypeptide is operably linked to a promoter. In some aspects, the nucleic acid encoding the variant polypeptide is in a vector.

In some aspects, the vector comprises a retroviral vector, a lentiviral vector, a phage vector, an adenoviral vector, an adeno-associated vector, or a herpes simplex vector.

In some aspects, the composition or complex is present in a delivery composition comprising a nanoparticle, a liposome, an exosome, a microvesicle, or a gene-gun.

The invention further provides a cell comprising the variant polypeptide and/or the complex disclosed herein. In some aspects, the cell is a eukaryotic cell or a prokaryotic cell. In some aspects, the cell is a mammalian cell or a plant cell. In some aspects, the cell is a human cell.

The invention further provides a method of preparing the variant polypeptide and/or the complex disclosed herein.

The invention further provides a method of complexing the variant polypeptide with the RNA guide disclosed herein.

The invention further provides a method of complexing the variant binary complex with the target nucleic acid disclosed herein.

The invention further provides a method of delivering the variant polypeptide and/or the complex disclosed herein.

### Definitions

The present invention will be described with respect to particular embodiments and with reference to certain Figures, but the invention is not limited thereto but only by the claims. Terms as set forth hereinafter are generally to be understood in their common sense unless indicated otherwise.

Unless otherwise defined, scientific and technical terms used herein have the meanings that are commonly understood by those of ordinary skill in the art. In the event of any latent ambiguity, definitions provided herein take precedent over any dictionary or extrinsic definition. Unless otherwise required by context, singular terms shall include pluralities and plural terms shall include the singular. The use of “or” means “and/or” unless stated otherwise. The use of the term “including,” as well as other forms, such as “includes” and “included,” is not limiting.

Generally, nomenclature used in connection with cell and tissue culture, molecular biology, immunology, microbiology, genetics, and protein and nucleic acid chemistry and hybridization described herein is well-known and commonly used in the art. The methods and techniques provided herein are generally performed according to conventional methods well known in the art and as described in various general and more specific references that are cited and discussed throughout the present specification unless otherwise indicated. Enzymatic reactions and purification techniques are performed according to manufacturer's specifications, as commonly accomplished in the art or as described herein. The nomenclatures used in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of patients.

That the disclosure may be more readily understood, select terms are defined below.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of +20% or +10%, more preferably +5%, even more preferably +1%, and still more preferably +0.1% from the specified value, as such variations are appropriate to perform the disclosed methods.

As used herein, the term “complex” refers to a grouping of two or more molecules. In some embodiments, the complex comprises a polypeptide and a nucleic acid molecule interacting with (e.g., binding to, coming into contact with, adhering to) one another.

As used herein, the term “binary complex” refers to a grouping of two molecules (e.g., a polypeptide and a nucleic acid molecule). In some embodiments, a binary complex refers to a grouping of a polypeptide and a targeting moiety (e.g., an RNA guide). In some embodiments, a binary complex refers to a ribonucleoprotein (RNP). As used herein, the term “variant binary complex” refers to the grouping of a variant polypeptide and RNA guide. As used herein, the term “parent binary complex” refers to the grouping of a parent polypeptide and RNA guide or a reference polypeptide and RNA guide.

As used herein, the term “ternary complex” refers to a grouping of three molecules (e.g., a polypeptide and two nucleic acid molecules). In some embodiments, a “ternary complex” refers to a grouping of a polypeptide, an RNA molecule, and a DNA molecule. In some embodiments, a ternary complex refers to a grouping of a polypeptide, a targeting moiety (e.g., an RNA guide), and a target nucleic acid (e.g., a target DNA molecule). In some embodiments, a “ternary complex” refers to a grouping of a binary complex (e.g., a ribonucleoprotein) and a third molecule (e.g., a target nucleic acid).

As used herein, the term “domain” refers to a distinct functional and/or structural unit of a polypeptide. In some embodiments, a domain may comprise a conserved amino acid sequence.

As used herein, the terms “parent.” “parent polypeptide.” and “parent sequence” refer to an original polypeptide (e.g., reference or starting polypeptide) to which an alteration is made to produce a variant polypeptide of the present invention.

As used herein, the term “protospacer adjacent motif” or “PAM” refers to a DNA sequence adjacent to a target sequence to which a complex comprising an effector (e.g., a nuclease) and an RNA guide binds. In some embodiments, a PAM is required for enzyme activity. The “target nucleic acid” is a double-stranded molecule: one strand comprises the target sequence adjacent to the PAM and is referred to as the “PAM strand” (e.g., the non-target strand or the non-spacer-complementary strand), and the other complementary strand is referred to as the “non-PAM strand” (e.g., the target strand or the spacer-complementary strand).

As used herein, the term “adjacent” includes instances in which an RNA guide of the complex specifically binds, interacts, or associates with a target sequence that is immediately adjacent to a PAM. In such instances, there are no nucleotides between the target sequence and the PAM. The term “adjacent” also includes instances in which there are a small number (e.g., 1, 2, 3, 4, or 5) of nucleotides between the target sequence, to which the targeting moiety binds, and the PAM.

As used herein, the terms “reference composition,” “reference molecule,” “reference sequence.” and “reference” refer to a control, such as a negative control or a parent (e.g., a parent sequence, a parent protein, or a wild-type protein). For example, a reference molecule refers to a polypeptide to which a variant polypeptide is compared. Likewise, a reference RNA guide refers to a targeting moiety to which a modified RNA guide is compared. The variant or modified molecule may be compared to the reference molecule on the basis of sequence (e.g., the variant or modified molecule may have X % sequence identity or homology with the reference molecule), thermostability, or activity (e.g., the variant or modified molecule may have X % of the activity of the reference molecule). For example, a variant or modified molecule may be characterized as having no more than 10% of an activity of the reference polypeptide or may be characterized as having at least 10% greater of an activity of the reference polypeptide. Examples of reference polypeptides include naturally occurring unmodified polypeptides, e.g., naturally occurring polypeptides from archaca or bacterial species. In certain embodiments, the reference polypeptide is a naturally occurring polypeptide having the closest sequence identity or homology with the variant polypeptide to which it is being compared. In certain embodiments, the reference polypeptide is a parental molecule having a naturally occurring or known sequence on which a mutation has been made to arrive at the variant polypeptide.

As used herein, the terms “RNA guide” or “RNA guide sequence” refer to any RNA molecule that facilitates the targeting of a polypeptide described herein to a target nucleic acid. For example, an RNA guide can be a molecule that recognizes (e.g., binds to) a target nucleic acid. An RNA guide may be designed to be complementary to a target strand (e.g., the non-PAM strand) of a target nucleic acid sequence. An RNA guide comprises a DNA targeting sequence and a direct repeat (DR) sequence. The terms CRISPR RNA (crRNA), pre-crRNA, mature crRNA, and gRNA are also used herein to refer to an RNA guide. As used herein, the term “pre-crRNA” refers to an unprocessed RNA molecule comprising a DR-spacer-DR sequence. As used herein, the term “mature crRNA” refers to a processed form of a pre-crRNA: a mature crRNA may comprise a DR-spacer sequence, wherein the DR is a truncated form of the DR of a pre-crRNA and/or the spacer is a truncated form of the spacer of a pre-crRNA. As used herein, the term “substantially identical” refers to a sequence, polynucleotide, or polypeptide, that has a certain degree of identity to a reference sequence.

As used herein, the term “substantially identical” refers to a sequence, polynucleotide, or polypeptide, that has a certain degree of identity to a reference sequence.

As used herein, the terms “target nucleic acid,” “target sequence,” and “target substrate” refer to a nucleic acid to which an RNA guide specifically binds. In some embodiments, the DNA targeting sequence of an RNA guide binds to a target nucleic acid.

As used herein, the terms “variant polypeptide”, “variant effector polypeptide,” and “variant CRISPR nuclease polypeptide” refer to a polypeptide comprising an alteration, e.g., but not limited to, a substitution, insertion, deletion, addition and/or fusion, at one or more residue positions, compared to a parent polypeptide. As used herein, the terms “variant polypeptide”, variant effector polypeptide,” and “variant CRISPR nuclease polypeptide” refer to a polypeptide comprising an alteration as compared to the polypeptide of SEQ ID NO: 3.

### BRIEF DESCRIPTION OF THE DRAWINGS

FIG. 1A shows indel activity (% raw indel) of CRISPR nuclease variants comprising two or three arginine substitutions compared to a wild-type CRISPR nuclease (WT: SEQ ID NO: 3) across an EMX1 target sequence (SEQ ID NO: 16), an AAVSI target sequence (SEQ ID NO: 20), and a VEGFA target sequence (SEQ ID NO: 18). Data shown is an average of two bioreplicates of two technical replicates each.

FIG. 1B shows indel activity (% raw indel) of CRISPR nuclease variants comprising two, three, or four arginine substitutions compared to a wild-type CRISPR nuclease (WT: SEQ ID NO: 3) across an EMX1 target sequence (SEQ ID NO: 16), an AAVSI target sequence (SEQ ID NO: 20), and a VEGFA target sequence (SEQ ID NO: 18). Data shown is an average of two bioreplicates of two technical replicates each.

FIG. 1C shows indel activity (% raw indel) of CRISPR nuclease variants comprising two, three, or four arginine substitutions compared to a wild-type CRISPR nuclease (WT: SEQ ID NO: 3) across an EMX1 target sequence (SEQ ID NO: 16), an AAVSI target sequence (SEQ ID NO: 20), and a VEGFA target sequence (SEQ ID NO: 18). Data shown is an average of two bioreplicates of two technical replicates each.

FIG. 2 shows indel activity (% raw indel) of CRISPR nuclease variants comprising single glycine substitutions compared to a wild-type CRISPR nuclease (WT: SEQ ID NO: 3) across an EMX1 target sequence (SEQ ID NO: 16), an AAVSI target sequence (SEQ ID NO: 20), and a VEGFA target sequence (SEQ ID NO: 18). The dotted lines depict the average indel activity by the parent polypeptide of SEQ ID NO: 3 at each of the three targets. Data shown is an average of two bioreplicates of two technical replicates each.

### DETAILED DESCRIPTION

In some aspects, the present invention provides novel variants of the effector (e.g., the CRISPR nuclease) of SEQ ID NO: 3, compositions comprising the variants, and methods of preparation and use thereof. In other aspects, the present invention further provides complexes comprising a variant of the effector (e.g., the CRISPR nuclease) of SEQ ID NO: 3 and compositions, methods of preparation and use thereof. In some aspects, a composition comprising a complex having one or more characteristics is described herein. In some aspects, a method of delivering a composition comprising the complex is described.

### Compositions

In some embodiments, a composition of the invention includes a variant polypeptide that exhibits enhanced enzymatic activity, enhanced binding activity, enhanced binding specificity, and/or enhanced stability relative to a parent polypeptide. In some embodiments, a composition of the invention includes a complex comprising a variant polypeptide that exhibits enhanced enzymatic activity, enhanced binding activity, enhanced binding specificity, and/or enhanced stability relative to a parent complex.

In some embodiments, a composition of the invention includes a variant polypeptide and an RNA guide. In some embodiments, a composition of the invention includes a variant binary complex comprising a variant polypeptide and an RNA guide.

In some aspects of the composition, the variant polypeptide has increased complex formation (e.g., increased binary complex formation) with the RNA guide as compared to a parent polypeptide. In some aspects of the composition, the variant polypeptide and the RNA guide have a greater binding affinity, as compared to a parent polypeptide and the RNA guide. In some aspects of the composition, the variant polypeptide and the RNA guide have stronger protein-RNA interactions (e.g., ionic interactions), as compared to a parent polypeptide and the RNA guide. In some aspects of the composition, the variant binary complex is more stable than a parent binary complex.

In some embodiments, a composition of the invention includes a variant polypeptide, an RNA guide, and a target nucleic acid. In some embodiments, a composition of the invention includes a variant temary complex comprising a variant polypeptide, an RNA guide, and a target nucleic acid.

In some aspects of the composition, the variant polypeptide has increased complex formation (e.g., increased ternary complex formation) with the RNA guide and target nucleic acid as compared to a parent polypeptide. In some aspects of the composition, the variant polypeptide and the RNA guide (e.g., the variant binary complex) have a greater binding affinity to a target nucleic acid, as compared to a parent polypeptide and the RNA guide (e.g., a parent binary complex). In some aspects of the composition, the variant ternary complex is more stable than a parent temary complex.

In some embodiments, the composition of the present invention includes a variant polypeptide described herein.

### Variant Polypeptides

In one embodiment, the variant polypeptide is an isolated or purified polypeptide.

In some embodiments, the variant polypeptide (e.g., variant CRISPR nuclease polypeptide) of the present invention is a variant of a parent polypeptide (e.g., a parent CRISPR nuclease), wherein the parent is encoded by a polynucleotide that comprises a nucleotide sequence such as SEQ ID NO: 1, SEQ ID NO: 2, SEQ ID NO: 21, SEQ ID NO: 22, SEQ ID NO: 23, SEQ ID NO: 24, SEQ ID NO: 25, SEQ ID NO: 26, or SEQ ID NO: 27 or comprises an amino acid sequence such as SEQ ID NO: 3. See Table 1.

TABLE 1
Sequences corresponding to SEQ ID NOs: 1-3 and 21-27.

SEQ ID NO: 1
ATGATCAAGTCTATTCAGCTGAAGGTCAAGGGGGAGTGTCCGATCACGAAGGATGTAATCAACGAATAC
AAAGAATACTATAACAATTGCAGTGATTGGATTAAAAACAATCTGACGTCCATTACCATCGGGGAGATG
GCAAAATTTCTGCAATCGTTGAGCGATAAAGAAGTGGCCTATATCTCAATGGGCCTGTCCGATGAGTGG
AAAGACAAACCGTTATATCATCTGTTTACCAAAAAATATCACACCAAAAATGCGGATAACTTATTATAC
TACTACATTAAAGAAAAAAATCTGGACGGCTACAAAGGCAATACGCTTAATATCTCCAATACATCTTTC
CGCCAGTTCGGTTATTTCAAACTCGTGGTGAGCAACTACCGCACTAAAATCCGTACGCTGAATTGTAAA
ATCAAGCGTAAGAAAATCGATGCCGATTCCACGTCTGAGGATATCGAAATGCAGGTGATGTACGAAATT
ATTAAATACAGTTTAAATAAAAAGTCTGATTGGGATAACTTCATTAGCTATATCGAAAACGTTGAAAAT
CCTAATATTGACAACATCAACCGCTACAAACTGCTGCGCGAATGCTTTTGCGAAAACGAAAACATGATT
AAGAACAAACTTGAATTGCTGTCTGTGGAACAATTGAAAAAATTTGGCGGTTGCATCATGAAACCTCAC
ATTAATAGCATGACCATTAACATTCAAGATTTTAAAATTGAGGAAAAAGAAAACTCTCTGGGGTTCATC
CTCCACCTCCCACTGAACAAAAAACAGTATCAAATTGAACTCCTGGGCAATCGTCAGATTAAAAAAGGC
ACCAAAGAAATTCACGAAACGTTAGTTGACATTACTAACACCCATGGCGAAAACATTGTGTTTACTATT
AAAAATGATAATCTGTATATCGTGTTCTCTTATGAGTCCGAATTTGAAAAAGAAGAAGTTAACTTCGCT
AAAACGGTTGGCCTGGACGTAAACTTCAAACATGCCTTCTTTGTGACCTCTGAGAAAGATAACTGTCAT
CTCGACGGTTATATTAATCTCTACAAATACTTATTGGAGCACGACGAGTTTACTAACCTTCTCACCGAA
GATGAACGTAAAGATTATGAGGAGCTGAGTAAAGTCGTTACTTTCTGCCCGTTTGAAAATCAGTTACTG
TTTGCGCGTTACAACAAGATGAGCAAATTCTGCAAGAAAGAACAGGTCCTGAGCAAACTTCTCTATGCG
CTGCAGAAAAAACTCAAAGACGAGAACCGCACGAAAGAATATATTTATGTCTCGTGCGTGAACAAATTA
CGTGCCAAATATGTGTCATATTTTATTCTGAAAGAAAAGTACTACGAGAAGCAGAAAGAATATGACATC
GAAATGGGCTTTGTGGACGACTCAACGGAAAGCAAAGAATCAATGGATAAACGCCGTACTGAATATCCG
TTTCGCAACACGCCGGTAGCCAACGAACTGTTGTCCAAACTGAATAACGTACAGCAGGACATCAACGGG
TGCCTGAAGAACATCATCAACTACATTTATAAAATTTTCGAGCAGAACGGTTATAAAGTTGTCGCCCTC
GAAAACCTGGAAAATTCTAATTTTGAAAAAAAACAGGTGTTGCCGACGATTAAAAGTCTGCTGAAATAT
CACAAACTGGAGAACCAGAACGTGAATGATATCAAGGCCTCTGACAAAGTTAAAGAATATATTGAAAAC
GGTTATTATGAACTCATGACCAACGAGAATAACGAAATCGTTGATGCAAAATATACAGAAAAGGGCGCA
ATGAAGGTGAAGAACGCCAATTTTTTTAACCTCATGATGAAAAGTTTGCATTTTGCCAGTGTGAAAGAT
GAGTTTGTGCTGCTGTCCAATAATGGCAAGACGCAGATTGCATTAGTGCCATCCGAGTTTACATCTCAG
ATGGACAGCACCGATCACTGTCTGTACATGAAGAAGAACGACAAAGGTAAACTGGTGAAAGCGGATAAA
AAGGAAGTTCGTACAAAACAGGAACGTCACATCAACGGCCTCAACGCCGATTTCAACGCAGCGAATAAT
ATTAAATATATCGTGGAAAATGAAGTGTGGCGTGGTATTTTTTGCACTCGCCCGAAGAAAACAGAATAT
AACGTACCCAGTCTGGATACCACGAAAAAAGGTCCGTCTGCGATTCTCAACATGCTGAAGAAAATTGAA
GCCATCAAGGTCCTGGAAACGGAAAAA
SEQ ID NO: 2
ATGATCAAGAGCATCCAGCTGAAGGTGAAGGGCGAGTGCCCCATCACCAAGGACGTGATCAACGAGTAC
AAGGAGTACTACAACAACTGTTCTGATTGGATCAAGAACAATCTGACCAGCATCACAATCGGCGAGATG
GCCAAGTTTCTGCAGAGCCTGTCCGACAAGGAGGTGGCCTACATCTCTATGGGCCTGAGCGACGAGTGG
AAGGATAAGCCTCTGTATCACCTGTTCACCAAGAAGTACCACACAAAGAATGCCGACAACCTGCTGTAC
TATTACATCAAGGAGAAGAACCTGGATGGCTACAAGGGCAATACCCTGAACATCTCCAATACATCTTTC
AGGCAGTTTGGCTATTTCAAGCTGGTGGTGTCCAATTACAGGACCAAGATCCGCACACTGAACTGCAAG
ATCAAGCGCAAGAAGATCGACGCCGATTCTACCAGCGAGGACATCGAGATGCAGGTCATGTATGAGATC
ATCAAGTACTCCCTGAACAAGAAGTCTGATTGGGATAATTTCATCTCTTATATCGAGAACGTGGAGAAC
CCCAATATCGATAACATCAATCGGTACAAGCTGCTGAGAGAGTGCTTTTGTGAGAACGAGAATATGATC
AAGAACAAGCTGGAGCTGCTGAGCGTGGAGCAGCTGAAGAAGTTCGGCGGCTGTATCATGAAGCCTCAC
ATCAACAGCATGACCATCAATATCCAGGACTTTAAGATCGAGGAGAAGGAGAATTCCCTGGGCTTCATC
CTGCACCTGCCACTGAACAAGAAGCAGTACCAGATCGAGCTGCTGGGCAATCGGCAGATCAAGAAGGGC
ACCAAGGAGATCCACGAGACACTGGTGGACATCACCAACACACACGGCGAGAACATCGTGTTTACAATC
AAGAACGATAATCTGTACATCGTGTTTAGCTACGAGTCCGAGTTCGAGAAGGAGGAAGTGAATTTTGCC
AAGACCGTGGGCCTGGACGTGAACTTCAAGCACGCCTTCTTTGTGACATCCGAGAAGGACAATTGCCAC
CTGGATGGCTATATCAACCTGTATAAGTACCTGCTGGAGCACGATGAGTTCACCAACCTGCTGACAGAG
GACGAGCGGAAGGATTACGAGGAGCTGTCTAAGGTGGTGACCTTTTGCCCTTTCGAGAATCAGCTGCTG
TTTGCCAGATATAACAAGATGAGCAAGTTCTGTAAGAAGGAGCAGGTGCTGTCCAAGCTGCTGTACGCC
CTGCAGAAGAAGCTGAAGGACGAGAACAGGACAAAGGAGTATATCTACGTGTCTTGCGTGAATAAGCTG
CGCGCCAAGTATGTGAGCTACTTTATCCTGAAGGAGAAGTATTACGAGAAGCAGAAGGAGTATGACATC
GAGATGGGCTTCGTGGACGATTCTACCGAGAGCAAGGAGTCCATGGATAAGCGGAGAACCGAGTACCCC
TTTAGGAACACACCTGTGGCCAATGAGCTGCTGAGCAAGCTGAACAATGTGCAGCAGGATATCAACGGC
TGTCTGAAGAACATCATCAATTACATCTACAAGATCTTCGAGCAGAACGGCTACAAGGTGGTGGCCCTG
GAGAACCTGGAGAACTCCAATTTCGAGAAGAAGCAGGTGCTGCCAACAATCAAGTCTCTGCTGAAGTAT
CACAAGCTGGAGAACCAGAATGTGAACGACATCAAGGCCAGCGATAAGGTGAAGGAGTACATCGAGAAT
GGCTATTACGAGCTGATGACCAACGAGAACAATGAGATCGTGGACGCCAAGTATACAGAGAAGGGCGCC
ATGAAGGTGAAGAATGCCAACTTCTTTAACCTGATGATGAAGAGCCTGCACTTTGCCTCCGTGAAGGAT
GAGTTCGTGCTGCTGTCCAACAATGGCAAGACCCAGATCGCCCTGGTGCCATCCGAGTTCACCTCTCAG
ATGGACAGCACAGATCACTGCCTGTACATGAAGAAGAATGACAAGGGCAAGCTGGTGAAGGCCGATAAG
AAGGAGGTGAGGACAAAGCAGGAGCGCCACATCAACGGCCTGAATGCCGACTTTAACGCCGCCAACAAT
ATCAAGTATATCGTGGAGAATGAAGTGTGGCGGGGCATCTTCTGTACCAGACCAAAGAAGACAGAGTAC
AACGTGCCCTCCCTGGATACCACAAAGAAGGGCCCCTCTGCCATCCTGAATATGCTGAAGAAGATCGAG
GCCATCAAGGTGCTGGAGACCGAGAAG
SEQ ID NO: 3
MIKSIQLKVKGECPITKDVINEYKEYYNNCSDWIKNNLTSITIGEMAKFLQSLSDKEVAYISMGLSDEW
KDKPLYHLFTKKYHTKNADNLLYYYIKEKNLDGYKGNTLNISNTSFRQFGYFKLVVSNYRTKIRTLNCK
IKRKKIDADSTSEDIEMQVMYEIIKYSLNKKSDWDNFISYIENVENPNIDNINRYKLLRECFCENENMI
KNKLELLSVEQLKKFGGCIMKPHINSMTINIQDFKIEEKENSLGFILHLPLNKKQYQIELLGNRQIKKG
TKEIHETLVDITNTHGENIVFTIKNDNLYIVFSYESEFEKEEVNFAKTVGLDVNFKHAFFVTSEKDNCH
LDGYINLYKYLLEHDEFTNLLTEDERKDYEELSKVVTFCPFENQLLFARYNKMSKFCKKEQVLSKLLYA
LQKKLKDENRTKEYIYVSCVNKLRAKYVSYFILKEKYYEKQKEYDIEMGFVDDSTESKESMDKRRTEYP
FRNTPVANELLSKLNNVQQDINGCLKNIINYIYKIFEQNGYKVVALENLENSNFEKKQVLPTIKSLLKY
HKLENQNVNDIKASDKVKEYIENGYYELMTNENNEIVDAKYTEKGAMKVKNANFFNLMMKSLHFASVKD
EFVLLSNNGKTQIALVPSEFTSQMDSTDHCLYMKKNDKGKLVKADKKEVRTKQERHINGLNADFNAANN
IKYIVENEVWRGIFCTRPKKTEYNVPSLDTTKKGPSAILNMLKKIEAIKVLETEK
SEQ ID NO: 21
ATGATTAAATCCATCCAGCTTAAAGTGAAAGGCGAATGCCCCATCACCAAAGATGTTATTAACGAGTAC
AAGGAATACTATAATAACTGCAGCGATTGGATAAAAAACAATCTTACCTCGATCACTATTGGCGAGATG
GCCAAGTTCCTGCAGAGCCTGTCTGATAAGGAGGTTGCTTACATTTCCATGGGCCTGAGCGACGAGTGG
AAAGACAAGCCCTTGTACCACCTGTTCACTAAGAAATACCACACTAAGAATGCTGACAATCTGCTCTAC
TACTACATCAAGGAGAAAAACTTGGATGGCTACAAGGGGAACACGCTTAACATTTCTAACACCTCATTT
CGTCAGTTCGGGTATTTCAAGCTCGTGGTGTCCAACTATCGCACTAAGATTCGGACACTCAACTGCAAG
ATTAAACGAAAGAAGATCGACGCAGATTCTACGAGCGAGGATATTGAAATGCAGGTCATGTATGAAATC
ATCAAGTACTCTCTGAATAAGAAGTCTGACTGGGACAATTTTATCAGCTACATCGAAAACGTAGAGAAC
CCTAACATAGACAATATCAACAGGTACAAACTGCTTAGGGAATGCTTCTGTGAAAACGAGAATATGATA
AAAAACAAGCTGGAGCTGCTGAGCGTGGAGCAATTAAAGAAGTTTGGAGGGTGTATCATGAAACCCCAC
ATTAACTCGATGACAATTAATATTCAAGACTTCAAGATAGAGGAGAAAGAAAACAGCTTGGGCTTTATT
CTCCATCTCCCTCTGAATAAAAAACAGTACCAGATCGAGCTATTGGGAAATAGACAGATTAAGAAAGGG
ACCAAGGAAATTCACGAAACTCTCGTCGATATCACAAACACCCATGGAGAGAACATCGTGTTCACAATC
AAGAACGATAATCTGTACATAGTGTTTAGTTATGAGAGCGAGTTCGAGAAGGAAGAGGTCAACTTTGCC
AAGACTGTTGGGCTTGACGTCAATTTCAAACACGCGTTCTTCGTGACAAGCGAAAAGGACAACTGCCAT
CTCGATGGCTACATTAACCTATATAAGTATTTGCTCGAACACGACGAGTTTACAAACCTGCTGACAGAA
GATGAGAGAAAGGACTACGAAGAACTCAGTAAGGTCGTTACGTTCTGTCCATTTGAAAATCAGCTGCTA
TTCGCCCGGTACAATAAAATGTCCAAATTCTGTAAGAAGGAACAGGTATTGTCTAAACTGCTGTATGCC
CTGCAGAAAAAGTTGAAAGATGAGAATCGGACCAAAGAGTATATCTATGTCTCATGCGTGAACAAGCTA
AGAGCTAAGTATGTTTCCTATTTCATACTGAAGGAGAAGTACTATGAGAAGCAAAAGGAGTACGACATT
GAGATGGGCTTCGTCGATGACTCAACCGAATCTAAAGAATCCATGGACAAAAGGCGCACAGAGTATCCA
TTTAGAAATACACCGGTGGCTAACGAACTCCTGAGTAAACTCAACAATGTTCAACAAGATATCAACGGT
TGTCTGAAGAATATAATTAATTATATCTATAAGATTTTTGAACAGAATGGCTACAAGGTGGTCGCACTG
GAAAACTTAGAGAATTCCAACTTTGAGAAAAAGCAGGTGCTTCCTACAATCAAATCACTTCTGAAGTAC
CACAAACTTGAGAATCAGAACGTAAATGATATCAAAGCCAGTGATAAAGTCAAAGAATACATCGAGAAT
GGTTATTATGAACTGATGACTAATGAAAATAATGAAATAGTGGACGCAAAATATACGGAAAAGGGTGCC
ATGAAGGTAAAGAACGCAAACTTTTTTAATTTGATGATGAAGTCACTGCACTTTGCTTCTGTGAAGGAT
GAGTTTGTCCTGCTGAGCAACAACGGAAAGACACAGATTGCGCTAGTGCCTTCAGAGTTCACTAGTCAG
ATGGACAGTACCGACCATTGCCTCTACATGAAAAAAAATGACAAAGGGAAACTCGTGAAGGCTGACAAG
AAAGAGGTGCGGACCAAGCAAGAGCGCCATATCAATGGATTAAACGCCGATTTTAACGCGGCAAATAAC
ATCAAATACATCGTTGAAAATGAGGTGTGGAGGGGTATCTTCTGTACCCGACCAAAGAAGACTGAGTAC
AACGTACCATCCTTAGACACCACCAAAAAAGGACCCTCCGCCATTCTGAATATGTTAAAGAAAATCGAG
GCCATAAAAGTGTTGGAGACCGAGAAG
SEQ ID NO: 22
ATGATCAAGAGCATTCAGCTGAAGGTGAAGGGCGAGTGCCCCATCACCAAGGACGTGATCAACGAGTAC
AAGGAGTACTACAACAACTGCAGCGACTGGATTAAAAACAATCTCACAAGCATCACCATCGGCGAGATG
GCCAAGTTCCTGCAGAGCCTGAGCGACAAAGAGGTGGCCTACATCAGCATGGGCCTGAGCGACGAGTGG
AAGGACAAGCCCCTGTACCACCTGTTCACCAAGAAGTACCACACCAAGAACGCCGACAACCTGCTGTAC
TACTACATCAAGGAGAAGAATCTGGATGGCTACAAGGGCAATACCCTGAACATCAGCAACACCAGCTTC
AGACAGTTCGGCTACTTCAAGCTGGTGGTGAGCAACTACAGAACCAAGATCAGAACCCTGAACTGCAAG
ATCAAGAGAAAGAAGATCGACGCCGACAGCACAAGCGAGGACATAGAGATGCAAGTTATGTACGAGATC
ATCAAGTACAGCCTTAATAAAAAGAGCGACTGGGACAACTTCATCAGCTACATAGAAAACGTGGAGAAC
CCCAACATCGACAACATCAACAGATACAAGCTGCTGAGAGAGTGCTTCTGCGAGAACGAGAACATGATC
AAAAACAAACTCGAACTGCTGAGTGTAGAACAGCTGAAGAAGTTCGGCGGCTGCATCATGAAGCCCCAC
ATCAACAGCATGACCATCAACATCCAAGACTTCAAGATCGAGGAGAAGGAGAACAGCCTGGGCTTCATC
CTGCACCTGCCCTTAAACAAGAAGCAGTATCAGATCGAGCTGCTGGGCAACAGACAGATCAAGAAGGGC
ACCAAGGAGATCCACGAGACCCTGGTGGACATCACCAACACCCACGGCGAGAATATCGTTTTCACTATC
AAGAACGACAACCTGTACATCGTGTTTAGCTATGAGAGCGAGTTCGAGAAGGAAGAGGTGAACTTCGCC
AAGACCGTGGGCCTGGACGTGAACTTCAAGCACGCCTTCTTCGTGACAAGCGAGAAGGACAACTGCCAC
CTGGACGGCTACATCAATCTGTACAAGTACCTGCTGGAGCACGACGAGTTCACCAACCTGCTGACCGAG
GACGAGAGAAAGGACTACGAGGAGCTGAGCAAGGTGGTGACCTTCTGCCCCTTCGAGAATCAGCTGCTG
TTCGCTAGATACAACAAGATGAGCAAGTTCTGCAAGAAGGAGCAAGTGCTGAGCAAACTGCTGTACGCC
CTGCAGAAGAAGCTGAAGGACGAGAACAGAACCAAGGAGTATATCTACGTGAGCTGCGTGAACAAGCTG
AGAGCCAAGTACGTGAGCTACTTCATCCTGAAGGAGAAGTACTACGAGAAGCAGAAGGAGTACGACATC
GAGATGGGTTTTGTGGACGACAGCACCGAGAGCAAGGAGAGCATGGACAAGAGAAGAACCGAGTACCCC
TTCAGAAACACCCCCGTGGCCAACGAATTACTGTCTAAACTGAATAACGTGCAGCAAGACATCAACGGC
TGCCTGAAGAACATAATCAACTACATCTACAAGATCTTCGAGCAGAACGGCTACAAGGTGGTAGCCCTG
GAGAACCTGGAGAACAGCAACTTCGAGAAGAAGCAAGTGCTGCCCACCATCAAGAGCCTGCTGAAGTAC
CACAAGCTGGAGAATCAGAACGTGAACGACATCAAGGCTAGCGACAAGGTGAAGGAGTACATCGAGAAC
GGATACTACGAGCTGATGACCAACGAGAACAACGAGATCGTGGACGCCAAGTACACCGAGAAGGGCGCC
ATGAAGGTGAAGAACGCCAACTTCTTCAACCTGATGATGAAGAGCCTGCACTTCGCTAGCGTGAAGGAC
GAGTTCGTGCTGCTGTCGAACAACGGCAAGACACAGATCGCCCTGGTGCCTAGCGAGTTCACATCTCAG
ATGGACAGCACCGACCACTGCCTGTACATGAAGAAGAACGACAAGGGCAAGCTGGTGAAGGCCGACAAG
AAAGAGGTGAGAACCAAGCAAGAGAGACACATCAACGGCCTGAACGCCGACTTCAACGCCGCCAACAAC
ATCAAGTACATCGTGGAGAACGAGGTGTGGAGAGGCATCTTCTGCACAAGACCCAAGAAGACCGAGTAC
AACGTGCCTAGCCTGGACACCACCAAGAAGGGCCCTAGCGCCATCCTGAACATGCTGAAGAAGATCGAG
GCCATCAAGGTGCTGGAGACCGAGAAG
SEQ ID NO: 23
ATGATCAAGAGCATCCAGCTGAAGGTGAAGGGCGAGTGCCCCATCACCAAGGACGTGATCAACGAGTAC
AAGGAGTACTACAACAACTGCAGCGACTGGATCAAGAACAACCTGACCAGCATCACCATCGGCGAGATG
GCCAAGTTCCTGCAGAGCCTGAGCGACAAGGAGGTGGCCTACATCAGCATGGGCCTGAGCGACGAGTGG
AAGGACAAGCCCCTGTACCACCTGTTCACCAAGAAGTACCACACCAAGAACGCCGACAACCTGCTGTAC
TACTACATCAAGGAGAAGAACCTGGACGGCTACAAGGGCAACACCCTGAACATCAGCAACACCAGCTTC
CGGCAGTTCGGCTACTTCAAGCTGGTGGTGAGCAACTACCGGACCAAGATCCGGACCCTGAACTGCAAG
ATCAAGCGGAAGAAGATCGACGCCGACAGCACCAGCGAGGACATCGAGATGCAGGTGATGTACGAGATC
ATCAAGTACAGCCTGAACAAGAAGAGCGACTGGGACAACTTCATCAGCTACATCGAGAACGTGGAGAAC
CCCAACATCGACAACATCAACCGGTACAAGCTGCTGCGGGAGTGCTTCTGCGAGAACGAGAACATGATC
AAGAACAAGCTGGAGCTGCTGAGCGTGGAGCAGCTGAAGAAGTTCGGCGGCTGCATCATGAAGCCCCAC
ATCAACAGCATGACCATCAACATCCAGGACTTCAAGATCGAGGAGAAGGAGAACAGCCTGGGCTTCATC
CTGCACCTGCCCCTGAACAAGAAGCAGTACCAGATCGAGCTGCTGGGCAACCGGCAGATCAAGAAGGGC
ACCAAGGAGATCCACGAGACCCTGGTGGACATCACCAACACCCACGGCGAGAACATCGTGTTCACCATC
AAGAACGACAACCTGTACATCGTGTTCAGCTACGAGAGCGAGTTCGAGAAGGAGGAGGTGAACTTCGCC
AAGACCGTGGGCCTGGACGTGAACTTCAAGCACGCCTTCTTCGTGACCAGCGAGAAGGACAACTGCCAC
CTGGACGGCTACATCAACCTGTACAAGTACCTGCTGGAGCACGACGAGTTCACCAACCTGCTGACCGAG
GACGAGCGGAAGGACTACGAGGAGCTGAGCAAGGTGGTGACCTTCTGCCCCTTCGAGAACCAGCTGCTG
TTCGCCCGGTACAACAAGATGAGCAAGTTCTGCAAGAAGGAGCAGGTGCTGAGCAAGCTGCTGTACGCC
CTGCAGAAGAAGCTGAAGGACGAGAACCGGACCAAGGAGTACATCTACGTGAGCTGCGTGAACAAGCTG
CGGGCCAAGTACGTGAGCTACTTCATCCTGAAGGAGAAGTACTACGAGAAGCAGAAGGAGTACGACATC
GAGATGGGCTTCGTGGACGACAGCACCGAGAGCAAGGAGAGCATGGACAAGCGGCGGACCGAGTACCCC
TTCCGGAACACCCCCGTGGCCAACGAGCTGCTGAGCAAGCTGAACAACGTGCAGCAGGACATCAACGGC
TGCCTGAAGAACATCATCAACTACATCTACAAGATCTTCGAGCAGAACGGCTACAAGGTGGTGGCCCTG
GAGAACCTGGAGAACAGCAACTTCGAGAAGAAGCAGGTGCTGCCCACCATCAAGAGCCTGCTGAAGTAC
CACAAGCTGGAGAACCAGAACGTGAACGACATCAAGGCCAGCGACAAGGTGAAGGAGTACATCGAGAAC
GGCTACTACGAGCTGATGACCAACGAGAACAACGAGATCGTGGACGCCAAGTACACCGAGAAGGGCGCC
ATGAAGGTGAAGAACGCCAACTTCTTCAACCTGATGATGAAGAGCCTGCACTTCGCCAGCGTGAAGGAC
GAGTTCGTGCTGCTGAGCAACAACGGCAAGACCCAGATCGCCCTGGTGCCCAGCGAGTTCACCAGCCAG
ATGGACAGCACCGACCACTGCCTGTACATGAAGAAGAACGACAAGGGCAAGCTGGTGAAGGCCGACAAG
AAGGAGGTGCGGACCAAGCAGGAGCGGCACATCAACGGCCTGAACGCCGACTTCAACGCCGCCAACAAC
ATCAAGTACATCGTGGAGAACGAGGTGTGGCGGGGCATCTTCTGCACCCGGCCCAAGAAGACCGAGTAC
AACGTGCCCAGCCTGGACACCACCAAGAAGGGCCCCAGCGCCATCCTGAACATGCTGAAGAAGATCGAG
GCCATCAAGGTGCTGGAGACCGAGAAG
SEQ ID NO: 24
ATGATTAAAAGTATACAGCTTAAGGTCAAGGGCGAGTGCCCGATTACCAAAGATGTAATTAACGAGTAC
AAGGAGTATTACAATAACTGTTCTGACTGGATCAAGAACAATCTGACTTCAATTACGATCGGTGAAATG
GCCAAATTTCTCCAGTCCTTGAGTGATAAGGAGGTCGCATATATATCCATGGGACTCAGTGACGAGTGG
AAGGACAAACCCCTGTACCATCTTTTTACTAAAAAATACCATACGAAAAACGCAGACAACCTGCTGTAC
TACTATATAAAAGAAAAAAACCTGGACGGTTACAAGGGCAACACCCTCAATATTAGCAACACTAGTTTC
CGACAATTCGGGTACTTCAAGCTGGTCGTGAGCAACTATCGGACCAAAATCAGGACTTTGAATTGTAAG
ATAAAGAGAAAGAAGATAGACGCAGATTCTACTAGTGAGGATATCGAGATGCAGGTAATGTACGAGATC
ATTAAGTATTCACTGAACAAGAAGAGCGATTGGGATAACTTCATATCATACATCGAAAACGTTGAAAAT
CCAAACATCGACAATATTAATAGATACAAACTGTTGAGAGAATGCTTCTGCGAAAACGAAAATATGATA
AAAAACAAACTCGAATTGTTGTCAGTTGAACAGCTGAAGAAATTCGGAGGGTGCATAATGAAGCCTCAC
ATAAACTCAATGACAATAAACATCCAAGACTTCAAGATAGAAGAGAAGGAGAACAGTTTGGGTTTTATT
CTTCACCTGCCTTTGAACAAGAAGCAATACCAAATCGAGCTGCTCGGAAATAGGCAGATAAAAAAGGGG
ACAAAAGAAATACACGAAACCCTTGTTGACATTACGAACACACACGGGGAAAATATCGTGTTCACAATT
AAAAATGACAACCTTTACATTGTATTTTCTTATGAATCAGAGTTCGAAAAGGAAGAAGTGAACTTTGCC
AAGACCGTTGGTTTGGACGTCAACTTTAAACACGCCTTCTTCGTTACATCAGAAAAGGATAACTGTCAT
CTTGATGGATACATAAACCTCTACAAGTATCTTCTTGAACACGACGAATTCACAAACCTGCTTACTGAA
GACGAGCGAAAGGATTACGAGGAGTTGTCAAAGGTAGTCACATTCTGTCCATTCGAAAATCAGCTGTTG
TTTGCCAGGTACAATAAGATGTCTAAATTCTGTAAAAAAGAACAAGTCCTCAGCAAACTGCTGTATGCA
CTGCAAAAGAAACTTAAGGACGAAAATAGGACTAAGGAGTATATATACGTTTCATGCGTTAATAAACTC
CGGGCGAAATATGTGAGTTATTTTATTCTGAAGGAGAAGTATTATGAGAAGCAGAAAGAGTACGACATA
GAAATGGGATTTGTGGATGACAGTACGGAGAGCAAAGAAAGCATGGACAAAAGAAGGACCGAATATCCA
TTTCGAAATACTCCAGTCGCGAATGAGCTGCTGAGCAAACTTAACAATGTCCAGCAGGACATTAACGGT
TGCCTGAAGAACATAATCAACTACATATATAAGATATTTGAGCAAAACGGATACAAAGTGGTTGCACTT
GAAAACCTCGAGAATTCAAATTTCGAAAAGAAGCAAGTTTTGCCCACGATTAAAAGTCTCTTGAAATAC
CATAAGCTCGAAAATCAGAATGTGAACGATATCAAGGCCTCAGATAAGGTCAAGGAGTACATCGAAAAT
GGATATTACGAGCTGATGACGAACGAGAATAACGAAATCGTCGATGCGAAGTACACAGAAAAGGGGGCT
ATGAAGGTGAAAAACGCCAATTTTTTTAATTTGATGATGAAGTCCTTGCATTTCGCCTCAGTCAAAGAT
GAGTTTGTTCTCCTGAGTAATAATGGGAAAACACAGATAGCCTTGGTTCCTTCAGAGTTCACGTCTCAG
ATGGACTCAACTGATCATTGTCTTTATATGAAGAAAAATGATAAAGGGAAACTGGTCAAGGCCGATAAG
AAAGAGGTGCGCACGAAACAAGAAAGACACATCAACGGCCTCAACGCCGATTTTAACGCAGCTAACAAT
ATTAAATATATCGTAGAGAATGAGGTCTGGAGGGGCATTTTTTGCACCCGACCCAAGAAGACTGAATAC
AATGTCCCTAGTCTCGATACGACCAAAAAGGGGCCATCAGCTATACTGAATATGTTGAAAAAGATTGAG
GCGATTAAAGTCCTGGAAACCGAGAAA
SEQ ID NO: 25
ATGATCAAGAGCATCCAGCTGAAGGTGAAGGGAGAATGTCCTATCACAAAGGACGTGATCAACGAGTAC
AAGGAGTACTACAACAACTGCAGCGATTGGATCAAGAACAACCTGACCAGCATCACCATCGGCGAGATG
GCCAAGTTCCTGCAGTCTCTGTCCGATAAGGAAGTCGCCTACATCAGCATGGGCTTGAGCGACGAATGG
AAGGACAAACCCCTGTACCATCTGTTCACTAAGAAGTACCACACCAAGAACGCCGATAACCTGCTGTAC
TACTACATCAAGGAAAAGAACCTGGACGGCTACAAGGGCAATACCCTGAACATCAGTAACACCAGCTTC
CGGCAGTTTGGCTATTTTAAACTGGTGGTCAGCAACTACAGAACCAAGATCAGAACACTGAACTGCAAG
ATCAAGAGGAAGAAAATCGACGCCGACTCCACCAGCGAAGATATAGAGATGCAGGTGATGTACGAGATC
ATTAAGTACTCCCTGAATAAGAAGTCTGACTGGGACAACTTCATCAGCTACATCGAGAACGTGGAAAAC
CCCAACATTGACAATATCAACAGATACAAGCTGCTGCGGGAATGCTTCTGCGAGAATGAAAACATGATC
AAGAACAAGCTGGAGCTTCTGAGCGTGGAGCAGCTGAAGAAATTCGGCGGATGTATCATGAAGCCACAC
ATCAATAGCATGACCATCAACATCCAGGACTTCAAAATTGAAGAAAAGGAGAATAGCCTAGGCTTCATC
CTGCACCTGCCTCTGAACAAGAAACAGTACCAGATCGAGCTGCTGGGCAACCGGCAAATCAAGAAGGGC
ACCAAGGAGATCCACGAGACACTGGTCGACATCACAAACACACACGGCGAAAACATCGTGTTCACCATC
AAGAACGACAACCTGTACATCGTGTTCAGCTACGAGTCTGAATTCGAGAAGGAAGAGGTCAACTTCGCT
AAGACAGTGGGCCTGGACGTGAACTTCAAGCACGCCTTCTTCGTGACCAGCGAGAAAGACAACTGTCAC
CTGGACGGGTACATCAACCTGTACAAGTACCTGCTGGAACACGACGAGTTCACCAACCTCCTGACCGAA
GATGAACGGAAGGATTACGAGGAGCTGTCTAAGGTGGTGACATTCTGCCCTTTCGAGAACCAGCTGCTC
TTCGCCAGATATAACAAGATGAGCAAGTTTTGTAAAAAGGAGCAGGTGCTCAGCAAGCTACTGTACGCC
CTGCAGAAGAAGCTGAAGGACGAGAACAGAACCAAGGAATACATCTACGTGAGCTGCGTGAACAAGCTG
AGAGCCAAGTACGTGTCCTATTTCATCCTGAAGGAAAAATACTACGAGAAACAGAAAGAGTACGACATC
GAGATGGGATTTGTGGACGACAGCACCGAAAGCAAGGAATCTATGGACAAGCGCAGAACCGAGTATCCA
TTTAGAAACACCCCTGTGGCCAATGAGCTGCTGTCCAAACTGAACAACGTGCAGCAGGATATCAATGGC
TGCCTGAAAAACATCATCAACTACATTTACAAGATCTTTGAGCAAAACGGCTACAAAGTGGTGGCCCTG
GAGAACCTGGAAAACTCCAACTTCGAGAAGAAGCAAGTGCTGCCCACAATCAAGAGCCTGCTGAAGTAC
CATAAGCTGGAAAATCAGAATGTGAACGACATAAAGGCCTCTGATAAGGTGAAGGAATACATCGAAAAT
GGCTATTACGAGCTGATGACCAACGAAAATAACGAGATTGTGGACGCTAAATACACCGAGAAGGGCGCT
ATGAAAGTGAAAAACGCTAACTTTTTTAACCTGATGATGAAGAGCCTGCACTTCGCCAGCGTGAAGGAC
GAGTTCGTGCTGCTGAGCAACAACGGCAAGACACAGATCGCCCTGGTGCCCAGCGAGTTCACCAGTCAG
ATGGATTCTACAGATCACTGCCTGTACATGAAAAAGAATGATAAGGGAAAGTTAGTGAAAGCCGATAAG
AAGGAGGTGCGGACCAAACAGGAGAGACACATCAACGGCCTGAACGCTGACTTCAACGCCGCCAACAAC
ATCAAATACATCGTTGAGAATGAGGTGTGGCGGGGCATCTTCTGCACCAGACCTAAGAAAACAGAGTAT
AATGTGCCTAGCCTGGACACCACCAAGAAGGGTCCTAGCGCCATCCTGAACATGCTGAAGAAGATCGAG
GCCATCAAGGTTCTGGAAACCGAGAAG
SEQ ID NO: 26
ATGATCAAATCAATTCAGCTTAAGGTGAAGGGCGAGTGTCCCATTACTAAAGACGTCATTAACGAATAC
AAGGAGTATTACAATAACTGTAGCGACTGGATCAAGAACAATTTGACATCTATCACAATTGGGGAGATG
GCCAAATTTTTGCAGAGCCTTAGCGACAAAGAGGTCGCCTACATCTCTATGGGGCTCTCCGACGAGTGG
AAGGACAAGCCGTTGTACCACCTGTTCACAAAAAAGTATCACACAAAGAATGCAGACAATTTGCTGTAC
TACTACATTAAGGAGAAAAACCTTGATGGCTATAAAGGAAACACCCTCAACATCTCTAACACCTCTTTT
AGACAGTTCGGCTACTTTAAGCTGGTGGTGAGCAACTATAGAACCAAGATTAGGACTCTGAATTGTAAG
ATCAAAAGGAAGAAAATCGACGCTGATTCTACCTCTGAAGATATTGAGATGCAAGTCATGTATGAGATC
ATCAAGTATTCACTGAACAAGAAGAGTGATTGGGACAACTTTATTTCCTACATAGAGAACGTGGAGAAT
CCCAATATCGATAATATAAACCGATATAAGTTGCTGCGGGAGTGCTTTTGTGAAAATGAGAACATGATT
AAAAACAAGTTGGAACTCTTGTCAGTAGAACAGCTTAAAAAGTTCGGCGGCTGCATCATGAAGCCTCAT
ATCAACAGCATGACAATTAATATCCAAGATTTTAAGATCGAGGAGAAGGAAAACAGTTTGGGGTTTATT
TTGCACCTTCCACTCAATAAGAAACAGTACCAGATCGAGCTTCTGGGGAATCGGCAGATTAAGAAAGGG
ACCAAAGAGATACATGAAACTCTGGTTGATATCACAAATACCCATGGCGAAAATATTGTCTTTACCATC
AAGAATGACAACCTGTATATCGTGTTTTCTTATGAGTCCGAATTCGAGAAGGAAGAGGTGAACTTTGCC
AAGACAGTGGGGTTGGATGTCAATTTTAAGCATGCCTTCTTTGTTACCTCTGAAAAAGATAACTGCCAT
CTGGATGGATACATCAATCTCTATAAGTATCTGCTTGAGCACGATGAGTTCACAAATCTGCTGACCGAG
GACGAGAGAAAGGACTACGAAGAATTGTCCAAGGTGGTTACTTTCTGCCCATTCGAAAATCAGCTGCTG
TTCGCAAGATACAACAAAATGTCTAAATTCTGTAAAAAAGAGCAGGTCCTGTCTAAACTTCTGTATGCT
CTGCAGAAAAAACTCAAAGACGAAAACCGCACAAAAGAGTACATTTACGTAAGCTGCGTCAATAAATTG
AGGGCCAAGTATGTTTCTTATTTCATCCTTAAAGAGAAATACTACGAGAAGCAGAAGGAGTACGACATA
GAGATGGGGTTTGTAGACGATTCTACGGAGTCTAAGGAATCTATGGACAAACGGCGCACAGAGTACCCC
TTTAGGAATACCCCTGTCGCTAATGAGTTGCTCTCTAAGCTGAACAATGTCCAACAGGATATTAATGGC
TGCTTGAAGAACATAATAAATTATATTTATAAAATCTTTGAGCAGAACGGCTACAAAGTTGTTGCTCTG
GAGAACCTTGAGAACAGCAACTTTGAAAAGAAGCAGGTACTGCCTACTATTAAGTCTCTGCTGAAATAT
CATAAGCTGGAAAACCAAAATGTCAACGACATCAAGGCCTCCGACAAAGTGAAAGAGTACATCGAGAAC
GGATACTACGAGCTGATGACTAATGAGAATAACGAGATTGTCGACGCGAAATATACCGAAAAGGGCGCT
ATGAAGGTGAAGAATGCTAATTTCTTCAACTTGATGATGAAGAGCCTCCATTTTGCTTCTGTGAAGGAT
GAGTTTGTGCTGTTGAGTAACAACGGGAAGACCCAGATCGCACTGGTCCCGTCCGAGTTTACCTCCCAG
ATGGACTCTACAGATCACTGCTTGTACATGAAGAAAAACGATAAAGGTAAGTTGGTGAAAGCAGATAAG
AAAGAGGTGCGAACAAAGCAGGAGAGACATATCAACGGACTGAACGCAGATTTTAACGCTGCTAATAAT
ATTAAATATATTGTTGAGAACGAAGTGTGGCGGGGAATTTTCTGCACCAGACCGAAAAAAACAGAATAT
AATGTCCCGTCCCTCGACACTACTAAAAAAGGCCCTTCTGCGATCCTGAACATGCTTAAAAAGATTGAA
GCGATCAAGGTGTTGGAGACTGAGAAG
SEQ ID NO: 27
ATGATCAAGAGCATTCAGCTGAAAGTGAAGGGCGAGTGTCCCATCACTAAGGACGTTATCAACGAGTAC
AAGGAGTACTATAACAACTGCTCTGACTGGATTAAGAATAACCTGACCAGTATCACCATCGGCGAGATG
GCCAAGTTCCTGCAGAGCCTGAGCGACAAGGAGGTGGCCTACATCAGTATGGGGCTGTCCGATGAGTGG
AAAGACAAGCCACTGTATCATCTGTTCACCAAAAAGTACCACACCAAAAATGCCGATAACCTGCTGTAC
TATTATATCAAAGAAAAAAACCTGGACGGGTACAAAGGCAACACCCTGAACATCAGCAATACAAGTTTC
CGCCAGTTTGGATACTTCAAGCTGGTCGTGTCAAATTACAGAACCAAAATCAGAACCCTGAACTGCAAG
ATCAAAAGGAAGAAGATCGACGCTGACAGCACCAGCGAGGACATTGAGATGCAGGTGATGTACGAGATT
ATTAAATACTCCCTGAACAAGAAGAGTGATTGGGACAATTTCATTTCCTACATCGAGAATGTGGAGAAC
CCTAACATCGATAACATAAATCGGTACAAACTGCTGAGAGAGTGCTTTTGCGAGAACGAAAATATGATA
AAGAACAAGCTGGAGCTGCTCAGCGTGGAACAGCTGAAAAAATTTGGAGGCTGCATTATGAAGCCCCAC
ATCAACTCAATGACTATCAATATCCAGGACTTCAAGATCGAAGAGAAGGAGAACTCCCTGGGCTTCATT
CTGCATCTGCCACTGAATAAGAAGCAGTATCAGATCGAACTGCTGGGAAATAGGCAGATCAAGAAGGGG
ACCAAAGAGATCCACGAAACTCTGGTGGACATTACTAATACTCACGGGGAGAACATTGTGTTCACCATT
AAGAACGATAATCTGTACATCGTGTTCAGCTATGAAAGCGAGTTTGAGAAGGAAGAGGTGAACTTCGCC
AAGACCGTCGGGCTGGATGTGAATTTTAAGCACGCCTTCTTTGTGACCAGCGAAAAGGATAACTGCCAC
CTGGACGGATACATCAATCTGTACAAGTATCTGCTGGAGCACGACGAATTTACCAATCTGCTGACCGAG
GACGAGAGGAAGGACTACGAAGAGCTCTCCAAGGTGGTGACATTTTGTCCATTCGAGAACCAGCTGCTG
TTCGCCAGATACAACAAGATGTCTAAATTTTGCAAAAAAGAACAGGTCCTGAGCAAGCTGCTGTACGCC
CTGCAGAAGAAACTGAAGGACGAGAACAGAACTAAGGAGTACATCTATGTGAGCTGTGTGAATAAACTG
AGAGCAAAATACGTGTCTTACTTTATTCTGAAGGAGAAGTATTACGAGAAGCAGAAGGAATACGACATC
GAGATGGGCTTCGTGGACGATTCCACAGAGAGCAAGGAAAGCATGGATAAAAGGCGCACTGAATACCCC
TTCAGAAACACACCTGTGGCCAATGAGCTGCTGTCCAAGCTGAACAACGTGCAGCAGGACATCAACGGC
TGCCTGAAGAACATCATCAACTACATTTATAAGATCTTCGAGCAGAATGGCTACAAAGTGGTGGCACTG
GAGAACCTGGAGAACTCCAATTTTGAAAAGAAACAGGTGCTGCCCACCATTAAGAGCCTGCTTAAATAC
CATAAGCTGGAGAACCAGAACGTGAATGATATCAAAGCCAGCGATAAGGTCAAAGAGTACATCGAGAAC
GGATATTATGAGCTGATGACCAACGAGAACAATGAGATCGTGGACGCCAAATATACCGAGAAGGGCGCC
ATGAAGGTGAAGAACGCCAACTTCTTTAACCTGATGATGAAGTCCCTTCACTTTGCCTCCGTGAAGGAT
GAGTTCGTGCTGCTGAGCAACAACGGCAAGACCCAGATCGCCCTGGTGCCCTCTGAATTCACCAGCCAG
ATGGATTCAACCGACCACTGTCTCTACATGAAGAAGAACGACAAGGGCAAGCTGGTGAAGGCCGACAAG
AAAGAGGTGCGCACAAAGCAGGAGCGGCACATCAATGGCCTGAACGCTGATTTCAACGCCGCAAACAAC
ATTAAGTACATCGTGGAGAATGAGGTGTGGAGAGGTATTTTCTGCACCCGCCCCAAGAAGACCGAGTAT
AATGTGCCCTCTCTGGACACCACCAAAAAAGGCCCAAGCGCTATCCTGAATATGTTGAAGAAAATCGAG
GCCATCAAGGTGCTGGAAACCGAGAAG

A nucleic acid sequence encoding the parent polypeptide described herein may be substantially identical to a reference nucleic acid sequence, e.g., SEQ ID NO: 1, SEQ ID NO: 2, SEQ ID NO: 21, SEQ ID NO: 22, SEQ ID NO: 23, SEQ ID NO: 24, SEQ ID NO: 25, SEQ ID NO: 26, or SEQ ID NO: 27. In some embodiments, the variant polypeptide is encoded by a nucleic acid comprising a sequence having least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 99.5% sequence identity to the reference nucleic acid sequence, e.g., nucleic acid sequence encoding the parent polypeptide, e.g., SEQ ID NO: 1, SEQ ID NO: 2, SEQ ID NO: 21, SEQ ID NO: 22, SEQ ID NO: 23, SEQ ID NO: 24, SEQ ID NO: 25, SEQ ID NO: 26, or SEQ ID NO: 27. The percent identity between two such nucleic acids can be determined manually by inspection of the two optimally aligned nucleic acid sequences or by using software programs or algorithms (e.g., BLAST, ALIGN, CLUSTAL) using standard parameters. One indication that two nucleic acid sequences are substantially identical is that the nucleic acid molecules hybridize to the complementary sequence of the other under stringent conditions (e.g., within a range of medium to high stringency).

In some embodiments, the variant polypeptide is encoded by a nucleic acid sequence having at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more sequence identity, but not 100% sequence identity, to a reference nucleic acid sequence, e.g., nucleic acid sequence encoding the parent polypeptide, e.g., SEQ ID NO: 1, SEQ ID NO: 2, SEQ ID NO: 21, SEQ ID NO: 22, SEQ ID NO: 23, SEQ ID NO: 24, SEQ ID NO: 25, SEQ ID NO: 26, or SEQ ID NO: 27.

In some embodiments, the variant polypeptide of the present invention comprises a polypeptide sequence having 50%, 60%, 65%, 70%, 75%, 80%, 85%, 90%, 91%, 92%, 93%, 94%, 95%, 96%, 97%, 98%, or 99%, but not 100%, identity to SEQ ID NO: 3. In some embodiments, the variant polypeptide of the present invention comprises a polypeptide sequence having greater than 50%, 60%, 65%, 70%, 75%, 80%, 85%, 90%, 91%, 92%, 93%, 94%, 95%, 96%, 97%, 98%, or 99%, but not 100%, identity to SEQ ID NO: 3.

In some embodiments, the present invention describes a variant polypeptide having a specified degree of amino acid sequence identity to one or more reference polypeptides, e.g., a parent polypeptide, e.g., at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or even at least 99%, but not 100%, sequence identity to the amino acid sequence of SEQ ID NO: 3. Homology or identity can be determined by amino acid sequence alignment, e.g., using a program such as BLAST,

ALIGN, or CLUSTAL, as described herein. In some embodiments, the variant polypeptide maintains the amino acid changes (or at least 1, 2, 3, 4, 5 etc. of these changes) that differentiate the polypeptide from its respective parent/reference sequence.

In some embodiments, the variant polypeptide comprises an alteration at one or more (e.g., several) amino acids of a parent polypeptide, wherein at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 162, 164, 164, 165, 166, 167, 168, 169, 170, 171, 172, 173, 174, 175, 176, 177, 178, 179, 180, 181, 182, 183, 184, 185, 186, 187, 188, 189, 190, 191, 193, 194, 195, 196, 197, 198, 199, 200, or more are altered.

In some embodiments, the variant polypeptide comprises one or more of the amino acid substitutions listed in Table 2.

TABLE 2
Single Amino Acid Substitutions in Variants of SEQ ID NO: 3.
Table 2

| | Wild-Type | |
|---|---|---|
| Position | Residue | Substitutions |
| 1 | M | |
| 2 | I | R, G, A, K, Q, N, H |
| 3 | K | R, G, A, Q, N, H |
| 4 | S | R, G, A, K, Q, N, H |
| 5 | I | R, G, A, K, Q, N, H |
| 6 | Q | R, G, A, K, N, H |
| 7 | L | R, G, A, K, Q, N, H |
| 8 | K | R, G, A, Q, N, H |
| 9 | V | R, G, A, K, Q, N, H |
| 10 | K | R, G, A, Q, N, H |
| 11 | G | R, A, K, Q, N, H |
| 12 | E | R, G, A, K, Q, N, H |
| 13 | C | R, G, A, K, Q, N, H |
| 14 | P | R, G, A, K, Q, N, H |
| 15 | I | R, G, A, K, Q, N, H |
| 16 | T | R, G, A, K, Q, N, H |
| 17 | K | R, G, A, Q, N, H |
| 18 | D | R, G, A, K, Q, N, H |
| 19 | V | R, G, A, K, Q, N, H |
| 20 | I | R, G, A, K, Q, N, H |
| 21 | N | R, G, A, K, Q, H |
| 22 | E | R, G, A, K, Q, N, H |
| 23 | Y | R, G, A, K, Q, N, H |
| 24 | K | R, G, A, Q, N, H |
| 25 | E | R, G, A, K, Q, N, H |
| 26 | Y | R, G, A, K, Q, N, H |
| 27 | Y | R, G, A, K, Q, N, H |
| 28 | N | R, G, A, K, Q, H |
| 29 | N | R, G, A, K, Q, H |
| 30 | C | R, G, A, K, Q, N, H |
| 31 | S | R, G, A, K, Q, N, H |
| 32 | D | R, G, A, K, Q, N, H |
| 33 | W | R, G, A, K, Q, N, H |
| 34 | I | R, G, A, K, Q, N, H |
| 35 | K | R, G, A, Q, N, H |
| 36 | N | R, G, A, K, Q, H |
| 37 | N | R, G, A, K, Q, H |
| 38 | L | R, G, A, K, Q, N, H |
| 39 | T | R, G, A, K, Q, N, H |
| 40 | S | R, G, A, K, Q, N, H |
| 41 | I | R, G, A, K, Q, N, H |
| 42 | T | R, G, A, K, Q, N, H |
| 43 | I | R, G, A, K, Q, N, H |
| 44 | G | R, A, K, Q, N, H |
| 45 | E | R, G, A, K, Q, N, H |
| 46 | M | R, G, A, K, Q, N, H |
| 47 | A | R, G, K, Q, N, H |
| 48 | K | R, G, A, Q, N, H |
| 49 | F | R, G, A, K, Q, N, H |
| 50 | L | R, G, A, K, Q, N, H |
| 51 | Q | R, G, A, K, N, H |
| 52 | S | R, G, A, K, Q, N, H |
| 53 | L | R, G, A, K, Q, N, H |
| 54 | S | R, G, A, K, Q, N, H |
| 55 | D | R, G, A, K, Q, N, H |
| 56 | K | R, G, A, Q, N, H |
| 57 | E | R, G, A, K, Q, N, H |
| 58 | V | R, G, A, K, Q, N, H |
| 59 | A | R, G, K, Q, N, H |
| 60 | Y | R, G, A, K, Q, N, H |
| 61 | I | R, G, A, K, Q, N, H |
| 62 | S | R, G, A, K, Q, N, H |
| 63 | M | R, G, A, K, Q, N, H |
| 64 | G | R, A, K, Q, N, H |
| 65 | L | R, G, A, K, Q, N, H |
| 66 | S | R, G, A, K, Q, N, H |
| 67 | D | R, G, A, K, Q, N, H |
| 68 | E | R, G, A, K, Q, N, H |
| 69 | W | R, G, A, K, Q, N, H |
| 70 | K | R, G, A, Q, N, H |
| 71 | D | R, G, A, K, Q, N, H |
| 72 | K | R, G, A, Q, N, H |
| 73 | P | R, G, A, K, Q, N, H |
| 74 | L | R, G, A, K, Q, N, H |
| 75 | Y | R, G, A, K, Q, N, H |
| 76 | H | R, G, A, K, Q, N |
| 77 | L | R, G, A, K, Q, N, H |
| 78 | F | R, G, A, K, Q, N, H |
| 79 | T | R, G, A, K, Q, N, H |
| 80 | K | R, G, A, Q, N, H |
| 81 | K | R, G, A, Q, N, H |
| 82 | Y | R, G, A, K, Q, N, H |
| 83 | H | R, G, A, K, Q, N |
| 84 | T | R, G, A, K, Q, N, H |
| 85 | K | R, G, A, Q, N, H |
| 86 | N | R, G, A, K, Q, H |
| 87 | A | R, G, K, Q, N, H |
| 88 | D | R, G, A, K, Q, N, H |
| 89 | N | R, G, A, K, Q, H |
| 90 | L | R, G, A, K, Q, N, H |
| 91 | L | R, G, A, K, Q, N, H |
| 92 | Y | R, G, A, K, Q, N, H |
| 93 | Y | R, G, A, K, Q, N, H |
| 94 | Y | R, G, A, K, Q, N, H |
| 95 | I | R, G, A, K, Q, N, H |
| 96 | K | R, G, A, Q, N, H |
| 97 | E | R, G, A, K, Q, N, H |
| 98 | K | R, G, A, Q, N, H |
| 99 | N | R, G, A, K, Q, H |
| 100 | L | R, G, A, K, Q, N, H |
| 101 | D | R, G, A, K, Q, N, H |
| 102 | G | R, A, K, Q, N, H |
| 103 | Y | R, G, A, K, Q, N, H |
| 104 | K | R, G, A, Q, N, H |
| 105 | G | R, A, K, Q, N, H |
| 106 | N | R, G, A, K, Q, H |
| 107 | T | R, G, A, K, Q, N, H |
| 108 | L | R, G, A, K, Q, N, H |
| 109 | N | R, G, A, K, Q, H |
| 110 | I | R, G, A, K, Q, N, H |
| 111 | S | R, G, A, K, Q, N, H |
| 112 | N | R, G, A, K, Q, H |
| 113 | T | R, G, A, K, Q, N, H |
| 114 | S | R, G, A, K, Q, N, H |
| 115 | F | R, G, A, K, Q, N, H |
| 116 | R | G, A, K, Q, N, H |
| 117 | Q | R, G, A, K, N, H |
| 118 | F | R, G, A, K, Q, N, H |
| 119 | G | R, A, K, Q, N, H |
| 120 | Y | R, G, A, K, Q, N, H |
| 121 | F | R, G, A, K, Q, N, H |
| 122 | K | R, G, A, Q, N, H |
| 123 | L | R, G, A, K, Q, N, H |
| 124 | V | R, G, A, K, Q, N, H |
| 125 | V | R, G, A, K, Q, N, H |
| 126 | S | R, G, A, K, Q, N, H |
| 127 | N | R, G, A, K, Q, H |
| 128 | Y | R, G, A, K, Q, N, H |
| 129 | R | G, A, K, Q, N, H |
| 130 | T | R, G, A, K, Q, N, H |
| 131 | K | R, G, A, Q, N, H |
| 132 | I | R, G, A, K, Q, N, H |
| 133 | R | G, A, K, Q, N, H |
| 134 | T | R, G, A, K, Q, N, H |
| 135 | L | R, G, A, K, Q, N, H |
| 136 | N | R, G, A, K, Q, H |
| 137 | C | R, G, A, K, Q, N, H |
| 138 | K | R, G, A, Q, N, H |
| 139 | I | R, G, A, K, Q, N, H |
| 140 | K | R, G, A, Q, N, H |
| 141 | R | G, A, K, Q, N, H |
| 142 | K | R, G, A, Q, N, H |
| 143 | K | R, G, A, Q, N, H |
| 144 | I | R, G, A, K, Q, N, H |
| 145 | D | R, G, A, K, Q, N, H |
| 146 | A | R, G, K, Q, N, H |
| 147 | D | R, G, A, K, Q, N, H |
| 148 | S | R, G, A, K, Q, N, H |
| 149 | T | R, G, A, K, Q, N, H |
| 150 | S | R, G, A, K, Q, N, H |
| 151 | E | R, G, A, K, Q, N, H |
| 152 | D | R, G, A, K, Q, N, H |
| 153 | I | R, G, A, K, Q, N, H |
| 154 | E | R, G, A, K, Q, N, H |
| 155 | M | R, G, A, K, Q, N, H |
| 156 | Q | R, G, A, K, N, H |
| 157 | V | R, G, A, K, Q, N, H |
| 158 | M | R, G, A, K, Q, N, H |
| 159 | Y | R, G, A, K, Q, N, H |
| 160 | E | R, G, A, K, Q, N, H |
| 161 | I | R, G, A, K, Q, N, H |
| 162 | I | R, G, A, K, Q, N, H |
| 163 | K | R, G, A, Q, N, H |
| 164 | Y | R, G, A, K, Q, N, H |
| 165 | S | R, G, A, K, Q, N, H |
| 166 | L | R, G, A, K, Q, N, H |
| 167 | N | R, G, A, K, Q, H |
| 168 | K | R, G, A, Q, N, H |
| 169 | K | R, G, A, Q, N, H |
| 170 | S | R, G, A, K, Q, N, H |
| 171 | D | R, G, A, K, Q, N, H |
| 172 | W | R, G, A, K, Q, N, H |
| 173 | D | R, G, A, K, Q, N, H |
| 174 | N | R, G, A, K, Q, H |
| 175 | F | R, G, A, K, Q, N, H |
| 176 | I | R, G, A, K, Q, N, H |
| 177 | S | R, G, A, K, Q, N, H |
| 178 | Y | R, G, A, K, Q, N, H |
| 179 | I | R, G, A, K, Q, N, H |
| 180 | E | R, G, A, K, Q, N, H |
| 181 | N | R, G, A, K, Q, H |
| 182 | V | R, G, A, K, Q, N, H |
| 183 | E | R, G, A, K, Q, N, H |
| 184 | N | R, G, A, K, Q, H |
| 185 | P | R, G, A, K, Q, N, H |
| 186 | N | R, G, A, K, Q, H |
| 187 | I | R, G, A, K, Q, N, H |
| 188 | D | R, G, A, K, Q, N, H |
| 189 | N | R, G, A, K, Q, H |
| 190 | I | R, G, A, K, Q, N, H |
| 191 | N | R, G, A, K, Q, H |
| 192 | R | G, A, K, Q, N, H |
| 193 | Y | R, G, A, K, Q, N, H |
| 194 | K | R, G, A, Q, N, H |
| 195 | L | R, G, A, K, Q, N, H |
| 196 | L | R, G, A, K, Q, N, H |
| 197 | R | G, A, K, Q, N, H |
| 198 | E | R, G, A, K, Q, N, H |
| 199 | C | R, G, A, K, Q, N, H |
| 200 | F | R, G, A, K, Q, N, H |
| 201 | C | R, G, A, K, Q, N, H |
| 202 | E | R, G, A, K, Q, N, H |
| 203 | N | R, G, A, K, Q, H |
| 204 | E | R, G, A, K, Q, N, H |
| 205 | N | R, G, A, K, Q, H |
| 206 | M | R, G, A, K, Q, N, H |
| 207 | I | R, G, A, K, Q, N, H |
| 208 | K | R, G, A, Q, N, H |
| 209 | N | R, G, A, K, Q, H |
| 210 | K | R, G, A, Q, N, H |
| 211 | L | R, G, A, K, Q, N, H |
| 212 | E | R, G, A, K, Q, N, H |
| 213 | L | R, G, A, K, Q, N, H |
| 214 | L | R, G, A, K, Q, N, H |
| 215 | S | R, G, A, K, Q, N, H |
| 216 | V | R, G, A, K, Q, N, H |
| 217 | E | R, G, A, K, Q, N, H |
| 218 | Q | R, G, A, K, N, H |
| 219 | L | R, G, A, K, Q, N, H |
| 220 | K | R, G, A, Q, N, H |
| 221 | K | R, G, A, Q, N, H |
| 222 | F | R, G, A, K, Q, N, H |
| 223 | G | R, A, K, Q, N, H |
| 224 | G | R, A, K, Q, N, H |
| 225 | C | R, G, A, K, Q, N, H |
| 226 | I | R, G, A, K, Q, N, H |
| 227 | M | R, G, A, K, Q, N, H |
| 228 | K | R, G, A, Q, N, H |
| 229 | P | R, G, A, K, Q, N, H |
| 230 | H | R, G, A, K, Q, N |
| 231 | I | R, G, A, K, Q, N, H |
| 232 | N | R, G, A, K, Q, H |
| 233 | S | R, G, A, K, Q, N, H |
| 234 | M | R, G, A, K, Q, N, H |
| 235 | T | R, G, A, K, Q, N, H |
| 236 | I | R, G, A, K, Q, N, H |
| 237 | N | R, G, A, K, Q, H |
| 238 | I | R, G, A, K, Q, N, H |
| 239 | Q | R, G, A, K, N, H |
| 240 | D | R, G, A, K, Q, N, H |
| 241 | F | R, G, A, K, Q, N, H |
| 242 | K | R, G, A, Q, N, H |
| 243 | I | R, G, A, K, Q, N, H |
| 244 | E | R, G, A, K, Q, N, H |
| 245 | E | R, G, A, K, Q, N, H |
| 246 | K | R, G, A, Q, N, H |
| 247 | E | R, G, A, K, Q, N, H |
| 248 | N | R, G, A, K, Q, H |
| 249 | S | R, G, A, K, Q, N, H |
| 250 | L | R, G, A, K, Q, N, H |
| 251 | G | R, A, K, Q, N, H |
| 252 | F | R, G, A, K, Q, N, H |
| 253 | I | R, G, A, K, Q, N, H |
| 254 | L | R, G, A, K, Q, N, H |
| 255 | H | R, G, A, K, Q, N |
| 256 | L | R, G, A, K, Q, N, H |
| 257 | P | R, G, A, K, Q, N, H |
| 258 | L | R, G, A, K, Q, N, H |
| 259 | N | R, G, A, K, Q, H |
| 260 | K | R, G, A, Q, N, H |
| 261 | K | R, G, A, Q, N, H |
| 262 | Q | R, G, A, K, N, H |
| 263 | Y | R, G, A, K, Q, N, H |
| 264 | Q | R, G, A, K, N, H |
| 265 | I | R, G, A, K, Q, N, H |
| 266 | E | R, G, A, K, Q, N, H |
| 267 | L | R, G, A, K, Q, N, H |
| 268 | L | R, G, A, K, Q, N, H |
| 269 | G | R, A, K, Q, N, H |
| 270 | N | R, G, A, K, Q, H |
| 271 | R | G, A, K, Q, N, H |
| 272 | Q | R, G, A, K, N, H |
| 273 | I | R, G, A, K, Q, N, H |
| 274 | K | R, G, A, Q, N, H |
| 275 | K | R, G, A, Q, N, H |
| 276 | G | R, A, K, Q, N, H |
| 277 | T | R, G, A, K, Q, N, H |
| 278 | K | R, G, A, Q, N, H |
| 279 | E | R, G, A, K, Q, N, H |
| 280 | I | R, G, A, K, Q, N, H |
| 281 | H | R, G, A, K, Q, N |
| 282 | E | R, G, A, K, Q, N, H |
| 283 | T | R, G, A, K, Q, N, H |
| 284 | L | R, G, A, K, Q, N, H |
| 285 | V | R, G, A, K, Q, N, H |
| 286 | D | R, G, A, K, Q, N, H |
| 287 | I | R, G, A, K, Q, N, H |
| 288 | T | R, G, A, K, Q, N, H |
| 289 | N | R, G, A, K, Q, H |
| 290 | T | R, G, A, K, Q, N, H |
| 291 | H | R, G, A, K, Q, N, |
| 292 | G | R, A, K, Q, N, H |
| 293 | E | R, G, A, K, Q, N, H |
| 294 | N | R, G, A, K, Q, H |
| 295 | I | R, G, A, K, Q, N, H |
| 296 | V | R, G, A, K, Q, N, H |
| 297 | F | R, G, A, K, Q, N, H |
| 298 | T | R, G, A, K, Q, N, H |
| 299 | I | R, G, A, K, Q, N, H |
| 300 | K | R, G, A, Q, N, H |
| 301 | N | R, G, A, K, Q, H |
| 302 | D | R, G, A, K, Q, N, H |
| 303 | N | R, G, A, K, Q, H |
| 304 | L | R, G, A, K, Q, N, H |
| 305 | Y | R, G, A, K, Q, N, H |
| 306 | I | R, G, A, K, Q, N, H |
| 307 | V | R, G, A, K, Q, N, H |
| 308 | F | R, G, A, K, Q, N, H |
| 309 | S | R, G, A, K, Q, N, H |
| 310 | Y | R, G, A, K, Q, N, H |
| 311 | E | R, G, A, K, Q, N, H |
| 312 | S | R, G, A, K, Q, N, H |
| 313 | E | R, G, A, K, Q, N, H |
| 314 | F | R, G, A, K, Q, N, H |
| 315 | E | R, G, A, K, Q, N, H |
| 316 | K | R, G, A, Q, N, H |
| 317 | E | R, G, A, K, Q, N, H |
| 318 | E | R, G, A, K, Q, N, H |
| 319 | V | R, G, A, K, Q, N, H |
| 320 | N | R, G, A, K, Q, H |
| 321 | F | R, G, A, K, Q, N, H |
| 322 | A | R, G, K, Q, N, H |
| 323 | K | R, G, A, Q, N, H |
| 324 | T | R, G, A, K, Q, N, H |
| 325 | V | R, G, A, K, Q, N, H |
| 326 | G | R, A, K, Q, N, H |
| 327 | L | R, G, A, K, Q, N, H |
| 328 | D | A, C, E, F, G, H, I, K, L, M, |
| | | N, P, Q, R, S, T, V, W, Y |
| 329 | V | R, G, A, K, Q, N, H |
| 330 | N | R, G, A, K, Q, H |
| 331 | F | R, G, A, K, Q, N, H |
| 332 | K | R, G, A, Q, N, H |
| 333 | H | R, G, A, K, Q, N |
| 334 | A | R, G, K, Q, N, H |
| 335 | F | R, G, A, K, Q, N, H |
| 336 | F | R, G, A, K, Q, N, H |
| 337 | V | R, G, A, K, Q, N, H |
| 338 | T | R, G, A, K, Q, N, H |
| 339 | S | R, G, A, K, Q, N, H |
| 340 | E | R, G, A, K, Q, N, H |
| 341 | K | R, G, A, Q, N, H |
| 342 | D | R, G, A, K, Q, N, H |
| 343 | N | R, G, A, K, Q, H |
| 344 | C | R, G, A, K, Q, N, H |
| 345 | H | R, G, A, K, Q, N |
| 346 | L | R, G, A, K, Q, N, H |
| 347 | D | R, G, A, K, Q, N, H |
| 348 | G | R, A, K, Q, N, H |
| 349 | Y | R, G, A, K, Q, N, H |
| 350 | I | R, G, A, K, Q, N, H |
| 351 | N | R, G, A, K, Q, H |
| 352 | L | R, G, A, K, Q, N, H |
| 353 | Y | R, G, A, K, Q, N, H |
| 354 | K | R, G, A, Q, N, H |
| 355 | Y | R, G, A, K, Q, N, H |
| 356 | L | R, G, A, K, Q, N, H |
| 357 | L | R, G, A, K, Q, N, H |
| 358 | E | R, G, A, K, Q, N, H |
| 359 | H | R, G, A, K, Q, N |
| 360 | D | R, G, A, K, Q, N, H |
| 361 | E | R, G, A, K, Q, N, H |
| 362 | F | R, G, A, K, Q, N, H |
| 363 | T | R, G, A, K, Q, N, H |
| 364 | N | R, G, A, K, Q, H |
| 365 | L | R, G, A, K, Q, N, H |
| 366 | L | R, G, A, K, Q, N, H |
| 367 | T | R, G, A, K, Q, N, H |
| 368 | E | R, G, A, K, Q, N, H |
| 369 | D | R, G, A, K, Q, N, H |
| 370 | E | R, G, A, K, Q, N, H |
| 371 | R | G, A, K, Q, N, H |
| 372 | K | R, G, A, Q, N, H |
| 373 | D | R, G, A, K, Q, N, H |
| 374 | Y | R, G, A, K, Q, N, H |
| 375 | E | R, G, A, K, Q, N, H |
| 376 | E | R, G, A, K, Q, N, H |
| 377 | L | R, G, A, K, Q, N, H |
| 378 | S | R, G, A, K, Q, N, H |
| 379 | K | R, G, A, Q, N, H |
| 380 | V | R, G, A, K, Q, N, H |
| 381 | V | R, G, A, K, Q, N, H |
| 382 | T | R, G, A, K, Q, N, H |
| 383 | F | R, G, A, K, Q, N, H |
| 384 | C | R, G, A, K, Q, N, H |
| 385 | P | R, G, A, K, Q, N, H |
| 386 | F | R, G, A, K, Q, N, H |
| 387 | E | R, G, A, K, Q, N, H |
| 388 | N | R, G, A, K, Q, H |
| 389 | Q | R, G, A, K, N, H |
| 390 | L | R, G, A, K, Q, N, H |
| 391 | L | R, G, A, K, Q, N, H |
| 392 | F | R, G, A, K, Q, N, H |
| 393 | A | R, G, K, Q, N, H |
| 394 | R | G, A, K, Q, N, H |
| 395 | Y | R, G, A, K, Q, N, H |
| 396 | N | R, G, A, K, Q, H |
| 397 | K | R, G, A, Q, N, H |
| 398 | M | R, G, A, K, Q, N, H |
| 399 | S | R, G, A, K, Q, N, H |
| 400 | K | R, G, A, Q, N, H |
| 401 | F | R, G, A, K, Q, N, H |
| 402 | C | R, G, A, K, Q, N, H |
| 403 | K | R, G, A, Q, N, H |
| 404 | K | R, G, A, Q, N, H |
| 405 | E | R, G, A, K, Q, N, H |
| 406 | Q | R, G, A, K, N, H |
| 407 | V | R, G, A, K, Q, N, H |
| 408 | L | R, G, A, K, Q, N, H |
| 409 | S | R, G, A, K, Q, N, H |
| 410 | K | R, G, A, Q, N, H |
| 411 | L | R, G, A, K, Q, N, H |
| 412 | L | R, G, A, K, Q, N, H |
| 413 | Y | R, G, A, K, Q, N, H |
| 414 | A | R, G, K, Q, N, H |
| 415 | L | R, G, A, K, Q, N, H |
| 416 | Q | R, G, A, K, N, H |
| 417 | K | R, G, A, Q, N, H |
| 418 | K | R, G, A, Q, N, H |
| 419 | L | R, G, A, K, Q, N, H |
| 420 | K | R, G, A, Q, N, H |
| 421 | D | R, G, A, K, Q, N, H |
| 422 | E | R, G, A, K, Q, N, H |
| 423 | N | R, G, A, K, Q, H |
| 424 | T | R, G, A, K, Q, N, H |
| 425 | R | G, A, K, Q, N, H |
| 426 | K | R, G, A, Q, N, H |
| 427 | E | R, G, A, K, Q, N, H |
| 428 | Y | R, G, A, K, Q, N, H |
| 429 | I | R, G, A, K, Q, N, H |
| 430 | Y | R, G, A, K, Q, N, H |
| 431 | V | R, G, A, K, Q, N, H |
| 432 | S | R, G, A, K, Q, N, H |
| 433 | C | R, G, A, K, Q, N, H |
| 434 | V | R, G, A, K, Q, N, H |
| 435 | N | R, G, A, K, Q, H |
| 436 | K | R, G, A, Q, N, H |
| 437 | L | R, G, A, K, Q, N, H |
| 438 | R | G, A, K, Q, N, H |
| 439 | A | R, G, K, Q, N, H |
| 440 | K | R, G, A, Q, N, H |
| 441 | Y | R, G, A, K, Q, N, H |
| 442 | V | R, G, A, K, Q, N, H |
| 443 | S | R, G, A, K, Q, N, H |
| 444 | Y | R, G, A, K, Q, N, H |
| 445 | F | R, G, A, K, Q, N, H |
| 446 | I | R, G, A, K, Q, N, H |
| 447 | L | R, G, A, K, Q, N, H |
| 448 | K | R, G, A, Q, N, H |
| 449 | E | R, G, A, K, Q, N, H |
| 450 | K | R, G, A, Q, N, H |
| 451 | Y | R, G, A, K, Q, N, H |
| 452 | Y | R, G, A, K, Q, N, H |
| 453 | E | R, G, A, K, Q, N, H |
| 454 | K | R, G, A, Q, N, H |
| 455 | Q | R, G, A, K, N, H |
| 456 | K | R, G, A, Q, N, H |
| 457 | E | R, G, A, K, Q, N, H |
| 458 | Y | R, G, A, K, Q, N, H |
| 459 | D | R, G, A, K, Q, N, H |
| 460 | I | R, G, A, K, Q, N, H |
| 461 | E | R, G, A, K, Q, N, H |
| 462 | M | R, G, A, K, Q, N, H |
| 463 | G | R, A, K, Q, N, H |
| 464 | F | R, G, A, K, Q, N, H |
| 465 | V | R, G, A, K, Q, N, H |
| 466 | D | R, G, A, K, Q, N, H |
| 467 | D | R, G, A, K, Q, N, H |
| 468 | S | R, G, A, K, Q, N, H |
| 469 | T | R, G, A, K, Q, N, H |
| 470 | E | R, G, A, K, Q, N, H |
| 471 | S | R, G, A, K, Q, N, H |
| 472 | K | R, G, A, Q, N, H |
| 473 | E | R, G, A, K, Q, N, H |
| 474 | S | R, G, A, K, Q, N, H |
| 475 | M | R, G, A, K, Q, N, H |
| 476 | D | R, G, A, K, Q, N, H |
| 477 | K | R, G, A, Q, N, H |
| 478 | R | G, A, K, Q, N, H |
| 479 | R | G, A, K, Q, N, H |
| 480 | T | R, G, A, K, Q, N, H |
| 481 | E | R, G, A, K, Q, N, H |
| 482 | Y | R, G, A, K, Q, N, H |
| 483 | P | R, G, A, K, Q, N, H |
| 484 | F | R, G, A, K, Q, N, H |
| 485 | R | G, A, K, Q, N, H |
| 486 | N | R, G, A, K, Q, H |
| 487 | T | R, G, A, K, Q, N, H |
| 488 | P | R, G, A, K, Q, N, H |
| 489 | V | R, G, A, K, Q, N, H |
| 490 | A | R, G, K, Q, N, H |
| 491 | N | R, G, A, K, Q, H |
| 492 | E | R, G, A, K, Q, N, H |
| 493 | L | R, G, A, K, Q, N, H |
| 494 | L | R, G, A, K, Q, N, H |
| 495 | S | R, G, A, K, Q, N, H |
| 496 | K | R, G, A, Q, N, H |
| 497 | L | R, G, A, K, Q, N, H |
| 498 | N | R, G, A, K, Q, H |
| 499 | N | R, G, A, K, Q, H |
| 500 | V | R, G, A, K, Q, N, H |
| 501 | Q | R, G, A, K, N, H |
| 502 | Q | R, G, A, K, N, H |
| 503 | D | R, G, A, K, Q, N, H |
| 504 | I | R, G, A, K, Q, N, H |
| 505 | N | R, G, A, K, Q, H |
| 506 | G | R, A, K, Q, N, H |
| 507 | C | R, G, A, K, Q, N, H |
| 508 | L | R, G, A, K, Q, N, H |
| 509 | K | R, G, A, Q, N, H |
| 510 | N | R, G, A, K, Q, H |
| 511 | I | R, G, A, K, Q, N, H |
| 512 | I | R, G, A, K, Q, N, H |
| 513 | N | R, G, A, K, Q, H |
| 514 | Y | R, G, A, K, Q, N, H |
| 515 | I | R, G, A, K, Q, N, H |
| 516 | Y | R, G, A, K, Q, N, H |
| 517 | K | R, G, A, Q, N, H |
| 518 | I | R, G, A, K, Q, N, H |
| 519 | F | R, G, A, K, Q, N, H |
| 520 | E | R, G, A, K, Q, N, H |
| 521 | Q | R, G, A, K, N, H |
| 522 | N | R, G, A, K, Q, H |
| 523 | G | R, A, K, Q, N, H |
| 524 | Y | R, G, A, K, Q, N, H |
| 525 | K | R, G, A, Q, N, H |
| 526 | V | R, G, A, K, Q, N, H |
| 527 | V | R, G, A, K, Q, N, H |
| 528 | A | R, G, K, Q, N, H |
| 529 | L | R, G, A, K, Q, N, H |
| 530 | E | A, C, D, F, G, H, I, K, L, M, |
| | | N, P, Q, R, S, T, V, W, Y |
| 531 | N | R, G, A, K, Q, H |
| 532 | L | R, G, A, K, Q, N, H |
| 533 | E | R, G, A, K, Q, N, H |
| 534 | N | R, G, A, K, Q, H |
| 535 | S | R, G, A, K, Q, N, H |
| 536 | N | R, G, A, K, Q, H |
| 537 | F | R, G, A, K, Q, N, H |
| 538 | E | R, G, A, K, Q, N, H |
| 539 | K | R, G, A, Q, N, H |
| 540 | K | R, G, A, Q, N, H |
| 541 | Q | R, G, A, K, N, H |
| 542 | V | R, G, A, K, Q, N, H |
| 543 | L | R, G, A, K, Q, N, H |
| 544 | P | R, G, A, K, Q, N, H |
| 545 | T | R, G, A, K, Q, N, H |
| 546 | I | R, G, A, K, Q, N, H |
| 547 | K | R, G, A, Q, N, H |
| 548 | S | R, G, A, K, Q, N, H |
| 549 | L | R, G, A, K, Q, N, H |
| 550 | L | R, G, A, K, Q, N, H |
| 551 | K | R, G, A, Q, N, H |
| 552 | Y | R, G, A, K, Q, N, H |
| 553 | H | R, G, A, K, Q, N |
| 554 | K | R, G, A, Q, N, H |
| 555 | L | R, G, A, K, Q, N, H |
| 556 | E | R, G, A, K, Q, N, H |
| 557 | N | R, G, A, K, Q, H |
| 558 | Q | R, G, A, K, N, H |
| 559 | N | R, G, A, K, Q, H |
| 560 | V | R, G, A, K, Q, N, H |
| 561 | N | R, G, A, K, Q, H |
| 562 | D | R, G, A, K, Q, N, H |
| 563 | I | R, G, A, K, Q, N, H |
| 564 | K | R, G, A, Q, N, H |
| 565 | A | R, G, K, Q, N, H |
| 566 | S | R, G, A, K, Q, N, H |
| 567 | D | R, G, A, K, Q, N, H |
| 568 | K | R, G, A, Q, N, H |
| 569 | V | R, G, A, K, Q, N, H |
| 570 | K | R, G, A, Q, N, H |
| 571 | E | R, G, A, K, Q, N, H |
| 572 | Y | R, G, A, K, Q, N, H |
| 573 | I | R, G, A, K, Q, N, H |
| 574 | E | R, G, A, K, Q, N, H |
| 575 | N | R, G, A, K, Q, H |
| 576 | G | R, A, K, Q, N, H |
| 577 | Y | R, G, A, K, Q, N, H |
| 578 | Y | R, G, A, K, Q, N, H |
| 579 | E | R, G, A, K, Q, N, H |
| 580 | L | R, G, A, K, Q, N, H |
| 581 | M | R, G, A, K, Q, N, H |
| 582 | T | R, G, A, K, Q, N, H |
| 583 | N | R, G, A, K, Q, H |
| 584 | E | R, G, A, K, Q, N, H |
| 585 | N | R, G, A, K, Q, H |
| 586 | N | R, G, A, K, Q, H |
| 587 | E | R, G, A, K, Q, N, H |
| 588 | I | R, G, A, K, Q, N, H |
| 589 | V | R, G, A, K, Q, N, H |
| 590 | D | R, G, A, K, Q, N, H |
| 591 | A | R, G, K, Q, N, H |
| 592 | K | R, G, A, Q, N, H |
| 593 | Y | R, G, A, K, Q, N, H |
| 594 | T | R, G, A, K, Q, N, H |
| 595 | E | R, G, A, K, Q, N, H |
| 596 | K | R, G, A, Q, N, H |
| 597 | G | R, A, K, Q, N, H |
| 598 | A | R, G, K, Q, N, H |
| 599 | M | R, G, A, K, Q, N, H |
| 600 | K | R, G, A, Q, N, H |
| 601 | V | R, G, A, K, Q, N, H |
| 602 | K | R, G, A, Q, N, H |
| 603 | N | R, G, A, K, Q, H |
| 604 | A | R, G, K, Q, N, H |
| 605 | N | R, G, A, K, Q, H |
| 606 | F | R, G, A, K, Q, N, H |
| 607 | F | R, G, A, K, Q, N, H |
| 608 | N | R, G, A, K, Q, H |
| 609 | L | R, G, A, K, Q, N, H |
| 610 | M | R, G, A, K, Q, N, H |
| 611 | M | R, G, A, K, Q, N, H |
| 612 | K | R, G, A, Q, N, H |
| 613 | S | R, G, A, K, Q, N, H |
| 614 | L | R, G, A, K, Q, N, H |
| 615 | H | R, G, A, K, Q, N |
| 616 | F | R, G, A, K, Q, N, H |
| 617 | A | R, G, K, Q, N, H |
| 618 | S | R, G, A, K, Q, N, H |
| 619 | V | R, G, A, K, Q, N, H |
| 620 | K | R, G, A, Q, N, H |
| 621 | D | R, G, A, K, Q, N, H |
| 622 | E | R, G, A, K, Q, N, H |
| 623 | F | R, G, A, K, Q, N, H |
| 624 | V | R, G, A, K, Q, N, H |
| 625 | L | R, G, A, K, Q, N, H |
| 626 | L | R, G, A, K, Q, N, H |
| 627 | S | R, G, A, K, Q, N, H |
| 628 | N | R, G, A, K, Q, H |
| 629 | N | R, G, A, K, Q, H |
| 630 | G | R, A, K, Q, N, H |
| 631 | K | R, G, A, Q, N, H |
| 632 | T | R, G, A, K, Q, N, H |
| 633 | Q | R, G, A, K, N, H |
| 634 | I | R, G, A, K, Q, N, H |
| 635 | A | R, G, K, Q, N, H |
| 636 | L | R, G, A, K, Q, N, H |
| 637 | V | R, G, A, K, Q, N, H |
| 638 | P | R, G, A, K, Q, N, H |
| 639 | S | R, G, A, K, Q, N, H |
| 640 | E | R, G, A, K, Q, N, H |
| 641 | F | R, G, A, K, Q, N, H |
| 642 | T | R, G, A, K, Q, N, H |
| 643 | S | R, G, A, K, Q, N, H |
| 644 | Q | R, G, A, K, N, H |
| 645 | M | R, G, A, K, Q, N, H |
| 646 | D | R, G, A, K, Q, N, H |
| 647 | S | R, G, A, K, Q, N, H |
| 648 | T | R, G, A, K, Q, N, H |
| 649 | D | R, G, A, K, Q, N, H |
| 650 | H | R, G, A, K, Q, N |
| 651 | C | R, G, A, K, Q, N, H |
| 652 | L | R, G, A, K, Q, N, H |
| 653 | Y | R, G, A, K, Q, N, H |
| 654 | M | R, G, A, K, Q, N, H |
| 655 | K | R, G, A, Q, N, H |
| 656 | K | R, G, A, Q, N, H |
| 657 | N | R, G, A, K, Q, H |
| 658 | D | R, G, A, K, Q, N, H |
| 659 | K | R, G, A, Q, N, H |
| 660 | G | R, A, K, Q, N, H |
| 661 | K | R, G, A, Q, N, H |
| 662 | L | R, G, A, K, Q, N, H |
| 663 | V | R, G, A, K, Q, N, H |
| 664 | K | R, G, A, Q, N, H |
| 665 | A | R, G, K, Q, N, H |
| 666 | D | R, G, A, K, Q, N, H |
| 667 | K | R, G, A, Q, N, H |
| 668 | K | R, G, A, Q, N, H |
| 669 | E | R, G, A, K, Q, N, H |
| 670 | V | R, G, A, K, Q, N, H |
| 671 | R | G, A, K, Q, N, H |
| 672 | T | R, G, A, K, Q, N, H |
| 673 | K | R, G, A, Q, N, H |
| 674 | Q | R, G, A, K, N, H |
| 675 | E | R, G, A, K, Q, N, H |
| 676 | R | G, A, K, Q, N, H |
| 677 | H | R, G, A, K, Q, N |
| 678 | I | R, G, A, K, Q, N, H |
| 679 | N | R, G, A, K, Q, N, H |
| 680 | G | R, A, K, Q, N, H |
| 681 | L | R, G, A, K, Q, N, H |
| 682 | N | R, G, A, K, Q, H |
| 683 | A | R, G, K, Q, N, H |
| 684 | D | R, G, A, K, Q, N, H |
| 685 | F | R, G, A, K, Q, N, H |
| 686 | N | R, G, A, K, Q, H |
| 687 | A | R, G, K, Q, N, H |
| 688 | A | R, G, K, Q, N, H |
| 689 | N | R, G, A, K, Q, H |
| 690 | N | R, G, A, K, Q, H |
| 691 | I | R, G, A, K, Q, N, H |
| 692 | K | R, G, A, Q, N, H |
| 693 | Y | R, G, A, K, Q, N, H |
| 694 | I | R, G, A, K, Q, N, H |
| 695 | V | R, G, A, K, Q, N, H |
| 696 | E | R, G, A, K, Q, N, H |
| 697 | N | R, G, A, K, Q, H |
| 698 | E | R, G, A, K, Q, N, H |
| 699 | V | R, G, A, K, Q, N, H |
| 700 | W | R, G, A, K, Q, N, H |
| 701 | R | G, A, K, Q, N, H |
| 702 | G | R, A, K, Q, N, H |
| 703 | I | R, G, A, K, Q, N, H |
| 704 | F | R, G, A, K, Q, N, H |
| 705 | C | R, G, A, K, Q, N, H |
| 706 | T | R, G, A, K, Q, N, H |
| 707 | R | G, A, K, Q, N, H |
| 708 | P | R, G, A, K, Q, N, H |
| 709 | K | R, G, A, Q, N, H |
| 710 | K | R, G, A, Q, N, H |
| 711 | T | R, G, A, K, Q, N, H |
| 712 | E | R, G, A, K, Q, N, H |
| 713 | Y | R, G, A, K, Q, N, H |
| 714 | N | R, G, A, K, Q, H |
| 715 | V | R, G, A, K, Q, N, H |
| 716 | P | R, G, A, K, Q, N, H |
| 717 | S | R, G, A, K, Q, N, H |
| 718 | L | R, G, A, K, Q, N, H |
| 719 | D | R, G, A, K, Q, N, H |
| 720 | T | R, G, A, K, Q, N, H |
| 721 | T | R, G, A, K, Q, N, H |
| 722 | K | R, G, A, Q, N, H |
| 723 | K | R, G, A, Q, N, H |
| 724 | G | R, A, K, Q, N, H |
| 725 | P | R, G, A, K, Q, N, H |
| 726 | S | R, G, A, K, Q, N, H |
| 727 | A | R, G, K, Q, N, H |
| 728 | I | R, G, A, K, Q, N, H |
| 729 | L | R, G, A, K, Q, N, H |
| 730 | N | R, G, A, K, Q, H |
| 731 | M | R, G, A, K, Q, N, H |
| 732 | L | R, G, A, K, Q, N, H |
| 733 | K | R, G, A, Q, N, H |
| 734 | K | R, G, A, Q, N, H |
| 735 | I | R, G, A, K, Q, N, H |
| 736 | E | R, G, A, K, Q, N, H |
| 737 | A | R, G, K, Q, N, H |
| 738 | I | R, G, A, K, Q, N, H |
| 739 | K | R, G, A, Q, N, H |
| 740 | V | R, G, A, K, Q, N, H |
| 741 | L | R, G, A, K, Q, N, H |
| 742 | E | R, G, A, K, Q, N, H |
| 743 | T | R, G, A, K, Q, N, H |
| 744 | E | R, G, A, K, Q, N, H |
| 745 | K | R, G, A, Q, N, H |

In some embodiments, the variant polypeptide comprises an alteration that increases interactions of the variant polypeptide to the RNA guide. In some embodiments, the alteration that increases interactions with the RNA guide is an arginine, lysine, glutamine, asparagine, or histidine substitution. In some embodiments, the variant polypeptide comprises an alteration that increases interactions of the variant polypeptide to the target nucleic acid. In some embodiments, the alteration that increases interactions with the target nucleic acid is an arginine, lysine, glutamine, asparagine, or histidine substitution. In some embodiments, the variant polypeptide comprises an alanine substitution. In some embodiments, the variant polypeptide comprises a glycine substitution.

In some embodiments, the variant polypeptide comprises a substitution at P14, E311, D32, 161, G223, N109, and/or D719 relative to the parent polypeptide of SEQ ID NO: 3.

In some embodiments, the variant polypeptide comprises an amino acid sequence having at least 95%, 96%, 97%, 98%, or 99% identity to SEQ ID NO: 3 and comprising a substitution at position P14 (e.g., a P14R substitution) relative to SEQ ID NO: 3. In some embodiments, the variant polypeptide comprises an amino acid sequence having one or more sequence alterations (e.g., substitutions, insertions, or deletions, or any combination thereof) at up to 1, 2, 3, 4, 5, 10, 15, 20, 25, 30, or 35 amino acid positions of SEQ ID NO: 3, wherein one of the sequence alterations comprises a substitution at position P14 (e.g., a P14R substitution) relative to SEQ ID NO: 3.

In some embodiments, the variant polypeptide comprises an amino acid sequence having at least 95%, 96%, 97%, 98%, or 99% identity to SEQ ID NO: 3 and comprising a substitution at position E311 (e.g., an E311R substitution) relative to SEQ ID NO: 3. In some embodiments, the variant polypeptide comprises an amino acid sequence having one or more sequence alterations (e.g., substitutions, insertions, or deletions, or any combination thereof) at up to 1, 2, 3, 4, 5, 10, 15, 20, 25, 30, or 35 amino acid positions of SEQ ID NO: 3, wherein one of the sequence alterations comprises a substitution at position E311 (e.g., an E311R substitution) relative to SEQ ID NO: 3.

In some embodiments, the variant polypeptide comprises an amino acid sequence having at least 95%, 96%, 97%, 98%, or 99% identity to SEQ ID NO: 3 and comprising a substitution at position D32 (e.g., a D32R substitution) relative to SEQ ID NO: 3. In some embodiments, the variant polypeptide comprises an amino acid sequence having one or more sequence alterations (e.g., substitutions, insertions, or deletions, or any combination thereof) at up to 1, 2, 3, 4, 5, 10, 15, 20, 25, 30, or 35 amino acid positions of SEQ ID NO: 3, wherein one of the sequence alterations comprises a substitution at position D32 (e.g., a D32R substitution) relative to SEQ ID NO: 3.

In some embodiments, the variant polypeptide comprises an amino acid sequence having at least 95%, 96%, 97%, 98%, or 99% identity to SEQ ID NO: 3 and comprising a substitution at position 161 (e.g., an 161R substitution) relative to SEQ ID NO: 3. In some embodiments, the variant polypeptide comprises an amino acid sequence having one or more sequence alterations (e.g., substitutions, insertions, or deletions, or any combination thereof) at up to 1, 2, 3, 4, 5, 10, 15, 20, 25, 30, or 35 amino acid positions of SEQ ID NO: 3, wherein one of the sequence alterations comprises a substitution at position 161 (e.g., an 161R substitution) relative to SEQ ID NO: 3.

In some embodiments, the variant polypeptide comprises an amino acid sequence having at least 95%, 96%, 97%, 98%, or 99% identity to SEQ ID NO: 3 and comprising a substitution at position G223 (e.g., a G223R substitution) relative to SEQ ID NO: 3. In some embodiments, the variant polypeptide comprises an amino acid sequence having one or more sequence alterations (e.g., substitutions, insertions, or deletions, or any combination thereof) at up to 1, 2, 3, 4, 5, 10, 15, 20, 25, 30, or 35 amino acid positions of SEQ ID NO: 3, wherein one of the sequence alterations comprises a substitution at position G223 (e.g., a G223R substitution) relative to SEQ ID NO: 3.

In some embodiments, the variant polypeptide comprises an amino acid sequence having at least 95%, 96%, 97%, 98%, or 99% identity to SEQ ID NO: 3 and comprising a substitution at position N109 (e.g., an N109R substitution) relative to SEQ ID NO: 3. In some embodiments, the variant polypeptide comprises an amino acid sequence having one or more sequence alterations (e.g., substitutions, insertions, or deletions, or any combination thereof) at up to 1, 2, 3, 4, 5, 10, 15, 20, 25, 30, or 35 amino acid positions of SEQ ID NO: 3, wherein one of the sequence alterations comprises a substitution at position N109 (e.g., an N109R substitution) relative to SEQ ID NO: 3.

In some embodiments, the variant polypeptide comprises an amino acid sequence having at least 95%, 96%, 97%, 98%, or 99% identity to SEQ ID NO: 3 and comprising a substitution at position D719 (e.g., a D719R substitution) relative to SEQ ID NO: 3. In some embodiments, the variant polypeptide comprises an amino acid sequence having one or more sequence alterations (e.g., substitutions, insertions, or deletions, or any combination thereof) at up to 1, 2, 3, 4, 5, 10, 15, 20, 25, 30, or 35 amino acid positions of SEQ ID NO: 3, wherein one of the sequence alterations comprises a substitution at position D719 (e.g., a D719R substitution) relative to SEQ ID NO: 3.

In some embodiments, the variant polypeptide comprises the substitution P14R, E311R, D32R. 161R. G223R. N109R, and/or D719R relative to the parent polypeptide of SEQ ID NO: 3. In some embodiments, the variant polypeptide comprises the substitution P14R and one, two, three, or four additional substitutions relative to the parent polypeptide of SEQ ID NO: 3. In some embodiments, the variant polypeptide comprises the substitution E311R and one, two, three, or four additional substitutions relative to the parent polypeptide of SEQ ID NO: 3. In some embodiments, the variant polypeptide comprises the substitution D32R and one, two, three, or four additional substitutions relative to the parent polypeptide of SEQ ID NO: 3. In some embodiments, the variant polypeptide comprises the substitution I61R and one, two, three, or four additional substitutions relative to the parent polypeptide of SEQ ID NO: 3. In some embodiments, the variant polypeptide comprises the substitution G223R and one, two, three, or four additional substitutions relative to the parent polypeptide of SEQ ID NO: 3. In some embodiments, the variant polypeptide comprises the substitution N109R and one, two, three, or four additional substitutions relative to the parent polypeptide of SEQ ID NO: 3. In some embodiments, the variant polypeptide comprises the substitution D719R and one, two, three, or four additional substitutions relative to the parent polypeptide of SEQ ID NO: 3. In some embodiments, the variant polypeptide comprises the amino acid substitutions relative to the parent polypeptide of SEQ ID NO: 3 as shown in Table 3.

TABLE 3
Variants of SEQ ID NO: 3 with Multiple
Amino Acid Substitutions.

| # of Substitutions | Amino Acid Substitutions |
|---|---|
| 2 | P14R, E311R |
| 2 | P14R, D32R |
| 2 | P14R, I61R |
| 2 | P14R, G223R |
| 2 | E311R, D32R |
| 2 | E311R, I61R |
| 2 | E311R, G223R |
| 2 | D32R, I61R |
| 2 | D32R, G223R |
| 2 | I61R, G223R |
| 3 | P14R, E311R, D32R |
| 3 | P14R, E311R, I61R |
| 3 | P14R, E311R, G223R |
| 3 | P14R, D32R, I61R |
| 3 | P14R, D32R, G223R |
| 3 | P14R, I61R, G223R |
| 3 | E311R, D32R, I61R |
| 3 | E311R, D32R, G223R |
| 3 | E311R, I61R, G223R |
| 3 | D32R, I61R, G223R |
| 4 | P14R, E311R, D32R, I61R |
| 4 | P14R, E311R, D32R, G223R |
| 4 | P14R, E311R, I61R, G223R |
| 4 | P14R, D32R, I61R, G223R |
| 4 | E311R, D32R, I61R, G223R |
| 5 | P14R, E311R, D32R, I61R, G223R |
| 2 | D32R, N109R |
| 2 | N109R, G223R |
| 2 | N109R, E311R |
| 3 | D32R, N109R, G223R |
| 3 | D32R, N109R, E311R |
| 3 | N109R, G223R, E311R |
| 2 | D32R, D719R |
| 2 | N109R, D719R |
| 2 | G223R, D719R |
| 2 | E311R, D719R |
| 3 | D32R, N109R, D719R |
| 3 | D32R, G223R, D719R |
| 3 | D32R, E311R, D719R |
| 3 | N109R, G223R, D719R |
| 3 | N109R, E311R, D719R |
| 3 | G223R, E311R, D719R |
| 4 | D32R, N109R, G223R, E311R |
| 4 | D32R, N109R, G223R, D719R |
| 4 | D32R, N109R, E311R, D719R |
| 4 | D32R, G223R, E311R, D719R |
| 4 | N109R, G223R, E311R, D719R |
| 5 | D32R, N109R, G223R, E311R, D719R |

In some embodiments, the variant polypeptide comprises an amino acid having at least 95%, 96%, 97%, 98%, or 99% identity to SEQ ID NO: 3 and comprising a substitution at position P14 (e.g., a P14R substitution), E311 (e.g., an E311R), and D32 (e.g., a D32R substitution) relative to SEQ ID NO: 3 (e.g., a P14R, E311R, D32R variant polypeptide).

In some embodiments, the variant polypeptide comprises an amino acid having at least 95%, 96%, 97%, 98%, or 99% identity to SEQ ID NO: 3 and comprising a substitution at position P14 (e.g., a P14R substitution), E311 (e.g., an E311R), and G223 (e.g., a G223R substitution) relative to SEQ ID NO: 3 (e.g., a P14R, E311R, G223R variant polypeptide).

In some embodiments, the variant polypeptide comprises an amino acid having at least 95%, 96%, 97%, 98%, or 99% identity to SEQ ID NO: 3 and comprising a substitution at position P14 (e.g., a P14R substitution), E311 (e.g., an E311R), D32 (e.g., a D32R substitution), and I61 (e.g., an I61R substitution) relative to SEQ ID NO: 3 (e.g., a P14R, E311R, D32R and I61R variant polypeptide).

In some embodiments, the variant polypeptide comprises an amino acid having at least 95%, 96%, 97%, 98%, or 99% identity to SEQ ID NO: 3 and comprising a substitution at position D32R (e.g., a D32R substitution), N109 (e.g., an N109R), E311 (e.g., an E311R substitution), and D719 (e.g., a D719R substitution) relative to SEQ ID NO: 3 (e.g., a D32R, N109R, E311R and D719R variant polypeptide).

In some embodiments. In some embodiments, variant polypeptide comprises an amino acid sequence having at least 95%, 96%, 97%, 98%, or 99% identity to SEQ ID NO: 3 and comprising a substitution at position K208 (e.g., a K208G substitution) relative to SEQ ID NO: 3. In some aspects, the present disclosure provides a polypeptide comprising an amino acid sequence having one or more sequence alterations (e.g., substitutions, insertions, or deletions, or any combination thereof) at up to 1, 2, 3, 4, 5, 10, 15, 20, 25, 30, or 35 amino acid positions of SEQ ID NO: 3, wherein one of the sequence alterations comprises a substitution at position K208 (e.g., a K208G substitution) relative to SEQ ID NO: 3.

In some embodiments, variant polypeptide comprises an amino acid sequence having at least 95%, 96%, 97%, 98%, or 99% identity to SEQ ID NO: 3 and comprising a substitution at position D302 (e.g., a D302G substitution) relative to SEQ ID NO: 3. In some aspects, the present disclosure provides a polypeptide comprising an amino acid sequence having one or more sequence alterations (e.g., substitutions, insertions, or deletions, or any combination thereof) at up to 1, 2, 3, 4, 5, 10, 15, 20, 25, 30, or 35 amino acid positions of SEQ ID NO: 3, wherein one of the sequence alterations comprises a substitution at position D302 (e.g., a D302G substitution) relative to SEQ ID NO: 3.

In some embodiments, variant polypeptide comprises an amino acid sequence having at least 95%, 96%, 97%, 98%, or 99% identity to SEQ ID NO: 3 and comprising a substitution at position D590 (e.g., a D590G substitution) relative to SEQ ID NO: 3. In some aspects, the present disclosure provides a polypeptide comprising an amino acid sequence having one or more sequence alterations (e.g., substitutions, insertions, or deletions, or any combination thereof) at up to 1, 2, 3, 4, 5, 10, 15, 20, 25, 30, or 35 amino acid positions of SEQ ID NO: 3, wherein one of the sequence alterations comprises a substitution at position D590 (e.g., a D590G substitution) relative to SEQ ID NO: 3.

In some embodiments, variant polypeptide comprises an amino acid sequence having at least 95%, 96%, 97%, 98%, or 99% identity to SEQ ID NO: 3 and comprising a substitution at position E154 (e.g., an E154G substitution) relative to SEQ ID NO: 3. In some aspects, the present disclosure provides a polypeptide comprising an amino acid sequence having one or more sequence alterations (e.g., substitutions, insertions, or deletions, or any combination thereof) at up to 1, 2, 3, 4, 5, 10, 15, 20, 25, 30, or 35 amino acid positions of SEQ ID NO: 3, wherein one of the sequence alterations comprises a substitution at position E154 (e.g., an E154G substitution) relative to SEQ ID NO: 3.

In some embodiments, variant polypeptide comprises an amino acid sequence having at least 95%, 96%, 97%, 98%, or 99% identity to SEQ ID NO: 3 and comprising a substitution at position D567 (e.g., a D567G substitution) relative to SEQ ID NO: 3. In some aspects, the present disclosure provides a polypeptide comprising an amino acid sequence having one or more sequence alterations (e.g., substitutions, insertions, or deletions, or any combination thereof) at up to 1, 2, 3, 4, 5, 10, 15, 20, 25, 30, or 35 amino acid positions of SEQ ID NO: 3, wherein one of the sequence alterations comprises a substitution at position D567 (e.g., a D567G substitution) relative to SEQ ID NO: 3.

In some embodiments, variant polypeptide comprises an amino acid sequence having at least 95%, 96%, 97%, 98%, or 99% identity to SEQ ID NO: 3 and comprising a substitution at position L38 (e.g., an L38G substitution) relative to SEQ ID NO: 3. In some aspects, the present disclosure provides a polypeptide comprising an amino acid sequence having one or more sequence alterations (e.g., substitutions, insertions, or deletions, or any combination thereof) at up to 1, 2, 3, 4, 5, 10, 15, 20, 25, 30, or 35 amino acid positions of SEQ ID NO: 3, wherein one of the sequence alterations comprises a substitution at position L38 (e.g., an L38G substitution) relative to SEQ ID NO: 3.

In some embodiments, variant polypeptide comprises an amino acid sequence having at least 95%, 96%, 97%, 98%, or 99% identity to SEQ ID NO: 3 and comprising a substitution at position D145 (e.g., a D145G substitution) relative to SEQ ID NO: 3. In some aspects, the present disclosure provides a polypeptide comprising an amino acid sequence having one or more sequence alterations (e.g., substitutions, insertions, or deletions, or any combination thereof) at up to 1, 2, 3, 4, 5, 10, 15, 20, 25, 30, or 35 amino acid positions of SEQ ID NO: 3, wherein one of the sequence alterations comprises a substitution at position D145 (e.g., a D145G substitution) relative to SEQ ID NO: 3.

In some embodiments, variant polypeptide comprises an amino acid sequence having at least 95%, 96%, 97%, 98%, or 99% identity to SEQ ID NO: 3 and comprising a substitution at position C13 (e.g., a C13G substitution) relative to SEQ ID NO: 3. In some aspects, the present disclosure provides a polypeptide comprising an amino acid sequence having one or more sequence alterations (e.g., substitutions, insertions, or deletions, or any combination thereof) at up to 1, 2, 3, 4, 5, 10, 15, 20, 25, 30, or 35 amino acid positions of SEQ ID NO: 3, wherein one of the sequence alterations comprises a substitution at position C13 (e.g., a C13G substitution) relative to SEQ ID NO: 3.

In some embodiments, variant polypeptide comprises an amino acid sequence having at least 95%, 96%, 97%, 98%, or 99% identity to SEQ ID NO: 3 and comprising a substitution at position T338 (e.g., a T338G substitution) relative to SEQ ID NO: 3. In some aspects, the present disclosure provides a polypeptide comprising an amino acid sequence having one or more sequence alterations (e.g., substitutions, insertions, or deletions, or any combination thereof) at up to 1, 2, 3, 4, 5, 10, 15, 20, 25, 30, or 35 amino acid positions of SEQ ID NO: 3, wherein one of the sequence alterations comprises a substitution at position T338 (e.g., a T338G substitution) relative to SEQ ID NO: 3.

In some embodiments, variant polypeptide comprises an amino acid sequence having at least 95%, 96%, 97%, 98%, or 99% identity to SEQ ID NO: 3 and comprising a substitution at position P14 (e.g., a P14G substitution) relative to SEQ ID NO: 3. In some aspects, the present disclosure provides a polypeptide comprising an amino acid sequence having one or more sequence alterations (e.g., substitutions, insertions, or deletions, or any combination thereof) at up to 1, 2, 3, 4, 5, 10, 15, 20, 25, 30, or 35 amino acid positions of SEQ ID NO: 3, wherein one of the sequence alterations comprises a substitution at position P14 (e.g., a P14G substitution) relative to SEQ ID NO: 3.

In some embodiments, variant polypeptide comprises an amino acid sequence having at least 95%, 96%, 97%, 98%, or 99% identity to SEQ ID NO: 3 and comprising a substitution at position D55 (e.g., a D55G substitution) relative to SEQ ID NO: 3. In some aspects, the present disclosure provides a polypeptide comprising an amino acid sequence having one or more sequence alterations (e.g., substitutions, insertions, or deletions, or any combination thereof) at up to 1, 2, 3, 4, 5, 10, 15, 20, 25, 30, or 35 amino acid positions of SEQ ID NO: 3, wherein one of the sequence alterations comprises a substitution at position D55 (e.g., a D55G substitution) relative to SEQ ID NO: 3.

In some embodiments, variant polypeptide comprises an amino acid sequence having at least 95%, 96%, 97%, 98%, or 99% identity to SEQ ID NO: 3 and comprising a substitution at position K221 (e.g., a K221G substitution) relative to SEQ ID NO: 3. In some aspects, the present disclosure provides a polypeptide comprising an amino acid sequence having one or more sequence alterations (e.g., substitutions, insertions, or deletions, or any combination thereof) at up to 1, 2, 3, 4, 5, 10, 15, 20, 25, 30, or 35 amino acid positions of SEQ ID NO: 3, wherein one of the sequence alterations comprises a substitution at position K221 (e.g., a K221G substitution) relative to SEQ ID NO: 3.

In some embodiments, variant polypeptide comprises an amino acid sequence having at least 95%, 96%, 97%, 98%, or 99% identity to SEQ ID NO: 3 and comprising a substitution at position K35 (e.g., a 20) K35G substitution) relative to SEQ ID NO: 3. In some aspects, the present disclosure provides a polypeptide comprising an amino acid sequence having one or more sequence alterations (e.g., substitutions, insertions, or deletions, or any combination thereof) at up to 1, 2, 3, 4, 5, 10, 15, 20, 25, 30, or 35 amino acid positions of SEQ ID NO: 3, wherein one of the sequence alterations comprises a substitution at position K35 (e.g., a K35G substitution) relative to SEQ ID NO: 3.

25 In some embodiments, variant polypeptide comprises an amino acid sequence having at least 95%, 96%, 97%, 98%, or 99% identity to SEQ ID NO: 3 and comprising a substitution at position E736 (e.g., an E736G substitution) relative to SEQ ID NO: 3. In some aspects, the present disclosure provides a polypeptide comprising an amino acid sequence having one or more sequence alterations (e.g., substitutions, insertions, or deletions, or any combination thereof) at up to 1, 2, 3, 4, 5, 10, 15, 20, 25, 30, or 35 amino acid positions of SEQ ID NO: 3, wherein one of the sequence alterations comprises a substitution at position E736 (e.g., an E736G substitution) relative to SEQ ID NO: 3.

In some embodiments, the variant polypeptide comprises an amino acid sequence having at least 95%, 96%, 97%, 98%, or 99% identity to SEQ ID NO: 3 and comprises a substitution at position K208 (e.g., a K208G substitution), D302 (e.g., a D302G substitution), D590 (e.g., a D590G substitution), E154 (e.g., an E154G substitution), D567 (e.g., a D567G substitution), L38 (e.g., an L38G substitution), D145 (e.g., a D145G substitution), C13 (e.g., a C13G substitution), T338 (e.g., a T338G substitution), P14 (e.g., a P14G substitution), D55 (e.g., a D55G substitution), K221 (e.g., a K221G substitution), K35 (e.g., a K35G substitution), E736 (e.g., an E736G substitution), or any combination thereof, relative to SEQ ID NO: 3 (e.g., a variant polypeptide comprising 2, 3, 4, 5, 6, 7, 8, 9, 10, 11, or all of K208G, D302G, D590G, E154G, D567G, L38G, D145G, C13G, T338G, P14G, D55G, K221G, K35G, E736G).

In some embodiments, the variant polypeptide comprises an amino acid sequence having at least 95%, 96%, 97%, 98%, or 99% identity to SEQ ID NO: 3 and comprises:
i) a substitution selected from one or more of (e.g., 1, 2, 3, 4, 5, 6, or all of) a substitution at position P14 (e.g., a P14R substitution), E311 (e.g., an E311R substitution), D32 (e.g., a D32R substitution), 161 (e.g., an I61R substitution), G223 (e.g., a G223R substitution), N109 (e.g., an N109R substitution), and D719 (e.g., a D719R substitution); and
ii) a substitution selected from one or more of (e.g., 1, 2, 3, 4, 5, 6, 7, 8, 9, 10, 11, 12, or all of) a substitution at position K208 (e.g., a K208G substitution), D302 (e.g., a D302G substitution), D590 (e.g., a D590G substitution), E154 (e.g., an E154G substitution), D567 (e.g., a D567G substitution), L38 (e.g., an L38G substitution), D145 (e.g., a D145G substitution), C13 (e.g., a C13G substitution), T338 (e.g., a T338G substitution), P14 (e.g., a P14G substitution), D55 (e.g., a D55G substitution), K221 (e.g., a K221G substitution), K35 (e.g., a K35G substitution), and E736 (e.g., an E736G substitution),

In some embodiments, the variant polypeptide comprises and amino acid sequence having at least 95%, 96%, 97%, 98%, or 99% identity to SEQ ID NO: 3 and comprises a substitution at P14 (e.g., a P14R substitution) and a substitution selected from one or more of (e.g., 1, 2, 3, 4, 5, 6, 7, 8, 9, 10, 11, or all of) a substitution at position K208 (e.g., a K208G substitution), D302 (e.g., a D302G substitution), D590 (e.g., a D590G substitution), E154 (e.g., an E154G substitution), D567 (e.g., a D567G substitution), L38 (e.g., an L38G substitution), D145 (e.g., a D145G substitution), C13 (e.g., a C13G substitution), T338 (e.g., a T338G substitution), D55 (e.g., a D55G substitution), K221 (e.g., a K221G substitution), K35 (e.g., a K35G substitution), and E736 (e.g., an E736G substitution), For instance, in some embodiments, the variant polypeptide comprises a P14R substitution and one or more of: a K208G substitution, a D302G substitution, a D590G substitution, an E154G substitution, a D567G substitution, a L38G substitution, a D145G substitution, a C13G substitution, a T338G substitution, a P14G substitution, a D55G substitution, a K221G substitution, a K35G substitution, and an E736G substitution.

In some embodiments, the variant polypeptide comprises and amino acid sequence having at least 95%, 96%, 97%, 98%, or 99% identity to SEQ ID NO: 3 and comprises a substitution at E311 (e.g., an E311R substitution) and a substitution selected from one or more of (e.g., 1, 2, 3, 4, 5, 6, 7, 8, 9, 10, 11, 12, or all of) a substitution at position K208 (e.g., a K208G substitution), D302 (e.g., a D302G substitution), D590 (e.g., a D590G substitution), E154 (e.g., an E154G substitution), D567 (e.g., a D567G substitution), L38 (e.g., an L38G substitution), D145 (e.g., a D145G substitution), C13 (e.g., a C13G substitution), T338 (e.g., a T338G substitution), P14 (e.g., a P14G substitution), D55 (e.g., a D55G substitution), K221 (e.g., a K221G substitution), K35 (e.g., a K35G substitution), and E736 (e.g., an E736G substitution), For instance, in some embodiments, the variant polypeptide comprises an E311R substitution and one or more of: a K208G substitution, a D302G substitution, a D590G substitution, an E154G substitution, a D567G substitution, a L38G substitution, a D145G substitution, a C13G substitution, a T338G substitution, a P14G substitution, a D55G substitution, a K221G substitution, a K35G substitution, and an E736G substitution.

In some embodiments, the variant polypeptide comprises and amino acid sequence having at least 95%, 96%, 97%, 98%, or 99% identity to SEQ ID NO: 3 and comprises a substitution at D32 (e.g., a D32R substitution) and a substitution selected from one or more of (e.g., 1, 2, 3, 4, 5, 6, 7, 8, 9, 10, 11, 12, or all of) a substitution at position K208 (e.g., a K208G substitution), D302 (e.g., a D302G substitution), D590 (e.g., a D590G substitution), E154 (e.g., an E154G substitution), D567 (e.g., a D567G substitution), L38 (e.g., an L38G substitution), D145 (e.g., a D145G substitution), C13 (e.g., a C13G substitution), T338 (e.g., a T338G substitution), P14 (e.g., a P14G substitution), D55 (e.g., a D55G substitution), K221 (e.g., a K221G substitution), K35 (e.g., a K35G substitution), and E736 (e.g., an E736G substitution), For instance, in some embodiments, the variant polypeptide comprises a D32R substitution and one or more of: a K208G substitution, a D302G substitution, a D590G substitution, an E154G substitution, a D567G substitution, a L38G substitution, a D145G substitution, a C13G substitution, a T338G substitution, a P14G substitution, a D55G substitution, a K221G substitution, a K35G substitution, and an E736G substitution, 20) In some embodiments, the variant polypeptide comprises and amino acid sequence having at least 95%, 96%, 97%, 98%, or 99% identity to SEQ ID NO: 3 and comprises a substitution at position 161 (e.g., an 161R substitution) and a substitution selected from one or more of (e.g., 1, 2, 3, 4, 5, 6, 7, 8, 9, 10, 11, 12, or all of) a substitution at position K208 (e.g., a K208G substitution), D302 (e.g., a D302G substitution), D590 (e.g., a D590G substitution), E154 (e.g., an E154G substitution), D567 (e.g., a D567G substitution), L38 (e.g., an L38G substitution), D145 (e.g., a D145G substitution), C13 (e.g., a C13G substitution), T338 (e.g., a T338G substitution), P14 (e.g., a P14G substitution), D55 (e.g., a D55G substitution), K221 (e.g., a K221G substitution), K35 (e.g., a K35G substitution), and E736 (e.g., an E736G substitution), For instance, in some embodiments, the variant polypeptide comprises an 161R substitution and one or more of: a K208G substitution, a D302G substitution, a D590G substitution, an E154G substitution, a D567G substitution, a L38G substitution, a D145G substitution, a C13G substitution, a T338G substitution, a P14G substitution, a D55G substitution, a K221G substitution, a K35G substitution, and an E736G substitution.

In some embodiments, the variant polypeptide comprises and amino acid sequence having at least 95%, 96%, 97%, 98%, or 99% identity to SEQ ID NO: 3 and comprises a substitution at position G223 (e.g., a G223R substitution) and a substitution selected from one or more of (e.g., 1, 2, 3, 4, 5, 6, 7, 8, 9, 10, 11, 12, or all of) a substitution at position K208 (e.g., a K208G substitution), D302 (e.g., a D302G substitution), D590 (e.g., a D590G substitution), E154 (e.g., an E154G substitution), D567 (e.g., a D567G substitution), L38 (e.g., an L38G substitution), D145 (e.g., a D145G substitution), C13 (e.g., a C13G substitution), T338 (e.g., a T338G substitution), P14 (e.g., a P14G substitution), D55 (e.g., a D55G substitution), K221 (e.g., a K221G substitution), K35 (e.g., a K35G substitution), and E736 (e.g., an E736G substitution), For instance, in some embodiments, the variant polypeptide comprises a G223R substitution and one or more of: a K208G substitution, a D302G substitution, a D590G substitution, an E154G substitution, a D567G substitution, a L38G substitution, a D145G substitution, a C13G substitution, a T338G substitution, a P14G substitution, a D55G substitution, a K221G substitution, a K35G substitution, and an E736G substitution.

In some embodiments, the variant polypeptide comprises and amino acid sequence having at least 95%, 96%, 97%, 98%, or 99% identity to SEQ ID NO: 3 and comprises a substitution at position N109 (e.g., an N109R substitution) and a substitution selected from one or more of (e.g., 1, 2, 3, 4, 5, 6, 7, 8, 9, 10, 11, 12, or all of) a substitution at position K208 (e.g., a K208G substitution), D302 (e.g., a D302G substitution), D590 (e.g., a D590G substitution), E154 (e.g., an E154G substitution), D567 (e.g., a D567G substitution), L38 (e.g., an L38G substitution), D145 (e.g., a D145G substitution), C13 (e.g., a C13G substitution), T338 (e.g., a T338G substitution), P14 (e.g., a P14G substitution), D55 (e.g., a D55G substitution), K221 (e.g., a K221G substitution), K35 (e.g., a K35G substitution), and E736 (e.g., an E736G substitution), For instance, in some embodiments, the variant polypeptide comprises an N109R substitution and one or more of: a K208G substitution, a D302G substitution, a D590G substitution, an E154G substitution, a D567G substitution, a L38G substitution, a D145G substitution, a C13G substitution, a T338G substitution, a P14G substitution, a D55G substitution, a K221G substitution, a K35G substitution, and an E736G substitution.

In some embodiments, the variant polypeptide comprises and amino acid sequence having at least 95%, 96%, 97%, 98%, or 99% identity to SEQ ID NO: 3 and comprises a substitution at position D719 (e.g., a D719R substitution) and a substitution selected from one or more of (e.g., 1, 2, 3, 4, 5, 6, 7, 8, 9, 10, 11, 12, or all of) a substitution at position K208 (e.g., a K208G substitution), D302 (e.g., a D302G substitution), D590 (e.g., a D590G substitution), E154 (e.g., an E154G substitution), D567 (e.g., a D567G substitution), L38 (e.g., an L38G substitution), D145 (e.g., a D145G substitution), C13 (e.g., a C13G substitution), T338 (e.g., a T338G substitution), P14 (e.g., a P14G substitution), D55 (e.g., a D55G substitution), K221 (e.g., a K221G substitution), K35 (e.g., a K35G substitution), and E736 (e.g., an E736G substitution), For instance, in some embodiments, the variant polypeptide comprises a D719R substitution and one or more of: a K208G substitution, a D302G substitution, a D590G substitution, an E154G substitution, a D567G substitution, a L38G substitution, a D145G substitution, a C13G substitution, a T338G substitution, a P14G substitution, a D55G substitution, a K221G substitution, a K35G substitution, and an E736G substitution.

In some embodiments, the variant polypeptide comprises and amino acid sequence having at least 95%, 96%, 97%, 98%, or 99% identity to SEQ ID NO: 3 and comprises a substitution at position P14 (e.g., a P14R substitution), a substitution at position E311 (e.g., an E311R substitution), a substitution at position D32 (e.g., a D32R substitution) and a substitution selected from one or more of (e.g., 1, 2, 3, 4, 5, 6, 7, 8, 9, 10, 11, or all of) a substitution at position K208 (e.g., a K208G substitution), D302 (e.g., a D302G substitution), D590 (e.g., a D590G substitution), E154 (e.g., an E154G substitution), D567 (e.g., a D567G substitution), L38 (e.g., an L38G substitution), D145 (e.g., a D145G substitution), C13 (e.g., a C13G substitution), T338 (e.g., a T338G substitution), P14 (e.g., a P14G substitution), D55 (e.g., a D55G substitution), K221 (e.g., a K221G substitution), K35 (e.g., a K35G substitution), and E736 (e.g., an E736G substitution), For instance, in some embodiments, the variant polypeptide comprises a P14R substitution, an E311R substitution, a D32R substitution, and a substitution selected from one or more of (e.g., 1, 2, 3, 4, 5, 6, 7, 8, 9, 10, 11, or all of) a K208G substitution, a D302G substitution, a D590G substitution, an E154G substitution, a D567G substitution, a L38G substitution, a D145G substitution, a C13G substitution, a T338G substitution, a D55G substitution, a K221G substitution, a K35G substitution, and an E736G substitution.

In some embodiments, the variant polypeptide comprises and amino acid sequence having at least 95%, 96%, 97%, 98%, or 99% identity to SEQ ID NO: 3 and comprises a substitution at position P14 (e.g., a P14R substitution), a substitution at position E311 (e.g., an E311R substitution), a substitution at position G223 (e.g., a G223R substitution) and a substitution selected from one or more of (e.g., 1, 2, 3, 4, 5, 6, 7, 8, 9, 10, 11, 12, or all of) a substitution at position K208 (e.g., a K208G substitution), D302 (e.g., a D302G substitution), D590 (e.g., a D590G substitution), E154 (e.g., an E154G substitution), D567 (e.g., a D567G substitution), L38 (e.g., an L38G substitution), D145 (e.g., a D145G substitution), C13 (e.g., a C13G substitution), T338 (e.g., a T338G substitution), P14 (e.g., a P14G substitution), D55 (e.g., a D55G substitution), K221 (e.g., a K221G substitution), K35 (e.g., a K35G substitution), and E736 (e.g., an E736G substitution), For instance, in some embodiments, the variant polypeptide comprises a P14R substitution, an E311R substitution, a G223R substitution, and a substitution selected from one or more of (e.g., 1, 2, 3, 4, 5, 6, 7, 8, 9, 10, 11, 12, or all of) a K208G substitution, a D302G substitution, a D590G substitution, an E154G substitution, a D567G substitution, a L38G substitution, a D145G substitution, a C13G substitution, a T338G substitution, a D55G substitution, a K221G substitution, a K35G substitution, and an E736G substitution.

In some embodiments, the variant polypeptide comprises and amino acid sequence having at least 95%, 96%, 97%, 98%, or 99% identity to SEQ ID NO: 3 and comprises a substitution at position P14 (e.g., a P14R substitution), a substitution at position E311 (e.g., an E311R substitution), a substitution at position D32 (e.g., a D32R substitution), a substitution at 161 (e.g., an 161R substitution), and a substitution selected from one or more of (e.g., 1, 2, 3, 4, 5, 6, 7, 8, 9, 10, 11, 12, or all of) a substitution at position K208 (e.g., a K208G substitution), D302 (e.g., a D302G substitution), D590 (e.g., a D590G substitution), E154 (e.g., an E154G substitution), D567 (e.g., a D567G substitution), L38 (e.g., an L38G substitution), D145 (e.g., a D145G substitution), C13 (e.g., a C13G substitution), T338 (e.g., a T338G substitution), P14 (e.g., a P14G substitution), D55 (e.g., a D55G substitution), K221 (e.g., a K221G substitution), K35 (e.g., a K35G 10) substitution), and E736 (e.g., an E736G substitution), For instance, in some embodiments, the variant polypeptide comprises a P14R substitution, an E31IR substitution, a D32R substitution, an 16IR substitution, and a substitution selected from one or more of (e.g., 1, 2, 3, 4, 5, 6, 7, 8, 9, 10, 11, 12, or all of) a K208G substitution, a D302G substitution, a D590G substitution, an E154G substitution, a D567G substitution, a L38G substitution, a D145G substitution, a C13G substitution, a T338G substitution, a D55G substitution, a K221G substitution, a K35G substitution, and an E736G substitution, 15 In some embodiments, the variant polypeptide comprises and amino acid sequence having at least 95%, 96%, 97%, 98%, or 99% identity to SEQ ID NO: 3 and comprises a substitution at position D32 (e.g., a D32R substitution), a substitution at position N109 (e.g., an N109R substitution), a substitution at position E311 (e.g., an E311R substitution), a substitution at D719 (e.g., a D719R substitution), and a substitution selected from one or more of (e.g., 1, 2, 3, 4, 5, 6, 7, 8, 9, 10, 11, 12, or all of) a substitution at position 20) K208 (e.g., a K208G substitution), D302 (e.g., a D302G substitution), D590 (e.g., a D590G substitution), E154 (e.g., an E154G substitution), D567 (e.g., a D567G substitution), L38 (e.g., an L38G substitution), D145 (e.g., a D145G substitution), C13 (e.g., a C13G substitution), T338 (e.g., a T338G substitution), P14 (e.g., a P14G substitution), D55 (e.g., a D55G substitution), K221 (e.g., a K221G substitution), K35 (e.g., a K35G substitution), and E736 (e.g., an E736G substitution), For instance, in some embodiments, the variant polypeptide comprises a D32R substitution, an N109R substitution, an E311R substitution, a D719R substitution, and a substitution selected from one or more of (e.g., 1, 2, 3, 4, 5, 6, 7, 8, 9, 10, 11, 12, or all of) a K208G substitution, a D302G substitution, a D590G substitution, an E154G substitution, a D567G substitution, a L38G substitution, a D145G substitution, a C13G substitution, a T338G substitution, a D55G substitution, a K221G substitution, a K35G substitution, and an E736G substitution.

In some embodiments, the variant polypeptide comprises and amino acid sequence having at least 95%, 96%, 97%, 98%, or 99% identity to SEQ ID NO: 3 and comprises a substitution at K208 (e.g., a K208G substitution) and a substitution selected from one or more of (e.g., 1, 2, 3, 4, 5, 6, 7, 8, 9, 10, 11, 12, or all of) a substitution at position P14 (e.g., a P14R substitution), E311 (e.g., an E311R substitution), D32 (e.g., a D32R substitution), 161 (e.g., an 161R substitution), G223 (e.g., a G223R substitution), N109 (e.g., an N109R substitution), and D719 (e.g., a D719R substitution), For instance, in some embodiments, the variant polypeptide comprises a K208G substitution and one or more of: a P14R substitution, an E311R substitution, a D32R substitution, an I61R substitution, a G223R substitution, an N109R substitution, and a D719R substitution.

In some embodiments, the variant polypeptide comprises and amino acid sequence having at least 95%, 96%, 97%, 98%, or 99% identity to SEQ ID NO: 3 and comprises a substitution at D302 (e.g., a D302G substitution) and a substitution selected from one or more of (e.g., 1, 2, 3, 4, 5, 6, 7, 8, 9, 10, 11, 12, or all of) a substitution at position P14 (e.g., a P14R substitution), E311 (e.g., an E311R substitution), D32 (e.g., a D32R substitution), 161 (e.g., an I61R substitution), G223 (e.g., a G223R substitution), N109 (e.g., an N109R substitution), and D719 (e.g., a D719R substitution), For instance, in some embodiments, the variant polypeptide comprises a K208G substitution and one or more of: a P14R substitution, an E311R substitution, a D32R substitution, an I61R substitution, a G223R substitution, an N109R substitution, and a D719R substitution.

In some embodiments, the variant polypeptide comprises and amino acid sequence having at least 95%, 96%, 97%, 98%, or 99% identity to SEQ ID NO: 3 and comprises a substitution at D590 (e.g., a D590G substitution) and a substitution selected from one or more of (e.g., 1, 2, 3, 4, 5, 6, 7, 8, 9, 10, 11, 12, or all of) a substitution at position P14 (e.g., a P14R substitution), E311 (e.g., an E311R substitution), D32 (e.g., a D32R substitution), 161 (e.g., an I61R substitution), G223 (e.g., a G223R substitution), N109 (e.g., an N109R substitution), and D719 (e.g., a D719R substitution), For instance, in some embodiments, the variant polypeptide comprises a K208G substitution and one or more of: a P14R substitution, an E311R substitution, a D32R substitution, an 161R substitution, a G223R substitution, an N109R substitution, and a D719R substitution.

In some embodiments, the variant polypeptide comprises and amino acid sequence having at least 95%, 96%, 97%, 98%, or 99% identity to SEQ ID NO: 3 and comprises a substitution at E154 (e.g., an E154G substitution) and a substitution selected from one or more of (e.g., 1, 2, 3, 4, 5, 6, 7, 8, 9, 10, 11, 12, or all of) a substitution at position P14 (e.g., a P14R substitution), E311 (e.g., an E311R substitution), D32 (e.g., a D32R substitution), 161 (e.g., an 161R substitution), G223 (e.g., a G223R substitution), N109 (e.g., an N109R substitution), and D719 (e.g., a D719R substitution), For instance, in some embodiments, the variant polypeptide comprises a K208G substitution and one or more of: a P14R substitution, an E311R substitution, a D32R substitution, an 161R substitution, a G223R substitution, an N109R substitution, and a D719R substitution.

In some embodiments, the variant polypeptide comprises and amino acid sequence having at least 95%, 96%, 97%, 98%, or 99% identity to SEQ ID NO: 3 and comprises a substitution at D567 (e.g., a D567G substitution) and a substitution selected from one or more of (e.g., 1, 2, 3, 4, 5, 6, 7, 8, 9, 10, 11, 12, or all of) a substitution at position P14 (e.g., a P14R substitution), E311 (e.g., an E311R substitution), D32 (e.g., a D32R substitution), 161 (e.g., an 161R substitution), G223 (e.g., a G223R substitution), N109 (e.g., an N109R substitution), and D719 (e.g., a D719R substitution), For instance, in some embodiments, the variant polypeptide comprises a K208G substitution and one or more of: a P14R substitution, an E311R substitution, a D32R substitution, an 161R substitution, a G223R substitution, an N109R substitution, and a D719R substitution.

In some embodiments, the variant polypeptide comprises and amino acid sequence having at least 95%, 96%, 97%, 98%, or 99% identity to SEQ ID NO: 3 and comprises a substitution at L38 (e.g., an L38G substitution) and a substitution selected from one or more of (e.g., 1, 2, 3, 4, 5, 6, 7, 8, 9, 10, 11, 12, or all of) a substitution at position P14 (e.g., a P14R substitution), E311 (e.g., an E311R substitution), D32 (e.g., a D32R substitution), 161 (e.g., an 161R substitution), G223 (e.g., a G223R substitution), N109 (e.g., an N109R substitution), and D719 (e.g., a D719R substitution), For instance, in some embodiments, the variant polypeptide comprises a K208G substitution and one or more of: a P14R substitution, an E311R substitution, a D32R substitution, an 161R substitution, a G223R substitution, an N109R substitution, and a D719R substitution.

In some embodiments, the variant polypeptide comprises and amino acid sequence having at least 95%, 96%, 97%, 98%, or 99% identity to SEQ ID NO: 3 and comprises a substitution at D145 (e.g., a D145G substitution) and a substitution selected from one or more of (e.g., 1, 2, 3, 4, 5, 6, 7, 8, 9, 10, 11, 12, or all of) a substitution at position P14 (e.g., a P14R substitution), E311 (e.g., an E311R substitution), D32 (e.g., a D32R substitution), 161 (e.g., an 161R substitution), G223 (e.g., a G223R substitution), N109 (e.g., an N109R substitution), and D719 (e.g., a D719R substitution), For instance, in some embodiments, the variant polypeptide comprises a K208G substitution and one or more of: a P14R substitution, an E311R substitution, a D32R substitution, an I61R substitution, a G223R substitution, an N109R substitution, and a D719R substitution.

In some embodiments, the variant polypeptide comprises and amino acid sequence having at least 95%, 96%, 97%, 98%, or 99% identity to SEQ ID NO: 3 and comprises a substitution at C13 (e.g., a C13G substitution) and a substitution selected from one or more of (e.g., 1, 2, 3, 4, 5, 6, 7, 8, 9, 10, 11, 12, or all of) a substitution at position P14 (e.g., a P14R substitution), E311 (e.g., an E311R substitution), D32 (e.g., a D32R substitution), 161 (e.g., an I61R substitution), G223 (e.g., a G223R substitution), N109 (e.g., an N109R substitution), and D719 (e.g., a D719R substitution), For instance, in some embodiments, the variant polypeptide comprises a K208G substitution and one or more of: a P14R substitution, an E311R substitution, a D32R substitution, an 161R substitution, a G223R substitution, an N109R substitution, and a D719R substitution.

In some embodiments, the variant polypeptide comprises and amino acid sequence having at least 95%, 96%, 97%, 98%, or 99% identity to SEQ ID NO: 3 and comprises a substitution at T338 (e.g., a T338G substitution) and a substitution selected from one or more of (e.g., 1, 2, 3, 4, 5, 6, 7, 8, 9, 10, 11, 12, or all of) a substitution at position P14 (e.g., a P14R substitution), E311 (e.g., an E311R substitution), D32 (e.g., a D32R substitution), 161 (e.g., an 161R substitution), G223 (e.g., a G223R substitution), N109 (e.g., an N109R substitution), and D719 (e.g., a D719R substitution), For instance, in some embodiments, the variant polypeptide comprises a K208G substitution and one or more of: a P14R substitution, an E311R substitution, a D32R substitution, an 161R substitution, a G223R substitution, an N109R substitution, and a D719R substitution.

In some embodiments, the variant polypeptide comprises and amino acid sequence having at least 95%, 96%, 97%, 98%, or 99% identity to SEQ ID NO: 3 and comprises a substitution at P14 (e.g., a P14G substitution) and a substitution selected from one or more of (e.g., 1, 2, 3, 4, 5, 6, 7, 8, 9, 10, 11, or all of) a substitution at position P14 (e.g., a P14R substitution), E311 (e.g., an E311R substitution), D32 (e.g., a D32R substitution), 161 (e.g., an I61R substitution), G223 (e.g., a G223R substitution), N109 (e.g., an N109R substitution), and D719 (e.g., a D719R substitution), For instance, in some embodiments, the variant polypeptide comprises a K208G substitution and one or more of: a P14R substitution, an E311R substitution, a D32R substitution, an I61R substitution, a G223R substitution, an N109R substitution, and a D719R substitution.

In some embodiments, the variant polypeptide comprises and amino acid sequence having at least 95%, 96%, 97%, 98%, or 99% identity to SEQ ID NO: 3 and comprises a substitution at D55 (e.g., a D55G substitution) and a substitution selected from one or more of (e.g., 1, 2, 3, 4, 5, 6, 7, 8, 9, 10, 11, 12, or all of) a substitution at position P14 (e.g., a P14R substitution), E311 (e.g., an E311R substitution), D32 (e.g., a D32R substitution), 161 (e.g., an 161R substitution), G223 (e.g., a G223R substitution), N109 (e.g., an N109R substitution), and D719 (e.g., a D719R substitution), For instance, in some embodiments, the variant polypeptide comprises a K208G substitution and one or more of: a P14R substitution, an E311R substitution, a D32R substitution, an 161R substitution, a G223R substitution, an N109R substitution, and a D719R substitution.

In some embodiments, the variant polypeptide comprises and amino acid sequence having at least 95%, 96%, 97%, 98%, or 99% identity to SEQ ID NO: 3 and comprises a substitution at K221 (e.g., a K221G substitution) and a substitution selected from one or more of (e.g., 1, 2, 3, 4, 5, 6, 7, 8, 9, 10, 11, 12, or all of) a substitution at position P14 (e.g., a P14R substitution), E311 (e.g., an E311R substitution), D32 (e.g., a D32R substitution), 161 (e.g., an 161R substitution), G223 (e.g., a G223R substitution), N109 (e.g., an N109R substitution), and D719 (e.g., a D719R substitution), For instance, in some embodiments, the variant polypeptide comprises a K208G substitution and one or more of: a P14R substitution, an E311R substitution, a D32R substitution, an 161R substitution, a G223R substitution, an N109R substitution, and a D719R substitution.

In some embodiments, the variant polypeptide comprises and amino acid sequence having at least 95%, 96%, 97%, 98%, or 99% identity to SEQ ID NO: 3 and comprises a substitution at K35 (e.g., a K35G substitution) and a substitution selected from one or more of (e.g., 1, 2, 3, 4, 5, 6, 7, 8, 9, 10, 11, 12, or all of) a substitution at position P14 (e.g., a P14R substitution), E311 (e.g., an E311R substitution), D32 (e.g., a D32R substitution), 161 (e.g., an 161R substitution), G223 (e.g., a G223R substitution), N109 (e.g., an N109R substitution), and D719 (e.g., a D719R substitution), For instance, in some embodiments, the variant polypeptide comprises a K208G substitution and one or more of: a P14R substitution, an E311R substitution, a D32R substitution, an 161R substitution, a G223R substitution, an N109R substitution, and a D719R substitution.

In some embodiments, the variant polypeptide comprises and amino acid sequence having at least 95%, 96%, 97%, 98%, or 99% identity to SEQ ID NO: 3 and comprises a substitution at E736 (e.g., an E736G substitution) and a substitution selected from one or more of (e.g., 1, 2, 3, 4, 5, 6, 7, 8, 9, 10, 11, 12, or all of) a substitution at position P14 (e.g., a P14R substitution), E311 (e.g., an E311R substitution), D32 (e.g., a D32R substitution), 161 (e.g., an 161R substitution), G223 (e.g., a G223R substitution), N109 (e.g., an N109R substitution), and D719 (e.g., a D719R substitution), For instance, in some embodiments, the variant polypeptide comprises a K208G substitution and one or more of: a P14R substitution, an E31IR substitution, a D32R substitution, an 161R substitution, a G223R substitution, an N109R substitution, and a D719R substitution.

In some embodiments, the variant polypeptide comprises at least one RuvC motif or a RuvC domain.

Although the changes described herein may be one or more amino acid changes, changes to the variant polypeptide may also be of a substantive nature, such as fusion of polypeptides as amino- and/or carboxyl-terminal extensions. For example, the variant polypeptide may contain additional peptides, e.g., one or more peptides. Examples of additional peptides may include epitope peptides for labelling, such as a polyhistidine tag (His-tag), Myc, and FLAG. In some embodiments, the variant polypeptide described herein can be fused to a detectable moiety such as a fluorescent protein (e.g., green fluorescent protein (GFP) or yellow fluorescent protein (YFP)).

In some embodiments, the variant polypeptide comprises at least one (e.g., two, three, four, five, six, or more) nuclear localization signal (NLS). In some embodiments, the variant polypeptide comprises at least one (e.g., two, three, four, five, six, or more) nuclear export signal (NES). In some embodiments, the variant polypeptide comprises at least one (e.g., two, three, four, five, six, or more) NLS and at least one (e.g., two, three, four, five, six, or more) NES.

In some embodiments, the variant polypeptide described herein can be self-inactivating. See, Epstein et al., “Engineering a Self-Inactivating CRISPR System for AAV Vectors,” Mol. Ther., 24 (2016): S50, which is incorporated by reference in its entirety.

In some embodiments, the nucleotide sequence encoding the variant polypeptide described herein can be codon-optimized for use in a particular host cell or organism. For example, the nucleic acid can be codon-optimized for any non-human eukaryote including mice, rats, rabbits, dogs, livestock, or non-human primates. Codon usage tables are readily available, for example, at the “Codon Usage Database” available at www.kazusa.orjp/codon/and these tables can be adapted in a number of ways. See Nakamura et al. Nucl. Acids Res, 28:292 (2000), which is incorporated herein by reference in its entirety. Computer algorithms for codon optimizing a particular sequence for expression in a particular host cell are also available, such as Gene Forge (Aptagen: Jacobus, PA).

### Functionality of Variant Polypeptides

As used herein, a “biologically active portion” is a portion that retains at least one function (e.g., completely, partially, minimally) of the parent polypeptide (e.g., a “minimal” or “core” domain). In some embodiments, the variant polypeptide retains enzymatic activity at least as active as the parent polypeptide. Accordingly, in some embodiments, a variant polypeptide has enzymatic activity greater than the parent polypeptide.

In some embodiments, the variant polypeptide has reduced nuclease activity or is a nuclease dead polypeptide. As used herein, catalytic residues of a polypeptide disclosed herein comprise D328 and E530. In some embodiments, a variant polypeptide comprising a substitution at D328 and E530 (e.g., D328A and E530A) exhibits reduced nuclease activity or no nuclease activity relative to a parent polypeptide. In some embodiments, a variant polypeptide comprising a substitution at D684, D646, or D621 (e.g., D684A, D646A, or D621A) exhibits reduced nuclease activity or no nuclease activity relative to a parent polypeptide.

In an aspect, the invention provides methods for introducing an alteration or mutation into the parent polypeptide sequence to enhance binary complex formation, RNA guide binding activity, and/or RNA guide binding specificity.

In an aspect, the invention also provides methods for introducing an alteration or mutation into the parent polypeptide sequence to enhance ternary complex formation, on-target binding affinity, on-target binding activity, on-target binding, and/or on-target binding specificity. In an aspect, the invention also provides methods for introducing an alteration or mutation into the parent polypeptide sequence to enhance on-target binding affinity (e.g., affinity or time it takes to interact with target), on-target binding activity, on-target binding (e.g., strength of interaction with target), and/or on-target binding specificity (e.g., preference for specific target) of a binary complex (e.g., ribonucleoprotein). In some embodiments, an alteration or mutation is introduced to the parent polypeptide sequence to produce a variant polypeptide that has increased on-target binding and/or activity. Also, in such embodiments, off-target binding and/or activity can be decreased in the variant polypeptide, as compared to the parent polypeptide. Moreover, there can be increased or decreased specificity as to on-target binding vs, off-target binding. In some embodiments, an alteration or mutation is introduced to the parent polypeptide sequence to produce a variant polypeptide, that when complexed with an RNA guide, has increased on-target binding. Also, in such embodiments, off-target binding can be decreased in the complex comprising the variant polypeptide and RNA guide. Moreover, there can be increased or decreased specificity as to on-target binding/activity vs, off-target binding/activity. In certain embodiments, an alteration or mutation is introduced to the parent polypeptide sequence to produce a variant polypeptide that enhances stability and/or protein-RNA interactions. In certain embodiments, the variant polypeptide includes at least one alteration that promotes stability and/or RNA interactions as well as enzymatic activity of the variant polypeptide, as compared to a parent polypeptide.

In some embodiments, the variant polypeptide of the present invention has enzymatic activity equivalent to or greater than the parent polypeptide. In some embodiments, the variant polypeptide of the present invention has enzymatic activity at a temperature range from about 20° C., to about 90° C. In some embodiments, the variant polypeptide of the present invention has enzymatic activity at a temperature of about 20° C., to about 25° C. or at a temperature of about 37° C.

In some embodiments, the variant polypeptide comprises at least one alteration that enhances affinity to RNA (e.g., RNA affinity), as compared to a parent polypeptide. In some embodiments, the variant polypeptide exhibits enhanced RNA affinity, as compared to a parent polypeptide, at a temperature lower than about any one of 20° C., 21° C., 22° C., 23° C. 24° C. 25° C., 26° C., 27° C., 28° C., 29° C., 30° C., 31° C. 32° C., 33° C., 34° C., 35° C., 36° C., 37° C., 38° C., 39° C., 40° C. 41ºC. 42° C., 43° C., 44° C., 45° C., 50° C., 51° C., 52° C., 53° C., 54° C., 55° C., 56° C., 57° C., 58° C., 59° C., 60° C. or 65° C. In some embodiments, the variant polypeptide exhibits enhanced RNA affinity, as compared to a parent polypeptide, in a buffer having a pH in a range of about 7.3 to about 8.6 (e.g., about 7.5 to about 8.0, about 7.8 to 8.3, or about 8.0 to 8.6). In some embodiments, the variant polypeptide exhibits enhanced RNA affinity, as compared to a parent polypeptide, when the Tₘ value of the variant polypeptide is at least 1° C., 2° C. 3° C., 4° C., 5° C., 6° C., 7° C., 8° C., 9° C., 10° C. 11° C., 12ºC. 13° C., 14° C. 15° C., 16° C., 17° C., 18° C., 19° C., or 20° C. greater than the Tₘ value of a parent polypeptide. In one embodiment, the variant polypeptide exhibits enhanced RNA affinity when the Tₘ value of the variant polypeptide is at least 8° C. greater than the Tₘ value of the parent polypeptide.

In some embodiments, the variant polypeptide comprises at least one alteration that enhances complex formation with an RNA guide (e.g., binary complex formation), as compared to a parent polypeptide. In some embodiments, the variant polypeptide exhibits enhanced binary complex formation, as compared to a parent polypeptide, at a temperature lower than about any one of 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 50° C., 51° C., 52° C., 53° C., 54° C., 55° C., 56° C., 57° C., 58° C., 59° C., 60° C. or 65° C. In some embodiments, the variant polypeptide exhibits enhanced binary complex formation, as compared to a parent polypeptide, in a buffer having a pH in a range of about 7.3 to about 8.6 (e.g., about 7.5 to about 8.0, about 7.8 to 8.3, or about 8.0 to 8.6). In some embodiments, the variant polypeptide exhibits enhanced binary complex formation, as compared to a parent polypeptide, when the Tₘ value of the variant polypeptide is at least 1° C., 2° C., 3° C., 4° C., 5° C., 6° C., 7° C., 8° C., 9° C., 10° C., 11° C., 12° C., 13° C., 14° C., 15° C., 16° C., 17° C., 18° C., 19° C., or 20° C. greater than the Tₘ value of a parent polypeptide. In one embodiment, the variant polypeptide exhibits enhanced binary complex formation when the Tₘ value of the variant polypeptide is at least 8° C. greater than the Tₘ value of the parent polypeptide.

In some embodiments, the variant polypeptide comprises at least one alteration that enhances binding activity to an RNA guide, as compared to a parent polypeptide. In some embodiments, the variant polypeptide exhibits enhanced RNA guide binding activity, as compared to a parent polypeptide, at a temperature lower than about any one of 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 50° C., 51° C., 52° C., 53° C., 54° C., 55° C., 56° C., 57° C., 58° C., 59° C., 60° C. or 65° C. In some embodiments, the variant polypeptide exhibits enhanced RNA guide binding activity, as compared to a parent polypeptide, in a buffer having a pH in a range of about 7.3 to about 8.6 (e.g., about 7.5 to about 8.0, about 7.8 to 8.3, or about 8.0 to 8.6). In some embodiments, the variant polypeptide exhibits enhanced RNA guide binding activity, as compared to a parent polypeptide, when the Tₘ value of the variant polypeptide is at least 1° C., 2° C., 3° C., 4º° C., 5° C., 6° C., 7° C., 8° C., 9° C., 10° C., 11° C., 12° C., 13° C., 14° C., 15° C., 16° C., 17° C., 18° C., 19° C., or 20° C. greater than the Tₘ value of a parent polypeptide. In one embodiment, the variant polypeptide exhibits enhanced RNA guide binding activity when the Tₘ value of the variant polypeptide is at least 8° C. greater than the Tₘ value of the parent polypeptide.

In some embodiments, the variant polypeptide comprises at least one alteration that enhances binding specificity to an RNA guide, as compared to a parent polypeptide. In some embodiments, the variant polypeptide exhibits enhanced RNA guide binding specificity, as compared to a parent polypeptide, at a temperature lower than about any one of 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 50° C., 51° C., 52° C., 53° C., 54° C., 55° C., 56° C., 57° C., 58° C., 59° C., 60° C. or 65° C. In some embodiments, the variant polypeptide exhibits enhanced RNA guide binding specificity, as compared to a parent polypeptide, in a buffer having a pH in a range of about 7.3 to about 8.6 (e.g., about 7.5 to about 8.0, about 7.8 to 8.3, or about 8.0 to 8.6). In some embodiments, the variant polypeptide exhibits enhanced RNA guide binding specificity, as compared to a parent polypeptide, when the Tₘ value of the variant polypeptide is at least 1° C., 2° C., 3° C., 4° C., 5° C., 6° C., 7° C., 8° C., 9° C., 10° C., 11° C., 12° C., 13° C., 14° C., 15° C., 16° C., 17° C., 18° C., 19° C., or 20° C. greater than the Tₘ value of a parent polypeptide. In one embodiment, the variant polypeptide exhibits enhanced RNA guide binding specificity when the Tₘ value of the variant polypeptide is at least 8° C. greater than the Tₘ value of the parent polypeptide.

In some embodiments, the variant polypeptide comprises at least one alteration that enhances protein-RNA interactions, as compared to a parent polypeptide. In some embodiments, the variant polypeptide exhibits enhanced protein-RNA interactions, as compared to a parent polypeptide, at a temperature lower than about any one of 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 50° C., 51° C., 52° C., 53° C., 54° C., 55° C., 56° C., 57° C., 58° C., 59° C., 60° C. or 65° C. In some embodiments, the variant polypeptide exhibits enhanced protein-RNA interactions, as compared to a parent polypeptide, in a buffer having a pH in a range of about 7.3 to about 8.6 (e.g., about 7.5 to about 8.0, about 7.8 to 8.3, or about 8.0 to 8.6). In some embodiments, the variant polypeptide exhibits enhanced protein-RNA interactions, as compared to a parent polypeptide, when the Tₘ value of the variant polypeptide is at least 1° C., 2° C., 3° C., 4° C., 5° C., 6° C., 7° C., 8° C., 9° C., 10° C., 11° C., 12° C., 13° C., 14° C., 15° C., 16° C., 17° C., 18° C., 19° C., or 20° C. greater than the Tₘ value of a parent polypeptide. In one embodiment, the variant polypeptide exhibits enhanced protein-RNA interactions when the Tₘ value of the variant polypeptide is at least 8° C. greater than the Tₘ value of the parent polypeptide.

In some embodiments, the variant polypeptide comprises at least one alteration that enhances protein stability, as compared to a parent polypeptide. In some embodiments, the variant polypeptide exhibits enhanced protein stability, as compared to a parent polypeptide, at a temperature lower than about any one of 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 50° C., 51° C., 52° C., 53° C., 54° C., 55° C., 56° C., 57° C., 58° C., 59° C., 60° C. or 65° C. In some embodiments, the variant polypeptide exhibits enhanced protein stability, as compared to a parent polypeptide, in a buffer having a pH in a range of about 7.3 to about 8.6 (e.g., about 7.5 to about 8.0, about 7.8 to 8.3, or about 8.0 to 8.6). In some embodiments, the variant polypeptide exhibits enhanced protein stability, as compared to a parent polypeptide, when the Tₘ value of the variant polypeptide is at least 1° C., 2° C., 3° C., 4° C., 5° C., 6° C., 7° C., 8° C., 9° C., 10° C., 11° C., 12° C., 13° C., 14° C., 15° C., 16° C., 17° C., 18° C., 19° C., or 20° C. greater than the Tₘ value of a parent polypeptide. In one embodiment, the variant polypeptide exhibits enhanced protein stability when the Tₘ value of the variant polypeptide is at least 8° C. greater than the Tₘ value of the parent polypeptide.

In some embodiments, the variant polypeptide comprises at least one alteration that decreases dissociation from an RNA guide (e.g., binary complex dissociation), as compared to a parent polypeptide. In some embodiments, the variant polypeptide exhibits decreased dissociation from an RNA guide, as compared to a parent polypeptide, at a temperature lower than about any one of 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 50° C., 51° C., 52° C., 53º° C., 54° C., 55° C., 56° C., 57° C., 58° C., 59° C., 60° C. or 65° C. In some embodiments, the variant polypeptide exhibits decreased dissociation from an RNA guide, as compared to a parent polypeptide, in a buffer having a pH in a range of about 7.3 to about 8.6 (e.g., about 7.5 to about 8.0, about 7.8 to 8.3, or about 8.0 to 8.6). In some embodiments, the variant polypeptide exhibits decreased dissociation from an RNA guide, as compared to a parent polypeptide, when the Tₘ value of the variant polypeptide is at least 1° C., 2° C., 3° C., 4° C., 5° C., 6° C., 7° C., 8° C., 9° C., 10° C., 11° C., 12° C., 13° C., 14° C., 15° C., 16° C., 17° C., 18° C., 19° C., or 20° C. greater than the Tₘ value of a parent polypeptide. In one embodiment, the variant polypeptide exhibits decreased dissociation from an RNA guide when the Tₘ value of the variant polypeptide is at least 8° C. greater than the Tₘ value of the parent polypeptide. In some embodiments, the variant polypeptide exhibits decreased dissociation from an RNA guide, as compared to a parent polypeptide, over an incubation period of at least about any one of 10 mins, 15 mins, 20 mins, 25 mins, 30 mins, 35 mins, 40 mins, 45 mins, 50 mins, 55 mins, Ihr, 2 hr, 3 hr, 4 hr, or more hours. In some embodiments, a variant ribonucleoprotein (RNP) complex does not exchange the RNA guide with a different RNA.

In some embodiments, the variant polypeptide comprises at least one alteration that enhances ternary complex formation with an RNA guide and a target nucleic acid, as compared to a parent polypeptide. In some embodiments, the variant polypeptide exhibits enhanced ternary complex formation, as compared to a parent polypeptide, at a temperature lower than about any one of 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 50° C., 51° C., 52° C., 53° C., 54° C., 55° C., 56° C., 57° C., 58° C., 59° C., 60° C. or 65° C. In some embodiments, the variant polypeptide exhibits enhanced ternary complex formation, as compared to a parent polypeptide, in a buffer having a pH in a range of about 7.3 to about 8.6 (e.g., about 7.5 to about 8.0, about 7.8 to 8.3, or about 8.0 to 8.6). In some embodiments, the variant polypeptide exhibits enhanced ternary complex formation, as compared to a parent polypeptide, when the Tₘ value of the variant polypeptide is at least 1° C., 2° C., 3° C., 4° C., 5° C., 6° C., 7° C., 8° C., 9° C., 10° C., 11° C., 12° C., 13° C., 14° C., 15° C., 16° C., 17° C., 18° C., 19° C., or 20° C. greater than the Tₘ value of a parent polypeptide. In one embodiment, the variant polypeptide exhibits enhanced ternary complex formation when the Tₘ value of the variant polypeptide is at least 8° C. greater than the Tₘ value of the parent polypeptide.

In some embodiments, the variant polypeptide comprises at least one alteration such that a binary complex comprising the variant polypeptide (e.g., a variant binary complex) exhibits enhanced binding affinity to a target nucleic acid, as compared to a parent binary complex. In some embodiments, the variant binary complex exhibits enhanced binding affinity to a target nucleic acid, as compared to a parent binary complex, at a temperature lower than about any one of 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 50° C., 51° C., 52° C., 53° C., 54° C., 55° C., 56° C., 57° C., 58° C., 59° C., 60° C. or 65° C. In some embodiments, the variant binary complex exhibits enhanced binding affinity to a target nucleic acid, as compared to a parent binary complex, in a buffer having a pH in a range of about 7.3 to about 8.6 (e.g., about 7.5 to about 8.0, about 7.8 to 8.3, or about 8.0 to 8.6). In some embodiments, the variant binary complex exhibits enhanced binding affinity to a target nucleic acid, as compared to a parent binary complex, when the Tₘ value of the variant binary complex is at least 1° C., 2° C., 3° C., 4° C., 5° C., 6° C., 7° C., 8° C., 9° C., 10° C., 11° C., 12° C., 13° C., 14° C., 15° C., 16° C., 17° C., 18° C., 19° C., or 20° C. greater than the Tₘ value of a parent binary complex. In one embodiment, the variant binary complex exhibits enhanced binding affinity to a target nucleic acid when the Tₘ value of the variant binary complex is at least 8° C. greater than the Tₘ value of the parent binary complex.

In some embodiments, the variant polypeptide comprises at least one alteration such that a binary complex comprising the variant polypeptide (e.g., a variant binary complex) exhibits enhanced on-target binding activity, as compared to a parent binary complex. In some embodiments, the variant binary complex exhibits enhanced on-target binding activity, as compared to a parent binary complex, at a temperature lower than about any one of 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 50° C., 51° C., 52° C., 53° C., 54° C., 55° C., 56° C., 57° C., 58° C., 59° C., 60° C. or 65° C. In some embodiments, the variant binary complex exhibits enhanced on-target binding activity, as compared to a parent binary complex, in a buffer having a pH in a range of about 7.3 to about 8.6 (e.g., about 7.5 to about 8.0, about 7.8 to 8.3, or about 8.0 to 8.6). In some embodiments, the variant binary complex exhibits enhanced on-target binding activity, as compared to a parent binary complex, when the Tₘ value of the variant binary complex is at least 1° C., 2° C., 3º° C., 4° C., 5° C., 6° C., 7º° C., 8° C., 9° C., 10° C., 11° C., 12° C., 13° C., 14° C., 15° C., 16° C., 17° C., 18° C., 19° C., or 20° C. greater than the Tₘ value of a parent binary complex. In one embodiment, the variant binary complex exhibits enhanced on-target binding activity when the Tₘ value of the variant binary complex is at least 8° C. greater than the Tₘ value of the parent binary complex.

In some embodiments, the variant polypeptide comprises at least one alteration such that a binary complex comprising the variant polypeptide (e.g., a variant binary complex) exhibits enhanced on-target binding specificity, as compared to a parent binary complex. In some embodiments, the variant binary complex exhibits enhanced on-target binding specificity, as compared to a parent binary complex, at a temperature lower than about any one of 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 50° C., 51° C., 52° C., 53° C., 54° C., 55° C., 56° C., 57° C., 58° C., 59° C., 60° C. or 65° C. In some embodiments, the variant binary complex exhibits enhanced on-target binding specificity, as compared to a parent binary complex, in a buffer having a pH in a range of about 7.3 to about 8.6 (e.g., about 7.5 to about 8.0, about 7.8 to 8.3, or about 8.0 to 8.6). In some embodiments, the variant binary complex exhibits enhanced on-target binding specificity, as compared to a parent binary complex, when the Tₘ value of the variant binary complex is at least 1° C., 2° C., 3° C., 4° C., 5° C., 6° C., 7° C., 8° C., 9° C., 10° C., 11° C., 12° C., 13° C., 14° C., 15° C., 16° C., 17° C., 18° C., 19° C., or 20° C. greater than the Tₘ value of a parent binary complex. In one embodiment, the variant binary complex exhibits enhanced on-target binding specificity when the Tₘ value of the variant binary complex is at least 8° C. greater than the Tₘ value of the parent binary complex.

In some embodiments, the variant polypeptide comprises at least one alteration such that a binary complex comprising the variant polypeptide (e.g., a variant binary complex) exhibits decreased off-target binding to a non-target nucleic acid, as compared to a parent binary complex. In some embodiments, the variant binary complex exhibits decreased off-target binding to a non-target nucleic acid, as compared to a parent binary complex, at a temperature lower than about any one of 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 50° C., 51° C., 52° C., 53° C., 54° C., 55° C., 56° C., 57° C., 58° C., 59° C., 60° C. or 65° C. In some embodiments, the variant binary complex exhibits decreased off-target binding to a non-target nucleic acid, as compared to a parent binary complex, in a buffer having a pH in a range of about 7.3 to about 8.6 (e.g., about 7.5 to about 8.0, about 7.8 to 8.3, or about 8.0 to 8.6). In some embodiments, the variant binary complex exhibits decreased off-target binding to a non-target nucleic acid, as compared to a parent binary complex, when the Tₘ value of the variant polypeptide is at least 1° C., 2° C., 3° C., 4° C., 5° C., 6° C., 7° C., 8° C., 9° C., 10° C., 11° C., 12° C., 13° C., 14° C., 15° C., 16° C., 17° C., 18° C., 19° C., or 20° C. greater than the Tₘ value of a parent polypeptide. In one embodiment, the variant binary complex exhibits decreased off-target binding to a non-target nucleic acid when the Tₘ value of the variant binary complex is at least 8° C. greater than the Tₘ value of the parent polypeptide.

In some embodiments, the variant polypeptide comprises at least one alteration such that a binary complex comprising the variant polypeptide (e.g., a variant binary complex) exhibits decreased dissociation from the target nucleic acid, as compared to a parent binary complex. In some embodiments, the variant binary complex exhibits decreased dissociation from the target nucleic acid, as compared to a parent binary complex, at a temperature lower than about any one of 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 50° C., 51° C., 52° C., 53° C., 54° C., 55° C., 56° C., 57° C., 58° C., 59° C., 60° C. or 65° C. In some embodiments, the variant binary complex exhibits decreased dissociation from the target nucleic acid, as compared to a parent binary complex, in a buffer having a pH in a range of about 7.3 to about 8.6 (e.g., about 7.5 to about 8.0, about 7.8 to 8.3, or about 8.0 to 8.6). In some embodiments, the variant binary complex exhibits decreased dissociation from the target nucleic acid, as compared to a parent binary complex, when the Tₘ value of the variant polypeptide is at least 1° C., 2° C., 3° C., 4° C., 5° C., 6° C., 7° C., 8° C., 9° C., 10° C., 11° C., 12° C., 13° C., 14° C., 15° C., 16° C., 17° C., 18° C., 19° C., or 20° C. greater than the Tₘ value of a parent polypeptide. In one embodiment, the variant binary complex exhibits decreased dissociation from the target nucleic acid when the Tₘ value of the variant binary complex is at least 8° C. greater than the Tₘ value of the parent polypeptide.

In some embodiments, the variant polypeptide comprises at least one alteration such that a ternary complex comprising the variant polypeptide (e.g., a variant ternary complex) exhibits enhanced stability, as compared to a parent ternary complex. In some embodiments, the variant ternary complex exhibits enhanced stability, as compared to a parent ternary complex, at a temperature lower than about any one of 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 50° C., 51° C., 52° C., 53° C., 54° C., 55° C., 56° C., 57° C., 58° C., 59° C., 60° C. or 65° C. In some embodiments, the variant ternary complex exhibits enhanced stability, as compared to a parent ternary complex, in a buffer having a pH in a range of about 7.3 to about 8.6 (e.g., about 7.5 to about 8.0, about 7.8 to 8.3, or about 8.0 to 8.6). In some embodiments, the variant ternary complex exhibits enhanced stability, as compared to a parent ternary complex, when the Tₘ value of the variant ternary complex is at least 1° C., 2° C., 3° C., 4° C., 5° C., 6° C., 7° C., 8° C., 9° C., 10° C., 11° C., 12° C., 13° C., 14° C., 15° C., 16° C., 17° C., 18° C., 19° C., or 20° C. greater than the Tₘ value of a parent ternary complex. In one embodiment, the variant ternary complex exhibits enhanced stability when the Tₘ value of the variant ternary complex is at least 8° C. greater than the Tₘ value of the parent ternary complex.

In some embodiments, at least one alteration is introduced into the parent polypeptide of SEQ ID NO: 3 to produce a variant polypeptide that exhibits (a) decreased enzymatic activity and (b) enhanced RNA affinity relative to the parent polypeptide of SEQ ID NO: 3. In some embodiments, at least one alteration is introduced into the parent polypeptide of SEQ ID NO: 3 to produce a variant polypeptide that exhibits (a) increased enzymatic activity and (b) enhanced RNA affinity, relative to the parent polypeptide of SEQ ID NO: 3. In some embodiments, at least one alteration is introduced into the parent polypeptide of SEQ ID NO: 3 to produce a variant polypeptide that exhibits (a) retained enzymatic activity and (b) enhanced RNA affinity, relative to the parent polypeptide of SEQ ID NO: 3. In some embodiments, the variant polypeptide having a feature as described herein comprises an amino acid sequence having at least about 60%, 65%, 70%, 75%, 80%, 85%, 90%, 91%, 92%, 93%, 94%, 95%, 96%, 97%, 98%, 99%, or 100% identity to a polypeptide comprising a substitution of Table 2 and/or substitutions of Table 3.

In some embodiments, at least one alteration is introduced into the parent polypeptide of SEQ ID NO: 3 to produce a variant polypeptide that exhibits (a) decreased enzymatic activity and (b) enhanced binary complex formation relative to the parent polypeptide of SEQ ID NO: 3. In some embodiments, at least one alteration is introduced into the parent polypeptide of SEQ ID NO: 3 to produce a variant polypeptide that exhibits (a) increased enzymatic activity and (b) enhanced binary complex formation, relative to the parent polypeptide of SEQ ID NO: 3. In some embodiments, at least one alteration is introduced into the parent polypeptide of SEQ ID NO: 3 to produce a variant polypeptide that exhibits (a) retained enzymatic activity and (b) enhanced binary complex formation, relative to the parent polypeptide of SEQ ID NO: 3. In some embodiments, the variant polypeptide having a feature as described herein comprises an amino acid sequence having at least about 60%, 65%, 70%, 75%, 80%, 85%, 90%, 91%, 92%. 93%, 94%, 95%, 96%, 97%, 98%, 99%, or 100% identity to a polypeptide comprising a substitution of Table 2 and/or substitutions of Table 3.

In some embodiments, at least one alteration is introduced into the parent polypeptide of SEQ ID NO: 3 to produce a variant polypeptide that exhibits (a) decreased enzymatic activity and (b) enhanced RNA guide binding activity relative to the parent polypeptide of SEQ ID NO: 3. In some embodiments, at least one alteration is introduced into the parent polypeptide of SEQ ID NO: 3 to produce a variant polypeptide that exhibits (a) increased enzymatic activity and (b) enhanced RNA guide binding activity, relative to the parent polypeptide of SEQ ID NO: 3. In some embodiments, at least one alteration is introduced into the parent polypeptide of SEQ ID NO: 3 to produce a variant polypeptide that exhibits (a) retained enzymatic activity and (b) enhanced RNA guide binding activity, relative to the parent polypeptide of SEQ ID NO: 3. In some embodiments, the variant polypeptide having a feature as described herein comprises an amino acid sequence having at least about 60%, 65%, 70%, 75%, 80%, 85%, 90%, 91%, 92%. 93%, 94%, 95%, 96%, 97%, 98%, 99%, or 100% identity to a polypeptide comprising a substitution of Table 2 and/or substitutions of Table 3.

In some embodiments, at least one alteration is introduced into the parent polypeptide of SEQ ID NO: 3 to produce a variant polypeptide that exhibits (a) decreased enzymatic activity and (b) enhanced RNA guide binding specificity relative to the parent polypeptide of SEQ ID NO: 3. In some embodiments, at least one alteration is introduced into the parent polypeptide of SEQ ID NO: 3 to produce a variant polypeptide that exhibits (a) increased enzymatic activity and (b) enhanced RNA guide binding specificity, relative to the parent polypeptide of SEQ ID NO: 3. In some embodiments, at least one alteration is introduced into the parent polypeptide of SEQ ID NO: 3 to produce a variant polypeptide that exhibits (a) retained enzymatic activity and (b) enhanced RNA guide binding specificity, relative to the parent polypeptide of SEQ ID NO: 3. In some embodiments, the variant polypeptide having a feature as described herein comprises an amino acid sequence having at least about 60%, 65%, 70%, 75%, 80%, 85%, 90%, 91%, 92%, 93%, 94%, 95%, 96%, 97%, 98%, 99%, or 100% identity to a polypeptide comprising a substitution of Table 2 and/or substitutions of Table 3.

In some embodiments, at least one alteration is introduced into the parent polypeptide of SEQ ID NO: 3 to produce a variant polypeptide that exhibits (a) decreased enzymatic activity and (b) enhanced protein-RNA interactions relative to the parent polypeptide of SEQ ID NO: 3. In some embodiments, at least one alteration is introduced into the parent polypeptide of SEQ ID NO: 3 to produce a variant polypeptide that exhibits (a) increased enzymatic activity and (b) enhanced protein-RNA interactions, relative to the parent polypeptide of SEQ ID NO: 3. In some embodiments, at least one alteration is introduced into the parent polypeptide of SEQ ID NO: 3 to produce a variant polypeptide that exhibits (a) retained enzymatic activity and (b) enhanced protein-RNA interactions, relative to the parent polypeptide of SEQ ID NO: 3. In some embodiments, the variant polypeptide having a feature as described herein comprises an amino acid sequence having at least about 60%, 65%, 70%, 75%, 80%, 85%, 90%, 91%, 92%, 93%, 94%, 95%, 96%, 97%, 98%, 99%, or 100% identity to a polypeptide comprising a substitution of Table 2 and/or substitutions of Table 3.

In some embodiments, at least one alteration is introduced into the parent polypeptide of SEQ ID NO: 3 to produce a variant polypeptide that exhibits (a) decreased enzymatic activity and (b) enhanced protein stability relative to the parent polypeptide of SEQ ID NO: 3. In some embodiments, at least one alteration is introduced into the parent polypeptide of SEQ ID NO: 3 to produce a variant polypeptide that exhibits (a) increased enzymatic activity and (b) enhanced protein stability, relative to the parent polypeptide of SEQ ID NO: 3. In some embodiments, at least one alteration is introduced into the parent polypeptide of SEQ ID NO: 3 to produce a variant polypeptide that exhibits (a) retained enzymatic activity and (b) enhanced protein stability, relative to the parent polypeptide of SEQ ID NO: 3. In some embodiments, the variant polypeptide having a feature as described herein comprises an amino acid sequence having at least about 60%, 65%, 70%, 75%, 80%, 85%, 90%, 91%, 92%, 93%, 94%, 95%, 96%, 97%, 98%, 99%, or 100% identity to a polypeptide comprising a substitution of Table 2 and/or substitutions of Table 3.

In some embodiments, at least one alteration is introduced into the parent polypeptide of SEQ ID NO: 3 to produce a variant polypeptide that exhibits (a) decreased enzymatic activity and (b) decreased dissociation from an RNA guide relative to the parent polypeptide of SEQ ID NO: 3. In some embodiments, at least one alteration is introduced into the parent polypeptide of SEQ ID NO: 3 to produce a variant polypeptide that exhibits (a) increased enzymatic activity and (b) decreased dissociation from an RNA guide, relative to the parent polypeptide of SEQ ID NO: 3. In some embodiments, at least one alteration is introduced into the parent polypeptide of SEQ ID NO: 3 to produce a variant polypeptide that exhibits (a) retained enzymatic activity and (b) decreased dissociation from an RNA guide, relative to the parent polypeptide of SEQ ID NO: 3. In some embodiments, the variant polypeptide having a feature as described herein comprises an amino acid sequence having at least about 60%, 65%, 70%, 75%, 80%, 85%, 90%. 91%, 92%, 93%, 94%, 95%, 96%, 97%, 98%, 99%, or 100% identity to a polypeptide comprising a substitution of Table 2 and/or substitutions of Table 3.

In some embodiments, at least one alteration is introduced into the parent polypeptide of SEQ ID NO: 3 to produce a variant polypeptide that exhibits (a) decreased enzymatic activity and (b) enhanced ternary complex formation relative to the parent polypeptide of SEQ ID NO: 3. In some embodiments, at least one alteration is introduced into the parent polypeptide of SEQ ID NO: 3 to produce a variant polypeptide that exhibits (a) increased enzymatic activity and (b) enhanced ternary complex formation, relative to the parent polypeptide of SEQ ID NO: 3. In some embodiments, at least one alteration is introduced into the parent polypeptide of SEQ ID NO: 3 to produce a variant polypeptide that exhibits (a) retained enzymatic activity and (b) enhanced ternary complex formation, relative to the parent polypeptide of SEQ ID NO: 3. In some embodiments, the variant polypeptide having a feature as described herein comprises an amino acid sequence having at least about 60%, 65%, 70%, 75%, 80%, 85%, 90%, 91%, 92%. 93%, 94%, 95%, 96%, 97%, 98%, 99%, or 100% identity to a polypeptide comprising a substitution of Table 2 and/or substitutions of Table 3.

In some embodiments, at least one alteration is introduced into the parent polypeptide of SEQ ID NO: 3 to produce a variant polypeptide that forms a variant binary complex exhibiting (a) decreased enzymatic activity and (b) enhanced binding affinity to a target nucleic acid, relative to a parent binary complex comprising the polypeptide of SEQ ID NO: 3. In some embodiments, at least one alteration is introduced into the parent polypeptide of SEQ ID NO: 3 to produce a variant polypeptide that forms a variant binary complex exhibiting (a) increased enzymatic activity and (b) enhanced binding affinity to a target nucleic acid, relative to a parent binary complex comprising the polypeptide of SEQ ID NO: 3. In some embodiments, at least one alteration is introduced into the parent polypeptide of SEQ ID NO: 3 to produce a variant polypeptide that forms a variant binary complex that exhibits (a) retained enzymatic activity and (b) enhanced binding affinity to a target nucleic acid, relative to a parent binary complex comprising the polypeptide of SEQ ID NO: 3. In some embodiments, the variant polypeptide having a feature as described herein comprises an amino acid sequence having at least about 60%, 65%, 70%, 75%. 80%, 85%, 90%, 91%, 92%, 93%, 94%, 95%, 96%, 97%, 98%, 99%, or 100% identity to a polypeptide comprising a substitution of Table 2 and/or substitutions of Table 3.

In some embodiments, at least one alteration is introduced into the parent polypeptide of SEQ ID NO: 3 to produce a variant polypeptide that forms a variant binary complex exhibiting (a) decreased enzymatic activity and (b) enhanced on-target binding activity, relative to a parent binary complex comprising the polypeptide of SEQ ID NO: 3. In some embodiments, at least one alteration is introduced into the parent polypeptide of SEQ ID NO: 3 to produce a variant polypeptide that forms a variant binary complex exhibiting (a) increased enzymatic activity and (b) enhanced on-target binding activity, relative to a parent binary complex comprising the polypeptide of SEQ ID NO: 3. In some embodiments, at least one alteration is introduced into the parent polypeptide of SEQ ID NO: 3 to produce a variant polypeptide that forms a variant binary complex that exhibits (a) retained enzymatic activity and (b) enhanced on-target binding activity, relative to a parent binary complex comprising the polypeptide of SEQ ID NO: 3. In some embodiments, the variant polypeptide having a feature as described herein comprises an amino acid sequence having at least about 60%, 65%, 70%, 75%, 80%, 85%, 90%, 91%, 92%, 93%, 94%, 95%, 96%. 97%, 98%, 99%, or 100% identity to a polypeptide comprising a substitution of Table 2 and/or substitutions of Table 3.

In some embodiments, at least one alteration is introduced into the parent polypeptide of SEQ ID NO: 3 to produce a variant polypeptide that forms a variant binary complex exhibiting (a) decreased enzymatic activity and (b) enhanced on-target binding specificity, relative to a parent binary complex comprising the polypeptide of SEQ ID NO: 3. In some embodiments, at least one alteration is introduced into the parent polypeptide of SEQ ID NO: 3 to produce a variant polypeptide that forms a variant binary complex exhibiting (a) increased enzymatic activity and (b) enhanced on-target binding specificity, relative to a parent binary complex comprising the polypeptide of SEQ ID NO: 3. In some embodiments, at least 20) one alteration is introduced into the parent polypeptide of SEQ ID NO: 3 to produce a variant polypeptide that forms a variant binary complex that exhibits (a) retained enzymatic activity and (b) enhanced on-target binding specificity, relative to a parent binary complex comprising the polypeptide of SEQ ID NO: 3. In some embodiments, the variant polypeptide having a feature as described herein comprises an amino acid sequence having at least about 60%, 65%, 70%, 75%, 80%, 85%, 90%, 91%, 92%, 93%, 94%, 95%, 96%. 97%, 98%, 99%, or 100% identity to a polypeptide comprising a substitution of Table 2 and/or substitutions of Table 3.

In some embodiments, at least one alteration is introduced into the parent polypeptide of SEQ ID NO: 3 to produce a variant polypeptide that forms a variant binary complex exhibiting (a) decreased enzymatic activity and (b) decreased off-target binding to a non-target nucleic acid, relative to a parent binary complex comprising the polypeptide of SEQ ID NO: 3. In some embodiments, at least one alteration is introduced into the parent polypeptide of SEQ ID NO: 3 to produce a variant polypeptide that forms a variant binary complex exhibiting (a) increased enzymatic activity and (b) decreased off-target binding to a non-target nucleic acid, relative to a parent binary complex comprising the polypeptide of SEQ ID NO: 3. In some embodiments, at least one alteration is introduced into the parent polypeptide of SEQ ID NO: 3 to produce a variant polypeptide that forms a variant binary complex exhibiting (a) retained enzymatic activity and (b) decreased off-target binding to a non-target nucleic acid, relative to a parent binary complex comprising the polypeptide of SEQ ID NO: 3. In some embodiments, the variant polypeptide having a feature as described herein comprises an amino acid sequence having at least about 60%, 65%, 70%, 75%, 80%, 85%, 90%, 91%, 92%, 93%, 94%, 95%, 96%, 97%, 98%, 99%, or 100% identity to a polypeptide comprising a substitution of Table 2 and/or substitutions of Table 3.

In some embodiments, at least one alteration is introduced into the parent polypeptide of SEQ ID NO: 3 to produce a variant polypeptide that forms a variant binary complex exhibiting (a) decreased enzymatic activity and (b) decreased dissociation from the target nucleic acid, relative to a parent binary complex comprising the polypeptide of SEQ ID NO: 3. In some embodiments, at least one alteration is introduced into the parent polypeptide of SEQ ID NO: 3 to produce a variant polypeptide that forms a variant binary complex exhibiting (a) increased enzymatic activity and (b) decreased dissociation from the target nucleic acid, relative to a parent binary complex comprising the polypeptide of SEQ ID NO: 3. In some embodiments, at least one alteration is introduced into the parent polypeptide of SEQ ID NO: 3 to produce a variant polypeptide that forms a variant binary complex exhibiting (a) retained enzymatic activity and (b) decreased dissociation from the target nucleic acid, relative to a parent binary complex comprising the polypeptide of SEQ ID NO: 3. In some embodiments, the variant polypeptide having a feature as described herein comprises an amino acid sequence having at least about 60%, 65%, 70%, 75%, 80%, 85%, 90%, 91%, 92%, 93%, 94%, 95%, 96%, 97%, 98%, 99%, or 100% identity to a polypeptide comprising a substitution of Table 2 and/or substitutions of Table 3.

### RNA Guide

In some embodiments, a composition or complex as described herein comprises a targeting moiety (e.g., an RNA guide, antisense, oligonucleotides, peptide oligonucleotide conjugates) that binds the target nucleic acid and interacts with the variant polypeptide. The targeting moiety may bind a target nucleic acid (e.g., with specific binding affinity to the target nucleic acid).

In some embodiments, the targeting moiety comprises, or is, an RNA guide. In some embodiments, the RNA guide directs the variant polypeptide described herein to a particular nucleic acid sequence. Those skilled in the art reading the below examples of particular kinds of RNA guides will understand that, in some embodiments, an RNA guide is site-specific. That is, in some embodiments, an RNA guide associates specifically with one or more target nucleic acid sequences (e.g., specific DNA or genomic DNA sequences) and not to non-targeted nucleic acid sequences (e.g., non-specific DNA or random sequences).

In some embodiments, the composition as described herein comprises an RNA guide that associates with the variant polypeptide described herein and directs the variant polypeptide to a target nucleic acid sequence (e.g., DNA).

The RNA guide may target (e.g., associate with, be directed to, contact, or bind) one or more nucleotides of a target sequence, e.g., a site-specific sequence or a site-specific target. In some embodiments, the variant ribonucleoprotein (e.g., variant CRISPR nuclease polypeptide plus an RNA guide) is activated upon binding to a target nucleic acid that is complementary to a DNA-targeting sequence in the RNA guide (e.g., a sequence-specific substrate or target nucleic acid).

In some embodiments, an RNA guide comprises a spacer having a length of from about 11 nucleotides to about 100 nucleotides. For example, the DNA-targeting segment can have a length of from about 11 nucleotides to about 80 nucleotides, from about 11 nucleotides to about 50 nucleotides, from about 11 nucleotides to about 40 nucleotides, from about 11 nucleotides to about 30 nucleotides, from about 11 nucleotides to about 25 nucleotides, from about 11 nucleotides to about 20 nucleotides, or from about 11 nucleotides to about 19 nucleotides. For example, the spacer can have a length of from about 19 nucleotides to about 20 nucleotides, from about 19 nucleotides to about 25 nucleotides, from about 19 nucleotides to about 30 nucleotides, from about 19 nucleotides to about 35 nucleotides, from about 19 nucleotides to about 40 nucleotides, from about 19 nucleotides to about 45 nucleotides, from about 19 nucleotides to about 50 nucleotides, from about 19 nucleotides to about 60 nucleotides, from about 19 nucleotides to about 70 nucleotides, from about 19 nucleotides to about 80 nucleotides, from about 19 nucleotides to about 90 nucleotides, from about 19 nucleotides to about 100 nucleotides, from about 20 nucleotides to about 25 nucleotides, from about 20 nucleotides to about 30 nucleotides, from about 20 nucleotides to about 35 nucleotides, from about 20 nucleotides to about 40 nucleotides, from about 20 nucleotides to about 45 nucleotides, from about 20 nucleotides to about 50 nucleotides, from about 20 nucleotides to about 60 nucleotides, from about 20 nucleotides to about 70 nucleotides, from about 20 nucleotides to about 80 nucleotides, from about 20 nucleotides to about 90 nucleotides, or from about 20 nucleotides to about 100 nucleotides.

In some embodiments, the spacer of the RNA guide may be generally designed to have a length of between 11 and 50 nucleotides (e.g., 11, 12, 13, 14, 15, 16, 17, 18, 19, 20, 21, 22, 23, 24, 25, 26, 27, 28, 29, 30, 31, 32, 33, 34, or 35 nucleotides) and be complementary to a specific target nucleic acid sequence. In some particular embodiments, the RNA guide may be designed to be complementary to a specific DNA strand, e.g., of a genomic locus. In some embodiments, the DNA targeting sequence is designed to be complementary to a specific DNA strand, e.g., of a genomic locus.

The RNA guide may be substantially identical to a complementary strand of a reference nucleic acid sequence. In some embodiments, the RNA guide comprises a sequence having least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 99.5% sequence identity to a complementary strand of a reference nucleic acid sequence, e.g., target nucleic acid. The percent identity between two such nucleic acids can be determined manually by inspection of the two optimally aligned nucleic acid sequences or by using software programs or algorithms (e.g., BLAST, ALIGN, CLUSTAL) using standard parameters.

In some embodiments, the RNA guide has at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 99.5% sequence identity to a complementary strand of a target nucleic acid.

In some embodiments, the RNA guide comprises a spacer that is a length of between 11 and 50 nucleotides (e.g., 11, 12, 13, 14, 15, 16, 17, 18, 19, 20, 21, 22, 23, 24, 25, 26, 27, 28, 29, 30, 31, 32, 33, 34, or 35 nucleotides) and at least 80%, at least 90%, at least 95%, at least 96%, at least 97%, at least 98%, at least 99% complementary to a target nucleic acid. In some embodiments, the RNA guide comprises a sequence at least 80%, at least 90%, at least 95%, at least 96%, at least 97%, at least 98%, at least 99% complementary to a target DNA sequence. In some embodiments, the RNA guide comprises a sequence at least 80%, at least 90%, at least 95%, at least 96%, at least 97%, at least 98%, at least 99% complementary to a target genomic sequence. In some embodiments, the RNA guide comprises a sequence, e.g., RNA sequence, that is a length of up to 50 and at least 80%, at least 90%, at least 95%, at least 96%, at least 97%, at least 98%, at least 99% complementary to a target nucleic acid. In some embodiments, the RNA guide comprises a sequence at least 80%, at least 90%, at least 95%, at least 96%, at least 97%, at least 98%, at least 99% complementary to a target DNA sequence. In some embodiments, the RNA guide comprises a sequence at least 80%, at least 90%, at least 95%, at least 96%, at least 97%, at least 98%, at least 99% complementary to a target genomic sequence.

In certain embodiments, the RNA guide includes, consists essentially of, or comprises a direct repeat sequence linked to a DNA targeting sequence. In some embodiments, the RNA guide includes a direct repeat sequence and a DNA targeting sequence or a direct repeat-DNA targeting sequence-direct repeat sequence. In some embodiments, the RNA guide includes a truncated direct repeat sequence and a DNA targeting sequence, which is typical of processed or mature crRNA. In some embodiments, the variant polypeptide described herein forms a complex with the RNA guide, and the RNA guide directs the complex to associate with site-specific target nucleic acid that is complementary to at least a portion of the RNA guide.

In some embodiments, the direct repeat sequence is at least 90% identical to a sequence set forth in Table 4 or a portion of a sequence set forth in Table 4. In some embodiments, the direct repeat sequence is at least 95% identical to a sequence set forth in Table 4 or a portion of a sequence set forth in Table 4. In some embodiments, the direct repeat sequence is identical to a sequence set forth in Table 4 or a portion of a sequence set forth in Table 4.

TABLE 4
Direct repeat sequences.

| Sequence | |
|---|---|
| identifier | Direct Repeat Sequence |
| SEQ ID NO: 4 | CUUGUUGUAUAUGUCCUUUUAUAGGUAUUAAACAAC |
| SEQ ID NO: 5 | CUUGUUGUAUAUACGCUUUUAUAGGUAUUAAACAAC |
| SEQ ID NO: 6 | CUUGUUGUAUAUACGCUUUUAUAGGUAUUGAACAAC |
| SEQ ID NO: 7 | CUUGUUGUAUAUAUCCUUUUAUAGAUAUUAAACAGC |
| SEQ ID NO: 8 | CUUGUUGUAUAUAUCCUUUUAUAGAUGUUGAACAAC |
| SEQ ID NO: 9 | CUUGUUGUAUAUAUCCUUUUAUAGGUGUUGAACAAC |
| SEQ ID NO: 10 | CUUGUUGUAUAUAUCCUUUUAUGGGUGUAUAACAAC |
| SEQ ID NO: 11 | CUUGUUGUAUAUGUCCUUUUAUAGGUAUUUGAACAA |
| | C |
| SEQ ID NO: 12 | CUUGUUGUAUAUGUCUUUUUAUAGGUAUUGAACAAC |
| SEQ ID NO: 13 | CUUGUUGUGUAUAUCCUUUUAUAGGUAUUGAACAAC |

In some embodiments, the direct repeat comprises a sequence set forth as CUUGUUGUNIUAU

(SEQ ID NO: 14), wherein N₁ is A or G. In some embodiments, the direct repeat comprises a sequence set forth as UUUUAUNIGN2UN3U (SEQ ID NO: 15), wherein N₁ is A or G, N₂ is A or G, and N₃ is A or G.

In some embodiments, the composition or complex described herein includes one or more (e.g., two, three, four, five, six, seven, eight, or more) RNA guides, e.g., a plurality of RNA guides.

In some embodiments, the RNA guide has an architecture similar to, for example International Publication Nos. WO 2014/093622 and WO 2015/070083, the entire contents of each of which are incorporated herein by reference.

Unless otherwise noted, all compositions and complexes and polypeptides provided herein are made in reference to the active level of that composition or complex or polypeptide, and are exclusive of impurities, for example, residual solvents or by-products, which may be present in commercially available sources. Enzymatic component weights are based on total active protein. All percentages and ratios are calculated by weight unless otherwise indicated. All percentages and ratios are calculated based on the total composition unless otherwise indicated. In the exemplified composition, the enzymatic levels are expressed by pure enzyme by weight of the total composition and unless otherwise specified, the ingredients are expressed by weight of the total compositions.

### Modifications

The RNA guide or any of the nucleic acid sequences encoding the variant polypeptides may include one or more covalent modifications with respect to a reference sequence, in particular the parent polyribonucleotide, which are included within the scope of this invention.

Exemplary modifications can include any modification to the sugar, the nucleobase, the internucleoside linkage (e.g., to a linking phosphate/to a phosphodiester linkage/to the phosphodiester backbone), and any combination thereof. Some of the exemplary modifications provided herein are described in detail below.

The RNA guide or any of the nucleic acid sequences encoding components of the variant polypeptides may include any useful modification, such as to the sugar, the nucleobase, or the internucleoside linkage (e.g., to a linking phosphate/to a phosphodiester linkage/to the phosphodiester backbone). One or more atoms of a pyrimidine nucleobase may be replaced or substituted with optionally substituted amino, optionally substituted thiol, optionally substituted alkyl (e.g., methyl or ethyl), or halo (e.g., chloro or fluoro). In certain embodiments, modifications (e.g., one or more modifications) are present in each of the sugar and the internucleoside linkage. Modifications may be modifications of ribonucleic acids (RNAs) to deoxyribonucleic acids (DNAs), threose nucleic acids (TNAs), glycol nucleic acids (GNAs), peptide nucleic acids (PNAs), locked nucleic acids (LNAs) or hybrids thereof). Additional modifications are described herein.

In some embodiments, the modification may include a chemical or cellular induced modification. For example, some nonlimiting examples of intracellular RNA modifications are described by Lewis and Pan in “RNA modifications and structures cooperate to guide RNA-protein interactions” from Nat Reviews Mol Cell Biol, 2017, 18:202-210.

Different sugar modifications, nucleotide modifications, and/or internucleoside linkages (e.g., backbone structures) may exist at various positions in the sequence. One of ordinary skill in the art will appreciate that the nucleotide analogs or other modification(s) may be located at any position(s) of the sequence, such that the function of the sequence is not substantially decreased. The sequence may include from about 1% to about 100% modified nucleotides (either in relation to overall nucleotide content, or in relation to one or more types of nucleotide, i.e, any one or more of A, G, U or C) or any intervening percentage (e.g., from 1% to 20%>, from 1% to 25%, from 1% to 50%, from 1% to 60%, from 1% to 70%, from 1% to 80%, from 1% to 90%, from 1% to 95%, from 10% to 20%, from 10% to 25%, from 10% to 50%, from 10% to 60%, from 10% to 70%, from 10% to 80%, from 10% to 90%, from 10% to 95%, from 10% to 100%, from 20% to 25%, from 20% to 50%, from 20% to 60%, from 20% to 70%, from 20% to 80%, from 20% to 90%, from 20% to 95%, from 20% to 100%, from 50% to 60%, from 50% to 70%, from 50% to 80%, from 50% to 90%, from 50% to 95%, from 50% to 100%, from 70% to 80%, from 70% to 90%, from 70% to 95%, from 70% to 100%, from 80% to 90%, from 80% to 95%, from 80% to 100%, from 90% to 95%, from 90% to 100%, and from 95% to 100%).

In some embodiments, sugar modifications (e.g., at the 2′ position or 4′ position) or replacement of the sugar at one or more ribonucleotides of the sequence may, as well as backbone modifications, include modification or replacement of the phosphodiester linkages. Specific examples of a sequence include, but are not limited to, sequences including modified backbones or no natural internucleoside linkages such as internucleoside modifications, including modification or replacement of the phosphodiester linkages. Sequences having modified backbones include, among others, those that do not have a phosphorus atom in the backbone. For the purposes of this application, and as sometimes referenced in the art, modified RNAs that do not have a phosphorus atom in their internucleoside backbone can also be considered to be oligonucleosides. In particular embodiments, a sequence will include ribonucleotides with a phosphorus atom in its internucleoside backbone.

Modified sequence backbones may include, for example, phosphorothioates, chiral phosphorothioates, phosphorodithioates, phosphotriesters, aminoalkylphosphotriesters, methyl and other 3′-alkylene phosphonates and chiral phosphonates, phosphinates, alkyl phosphonates such as phosphoramidates such as 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms are also included. In some embodiments, the sequence may be negatively or positively charged.

The modified nucleotides, which may be incorporated into the sequence, can be modified on the internucleoside linkage (e.g., phosphate backbone). Herein, in the context of the polynucleotide backbone, the phrases “phosphate” and “phosphodiester” are used interchangeably. Backbone phosphate groups can be modified by replacing one or more of the oxygen atoms with a different substituent. Further, the modified nucleosides and nucleotides can include the wholesale replacement of an unmodified phosphate moiety with another internucleoside linkage as described herein. Examples of modified phosphate groups include, but are not limited to, phosphorothioate, phosphoroselenates, boranophosphates, boranophosphate esters, hydrogen phosphonates, phosphoramidates, phosphorodiamidates, alkyl or aryl phosphonates, and phosphotriesters. Phosphorodithioates have both non-linking oxygens replaced by sulfur. The phosphate linker can also be modified by the replacement of a linking oxygen with nitrogen (bridged phosphoramidates), sulfur (bridged phosphorothioates), and carbon (bridged methylene-phosphonates).

The α-thio substituted phosphate moiety is provided to confer stability to RNA and DNA polymers through the unnatural phosphorothioate backbone linkages. Phosphorothioate DNA and RNA have increased nuclease resistance and subsequently a longer half-life in a cellular environment.

In specific embodiments, a modified nucleoside includes an alpha-thio-nucleoside (e.g., 5′-O-(1-thiophosphate)-adenosine, 5′-O-(1-thiophosphate)-cytidine (α-thio-cytidine), 5′-O-(1-thiophosphate)-guanosine. 5′-O-(1-thiophosphate)-uridine, or 5′-O-(1-thiophosphate)-pseudouridine).

Other internucleoside linkages that may be employed according to the present invention, including internucleoside linkages which do not contain a phosphorous atom, are described herein.

In some embodiments, the sequence may include one or more cytotoxic nucleosides. For example, cytotoxic nucleosides may be incorporated into sequence, such as bifunctional modification. Cytotoxic nucleoside may include, but are not limited to, adenosine arabinoside, 5-azacytidine, 4-thio-aracytidinc, cyclopentenylcytosine, cladribine, clofarabine, cytarabine, cytosine arabinoside, 1-(2-C-cyano-2-deoxy-beta-D-arabino-pentofuranosyl)-cytosine, decitabine, 5-fluorouracil, fludarabine, floxuridine, gemcitabine, a combination of tegafur and uracil, tegafur ((RS)-5-fluoro-1-(tetrahydrofuran-2-yl)pyrimidine-10) 2,4(1H.3H)-dione), troxacitabine, tezacitabine, 2-deoxy-2-methylidenecytidine (DMDC), and 6-mercaptopurine. Additional examples include fludarabine phosphate. N4-behenoyl-1-beta-D-arabinofuranosylcytosine. N4-octadecyl-1-beta-D-arabinofuranosylcytosine. N4-palmitoyl-1-(2-C-cyano-2-deoxy-beta-D-arabino-pentofuranosyl) cytosine, and P-4055 (cytarabine 5′-elaidic acid ester).

In some embodiments, the sequence includes one or more post-transcriptional modifications (e.g., capping, cleavage, polyadenylation, splicing, poly-A sequence, methylation, acylation, phosphorylation, methylation of lysine and arginine residues, acetylation, and nitrosylation of thiol groups and tyrosine residues, etc.). The one or more post-transcriptional modifications can be any post-transcriptional modification, such as any of the more than one hundred different nucleoside modifications that have been identified in RNA (Rozenski. J. Crain. P, and McCloskey. J. (1999). The RNA Modification Database: 1999 update. Nucl Acids Res 27: 196-197) In some embodiments, the first isolated nucleic acid comprises messenger RNA (mRNA). In some embodiments, the mRNA comprises at least one nucleoside selected from the group consisting of pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine. 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridinc, 4-thio-1-methyl-pseudouridinc, 2-thio-1-methyl-pseudouridinc, 1-methyl-1-deaza-pseudouridinc, 2-thio-1-methyl-1-deaza-pseudouridine, dihydrouridinc, dihydropseudouridine, 2-thio-dihydrouridine, 2-thio-dihydropseudouridine, 2-methoxyuridine, 2-methoxy-4-thio-uridine, 4-methoxy-pseudouridine, and 4-methoxy-2-thio-pseudouridine. In some embodiments, the mRNA comprises at least one nucleoside selected from the group consisting of 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, and 4-methoxy-1-methyl-pseudoisocytidine. In some embodiments, the mRNA comprises at least one nucleoside selected from the group consisting of 2-aminopurine, 2, 6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-methyladenosine, N6-isopenteny ladenosine, N6-(cis-2-methylthio-N6-(cis-hydroxyisopentenyl) adenosine, N6-hydroxyisopentenyl)adenosine, glycinylcarbamoyladenosine, N6-threonylcarbamoyladenosine, 2-methylthio-N6-threonyl carbamoy ladenosine, N6, N6-dimethyladenosine, 7-methyladenine, 2-methylthio-adenine, and 2-methoxy-adenine. In some embodiments, mRNA comprises at least one nucleoside selected from the group consisting of inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N₂-methylguanosine, N₂,N₂-dimethylguanosine, 8-oxo-guanosine, 7-methyl-8-oxo-guanosine, 1-methyl-6-thio-guanosine, N₂-methyl-6-thio-guanosine, and N₂,N₂-dimethyl-6-thio-guanosine.

The sequence may or may not be uniformly modified along the entire length of the molecule. For example, one or more or all types of nucleotide (e.g., naturally-occurring nucleotides, purine or pyrimidine, or any one or more or all of A. G. U. C. I, pU) may or may not be uniformly modified in the sequence, or in a given predetermined sequence region thereof. In some embodiments, the sequence includes a pseudouridine. In some embodiments, the sequence includes an inosine, which may aid in the immune system characterizing the sequence as endogenous versus viral RNAs. The incorporation of inosine may also mediate improved RNA stability/reduced degradation. See for example, Yu, Z. et al. (2015) RNA editing by ADARI marks dsRNA as “self”. Cell Res, 25, 1283-1284, which is incorporated by reference in its entirety.

### Target Nucleic Acid

The methods disclosed herein are applicable for a variety of target nucleic acids. In some embodiments, the target nucleic acid is a DNA, such as a DNA locus. In some embodiments, the target nucleic acid is an RNA, such as an RNA locus or mRNA. In some embodiments, the target nucleic acid is single-stranded (e.g., single-stranded DNA). In some embodiments, the target nucleic acid is double-stranded (e.g., double-stranded DNA). In some embodiments, the target nucleic acid comprises both single-stranded and double-stranded regions. In some embodiments, the target nucleic acid is linear. In some embodiments, the target nucleic acid is circular. In some embodiments, the target nucleic acid comprises one or more modified nucleotides, such as methylated nucleotides, damaged nucleotides, or nucleotides analogs. In some embodiments, the target nucleic acid is not modified.

The target nucleic acid may be of any length, such as about at least any one of 100 bp, 200 bp, 500 bp, 1000 bp, 2000 bp, 5000 bp, 10 kb, 20 kb, 50 kb, 100 kb, 200 kb, 500 kb, 1 Mb, or longer. The target nucleic acid may also comprise any sequence. In some embodiments, the target nucleic acid is GC-rich, such as having at least about any one of 40%, 45%, 50%, 55%, 60%, 65%, or higher GC content. In some embodiments, the target nucleic acid has a GC content of at least about 70%, 80%, or more. In some embodiments, the target nucleic acid is a GC-rich fragment in a non-GC-rich target nucleic acid. In some embodiments, the target nucleic acid is not GC-rich. In some embodiments, the target nucleic acid has one or more secondary structures or higher-order structures. In some embodiments, the target nucleic acid is not in a condensed state, such as in a chromatin, to render the target nucleic acid inaccessible by the variant polypeptide/RNA guide complex.

In some embodiments, the target nucleic acid is present in a cell. In some embodiments, the target nucleic acid is present in the nucleus of the cell. In some embodiments, the target nucleic acid is endogenous to the cell. In some embodiments, the target nucleic acid is a genomic DNA. In some embodiments, the target nucleic acid is a chromosomal DNA. In one embodiment, the target nucleic acid is an extrachromosomal nucleic acid. In some embodiments, the target nucleic acid is a protein-coding gene or a functional region thereof, such as a coding region, or a regulatory element, such as a promoter, enhancer, a 5′ or 3′ untranslated region, etc. In some embodiments, the target nucleic acid is a non-coding gene, such as transposon, miRNA, tRNA, ribosomal RNA, ribozyme, or lincRNA. In some embodiments, the target nucleic acid is a plasmid.

In some embodiments, the target nucleic acid is exogenous to a cell. In some embodiments, the target nucleic acid is a viral nucleic acid, such as viral DNA or viral RNA. In some embodiments, the target nucleic acid is a horizontally transferred plasmid. In some embodiments, the target nucleic acid is integrated in the genome of the cell. In some embodiments, the target nucleic acid is not integrated in the genome of the cell. In some embodiments, the target nucleic acid is a plasmid in the cell. In some embodiments, the target nucleic acid is present in an extrachromosomal array.

In some embodiments, the target nucleic acid is an isolated nucleic acid, such as an isolated DNA or an isolated RNA. In some embodiments, the target nucleic acid is present in a cell-free environment. In some embodiments, the target nucleic acid is an isolated vector, such as a plasmid. In some embodiments, the target nucleic acid is an ultrapure plasmid.

The target nucleic acid is a segment of the target nucleic acid that hybridizes to the RNA guide. In some embodiments, the target nucleic acid has only one copy of the target nucleic acid. In some embodiments, the target nucleic acid has more than one copy, such as at least about any one of 2, 3, 4, 5, 10, 100, or more copies of the target nucleic acid. For example, a target nucleic acid comprising a repeated sequence in a genome of a viral nucleic acid or a bacterium may be targeted by the variant ribonucleoprotein.

The target sequence is adjacent to a protospacer adjacent motif or PAM of the disclosure as described herein. The PAM may be immediately adjacent to the target sequence or, for example, within a small number (e.g., 1, 2, 3, 4, or 5) of nucleotides of the target sequence. In the case of a double-stranded target, the targeting moiety (e.g., an RNA guide) binds to a first strand of the target and a PAM sequence as described herein is present in the second, complementary strand. In such a case, the PAM sequence is immediately adjacent to (or within a small number, e.g., 1, 2, 3, 4, or 5 nucleotides of) a sequence in the second strand that is complementary to the sequence in the first strand to which the binding moiety binds.

In some embodiments, the sequence-specificity requires a complete match of the spacer sequence in the RNA guide to the non-PAM strand of a target nucleic acid. In other embodiments, the sequence specificity requires a partial (contiguous or non-contiguous) match of the spacer sequence in the RNA guide to the non-PAM strand of a target nucleic acid.

In some embodiments, the RNA guide or a complex comprising the RNA guide and a variant polypeptide described herein binds to a target nucleic acid at a sequence defined by the region of complementarity between the RNA guide and the target nucleic acid. In some embodiments, the PAM sequence described herein is located directly upstream of the target sequence of the target nucleic acid (e.g., directly 5′ of the target sequence). In some embodiments, the PAM sequence described herein is located directly 5′ of the target sequence on the non-spacer-complementary strand (e.g., non-target strand) of the target nucleic acid.

In some embodiments, PAMs corresponding to a variant polypeptide of the present invention include 5′-NTTR-3′, 5′-NTTN-3′, 5′-RTTR-3′, 5′-ATTR-3′, or 5′-RTTG-3′. As used herein, N's can each be any nucleotide (e.g., A. G. T, or C) or a subset thereof (e.g., R (A or G), Y (C or T), K (G or T), B (G, T, or C), H (A, C, or T). In some embodiments, the PAM comprises 5′-GTTA-3′, 5′-TTTG-3′, 5′-CTTG-3′, 5′-GTTG-3′, 5′-TTTA-3′, 5′-CTTA-3′, 5′-ATTG-3′, 5′-ATTA-3′, 5′-ACTG-3′, 5′-CATA-3′, 5′-TTGA-3′, or 5′-TATA-3′. In some embodiments, a binary complex comprising a variant polypeptide of the present invention binds to a target nucleic acid adjacent to a 5″-NTTR-3′, 5′-NTTN-3′, 5′-RTTR-3″, 5′-ATTR-3′, or 5′-RTTG-3′ sequence. In some embodiments, a binary complex comprising a variant polypeptide of the present invention binds to a target nucleic acid adjacent to a 5′-GTTA-3′, 5′-TTTG-3′, 5′-CTTG-3′, 5′-GTTG-3′, 5′-TTTA-3′, 5′-CTTA-3′, 5′-ATTG-3′, 5′-ATTA-3′, 5′-ACTG-3′, 5′-CATA-3′, 5′-TTGA-3′, or 5′-TATA-3′ sequence.

In some embodiments, the target nucleic acid is present in a readily accessible region of the target nucleic acid. In some embodiments, the target nucleic acid is in an exon of a target gene. In some embodiments, the target nucleic acid is across an exon-intron junction of a target gene. In some embodiments, the target nucleic acid is present in a non-coding region, such as a regulatory region of a gene. In some embodiments, wherein the target nucleic acid is exogenous to a cell, the target nucleic acid comprises a sequence that is not found in the genome of the cell.

Suitable DNA/RNA binding conditions include physiological conditions normally present in a cell. Other suitable DNA/RNA binding conditions (e.g., conditions in a cell-free system) are known in the art: sec, e.g., Sambrook, supra. The strand of the target nucleic acid that is complementary to and hybridizes with the RNA guide is referred to as the “complementary strand” and the strand of the target nucleic acid that is complementary to the “complementary strand” (and is therefore not complementary to the RNA guide) is referred to as the “noncomplementary strand” or “non-complementary strand”.

### Preparation

In some embodiments, the variant polypeptide of the present invention can be prepared by (a) culturing bacteria which produce the variant polypeptide of the present invention, isolating the variant polypeptide, optionally, purifying the variant polypeptide, and complexing the variant polypeptide with RNA guide. The variant polypeptide can be also prepared by (b) a known genetic engineering technique, specifically, by isolating a gene encoding the variant polypeptide of the present invention from bacteria, constructing a recombinant expression vector, and then transferring the vector into an appropriate host cell that expresses the RNA guide for expression of a recombinant protein that complexes with the RNA guide in the host cell. Alternatively, the variant polypeptide can be prepared by (c) an in vitro coupled transcription-translation system and then complexes with RNA guide. Bacteria that can be used for preparation of the variant polypeptide of the present invention are not particularly limited as long as they can produce the variant polypeptide of the present invention. Some nonlimiting examples of the bacteria include E, coli cells described herein.

### Vectors

The present invention provides a vector for expressing the variant polypeptide described herein or nucleic acids encoding the variant described herein may be incorporated into a vector. In some embodiments, a vector of the invention includes a nucleotide sequence encoding variant polypeptide. In some embodiments, a vector of the invention includes a nucleotide sequence encoding the variant polypeptide.

The present invention also provides a vector that may be used for preparation of the variant polypeptide or compositions comprising the variant polypeptide as described herein. In some embodiments, the invention includes the composition or vector described herein in a cell. In some embodiments, the invention includes a method of expressing the composition comprising the variant polypeptide, or vector or nucleic acid encoding the variant polypeptide, in a cell. The method may comprise the steps of providing the composition, e.g., vector or nucleic acid, and delivering the composition to the cell.

Expression of natural or synthetic polynucleotides is typically achieved by operably linking a polynucleotide encoding the gene of interest, e.g., nucleotide sequence encoding the variant polypeptide, to a promoter and incorporating the construct into an expression vector. The expression vector is not particularly limited as long as it includes a polynucleotide encoding the variant polypeptide of the present invention and can be suitable for replication and integration in eukaryotic cells.

Typical expression vectors include transcription and translation terminators, initiation sequences, and promoters useful for expression of the desired polynucleotide. For example, plasmid vectors carrying a recognition sequence for RNA polymerase (pSP64, pBluescript, etc.), may be used. Vectors including those derived from retroviruses such as lentivirus are suitable tools to achieve long-term gene transfer since they allow long-term, stable integration of a transgene and its propagation in daughter cells. Examples of vectors include expression vectors, replication vectors, probe generation vectors, and sequencing vectors. The expression vector may be provided to a cell in the form of a viral vector.

Viral vector technology is well known in the art and described in a variety of virology and molecular biology manuals. Viruses which are useful as vectors include, but are not limited to phage viruses, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers.

The kind of the vector is not particularly limited, and a vector that can be expressed in host cells can be appropriately selected. To be more specific, depending on the kind of the host cell, a promoter sequence to ensure the expression of the variant polypeptide from the polynucleotide is appropriately selected, and this promoter sequence and the polynucleotide are inserted into any of various plasmids etc. for preparation of the expression vector.

Additional promoter elements, e.g., enhancing sequences, regulate the frequency of transcriptional initiation. Typically, these are located in the region 30-110 bp upstream of the start site, although a number of promoters have recently been shown to contain functional elements downstream of the start site as well.

Depending on the promoter, it appears that individual elements can function either cooperatively or independently to activate transcription.

Further, the disclosure should not be limited to the use of constitutive promoters. Inducible promoters are also contemplated as part of the disclosure. The use of an inducible promoter provides a molecular switch capable of turning on expression of the polynucleotide sequence which it is operatively linked when such expression is desired or turning off the expression when expression is not desired. Examples of inducible promoters include, but are not limited to a metallothionine promoter, a glucocorticoid promoter, a progesterone promoter, and a tetracycline promoter.

The expression vector to be introduced can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate transcriptional control sequences to enable expression in the host cells. Examples of such a marker include a dihydrofolate reductase gene and a neomycin resistance gene for eukaryotic cell culture: and a tetracycline resistance gene and an ampicillin resistance gene for culture of E, coli and other bacteria. By use of such a selection marker, it can be confirmed whether the polynucleotide encoding the variant polypeptide of the present invention has been transferred into the host cells and then expressed without fail.

The preparation method for recombinant expression vectors is not particularly limited, and examples thereof include methods using a plasmid, a phage or a cosmid.

### Methods of Expression

The present invention includes a method for protein expression, comprising translating the variant polypeptide described herein.

In some embodiments, a host cell described herein is used to express the variant polypeptide. The host cell is not particularly limited, and various known cells can be preferably used. Specific examples of the host cell include bacteria such as E, coli, yeasts (budding yeast, Saccharomyces cerevisiae, and fission yeast, Schizosaccharomyces pombe), nematodes (Caenorhabditis elegans), Xenopus laevis oocytes, and animal cells (for example, CHO cells, COS cells and HEK293 cells). The method for transferring the expression vector described above into host cells, i.e., the transformation method, is not particularly limited, and known methods such as electroporation, the calcium phosphate method, the liposome method and the DEAE dextran method can be used.

After a host is transformed with the expression vector, the host cells may be cultured, cultivated or bred, for production of the variant polypeptide. After expression of the variant polypeptide, the host cells can be collected and variant polypeptide purified from the cultures etc. according to conventional methods (for example, filtration, centrifugation, cell disruption, gel filtration chromatography, ion exchange chromatography, etc.).

In some embodiments, the methods for variant polypeptide expression comprises translation of at least 5 amino acids, at least 10 amino acids, at least 15 amino acids, at least 20 amino acids, at least 50 amino acids, at least 100 amino acids, at least 150 amino acids, at least 200 amino acids, at least 250 amino acids, at least 300 amino acids, at least 400 amino acids, at least 500 amino acids, at least 600 amino acids, at least 700 amino acids, at least 800 amino acids, at least 900 amino acids, or at least 1000 amino acids of the variant polypeptide. In some embodiments, the methods for protein expression comprises translation of about 5 amino acids, about 10 amino acids, about 15 amino acids, about 20 amino acids, about 50 amino acids, about 100 amino acids, about 150 amino acids, about 200 amino acids, about 250 amino acids, about 300 amino acids, about 400 amino acids, about 500 amino acids, about 600 amino acids, about 700 amino acids, about 800 amino acids, about 900 amino acids, about 1000 amino acids or more of the variant polypeptide.

A variety of methods can be used to determine the level of production of a mature variant polypeptide in a host cell. Such methods include, but are not limited to, for example, methods that utilize either polyclonal or monoclonal antibodies specific for the variant polypeptide or a labeling tag as described elsewhere herein. Exemplary methods include, but are not limited to, enzyme-linked immunosorbent assays (ELISA), radioimmunoassays (MA), fluorescent immunoassays (FIA), and fluorescent activated cell sorting (FACS). These and other assays are well known in the art (See, e.g., Maddox et al., J. Exp. Med. 158:1211 [1983]).

The present disclosure provides methods of in vivo expression of the variant polypeptide in a cell, comprising providing a polyribonucleotide encoding the variant polypeptide to a host cell wherein the polyribonucleotide encodes the variant polypeptide, expressing the variant polypeptide in the cell, and obtaining the variant polypeptide from the cell.

### Introduction of Alteration or Mutation

Nucleic acid sequences encoding variant polypeptides or variant polypeptides may be generated by synthetic methods known in the art. Using the nucleic acid sequence encoding the parent polypeptide itself as a framework, alternations or mutations can be inserted one or more at a time to alter the nucleic acid sequence encoding the parent polypeptide. Along the same lines, the parent polypeptide may be altered or mutated by introducing the changes into the polypeptide sequence as it is synthetically synthesized. This may be accomplished by methods well known in the art.

The production and introduction of alteration or mutation into a parent polypeptide sequence can be accomplished using any methods known by those of skill in the art. In particular, in some embodiments, oligonucleotide primers for PCR may be used for the rapid synthesis of a DNA template including the one or more alterations or mutations in the nucleic acid sequence encoding for the variant polypeptide. Site-specific mutagenesis may also be used as a technique useful in the preparation of individual peptides, or biologically functional equivalent proteins or peptides, through specific mutagenesis of the underlying DNA. The technique further provides a ready ability to prepare and test variants, incorporating one or more of the foregoing considerations, by introducing one or more nucleotide sequence changes into the DNA. Site-specific mutagenesis allows the production of variants through the use of specific oligonucleotide sequences which encode the DNA sequence of the desired mutation, as well as a sufficient number of adjacent nucleotides, to provide a primer sequence of sufficient size and sequence complexity to form a stable duplex on both sides of the deletion junction being traversed. Typically, a primer of about 17 to 25 nucleotides in length is preferred, with about 5 to 10 residues on both sides of the junction of the sequence being altered.

Introduction of structural variations, such as fusion of polypeptides as amino- and/or carboxyl-terminal extensions can be accomplished in a similar fashion as introduction of alterations or mutations into the parent polypeptide. The additional peptides may be added to the parent polypeptide or variant polypeptide by including the appropriate nucleic acid sequence encoding the additional peptides to the nucleic acid sequence encoding the parent polypeptide or variant polypeptide. Optionally, the additional peptides may be appended directly to the variant polypeptide through synthetic polypeptide production, 20)

In an aspect, the invention also provides methods for introducing an alteration or mutation into the parent polypeptide sequence to produce a variant polypeptide that has increased on-target binding with two or more loci (e.g., 2, 3, 4, 5, 6, 7, 8, 9, or more) of a target nucleic acid, as compared to a parent polypeptide.

In an aspect, the invention also provides methods for introducing an alteration or mutation into the parent polypeptide sequence to produce a plurality of variant polypeptides (e.g., separate variant polypeptides having the same amino acid sequence), that when individually complexed with a plurality of distinct RNA guides, have increased on-target binding with two or more loci of a target nucleic acid, as compared to a plurality of parent polypeptides and RNA guides.

In an aspect, the invention also provides methods for introducing an alteration or mutation into the parent polypeptide sequence to produce a variant polypeptide that has increased on-target ternary complex formation with two or more target loci of a target nucleic acid, as compared to a parent polypeptide.

In an aspect, the invention also provides methods for introducing an alteration or mutation into the parent polypeptide sequence to produce a plurality of variant polypeptides (e.g., separate variant polypeptides having the same amino acid sequence), that when individually complexed with a plurality of distinct RNA guides, have increased ternary complex formation with two or more loci of a target nucleic acid, as compared to a plurality of parent polypeptides and RNA guides.

In an aspect, the invention also provides methods for introducing an alteration or mutation into the parent polypeptide sequence to produce a variant polypeptide that exhibits targeting of an increased number of target nucleic acids or target loci, as compared to a parent polypeptide.

In an aspect, the invention also provides methods for introducing an alteration or mutation into the parent polypeptide sequence to produce a plurality of variant polypeptides (e.g., separate variant polypeptides having the same amino acid sequence), that when individually complexed with a plurality of distinct RNA guides, exhibit targeting of an increased number of target nucleic acids or target loci, as compared to a plurality of parent polypeptides and RNA guides.

In an aspect, the invention also provides methods for introducing an alteration or mutation into the parent polypeptide sequence to enhance stability of the variant polypeptide. Stability of the variant polypeptide can be determined by or may include a technique not limited to thermal denaturation assays, thermal shift assays, differential scanning calorimetry (DSC), differential scanning fluorimetry (DSF), isothermal titration calorimetry (ITC), pulse-chase methods, bleach-chase methods, cycloheximide-chase methods, circular dichroism (CD) spectroscopy, crystallization, and fluorescence-based activity assays.

### Variant Binary Complexing

Generally, the variant polypeptide and the RNA guide bind to each other in a molar ratio of about 1:1 to form the variant binary complex. The variant polypeptide and the RNA guide, either alone or together, do not naturally occur.

In some embodiments, the variant polypeptide can be overexpressed in a host cell and purified as described herein, then complexed with the RNA guide (e.g., in a test tube) to form a variant ribonucleoprotein (RNP) (e.g., variant binary complex).

In some embodiments, the variant binary complex exhibits increased binding affinity to a target nucleic acid, increased on-target binding activity, increased on-target binding specificity, increased ternary complex formation with a target nucleic acid, and/or increased stability over a range of incubation times. In some embodiments, the variant binary complex exhibits decreased off-target binding to a non-target nucleic acid and/or decreased dissociation from a target nucleic acid over a range of incubation times. In some embodiments, the variant binary complex exhibits increased target nucleic acid complex formation, target nucleic acid activity, and/or target nucleic acid specificity over a range of incubation times.

In some embodiments, complexation of a binary complex occurs at a temperature lower than about any one of 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 50° C., or 55° C. In some embodiments, the variant polypeptide does not dissociate from the RNA guide or bind to a free RNA at about 37° C. over an incubation period of at least about any one of 10 mins, 15 mins, 20 mins, 25 mins, 30 mins, 35 mins, 40 mins, 45 mins, 50 mins, 55 mins, Ihr, 2 hr, 3 hr, 4 hr, or more hours. In some embodiments, after binary complex formation, the variant ribonucleoprotein complex does not exchange the RNA guide with a different RNA.

In some embodiments, the variant polypeptide and RNA guide are complexed in a binary complexation buffer. In some embodiments, the variant polypeptide is stored in a buffer that is replaced with a binary complexation buffer to form a complex with the RNA guide. In some embodiments, the variant polypeptide is stored in a binary complexation buffer.

In some embodiments, the binary complexation buffer has a pH in a range of about 7.3 to 8.6 (e.g., about 7.5 to about 8.0, about 7.8 to 8.3, or about 8.0 to 8.6). In one embodiment, the pH of the binary complexation buffer is about 7.3. In one embodiment, the pH of the binary complexation buffer is about 7.4. In one embodiment, the pH of the binary complexation buffer is about 7.5. In one embodiment, the pH of the binary complexation buffer is about 7.6. In one embodiment, the pH of the binary complexation buffer is about 7.7. In one embodiment, the pH of the binary complexation buffer is about 7.8. In one embodiment, the pH of the binary complexation buffer is about 7.9. In one embodiment, the pH of the binary complexation buffer is about 8.0. In one embodiment, the pH of the binary complexation buffer is about 8.1. In one embodiment, the pH of the binary complexation buffer is about 8.2. In one embodiment, the pH of the binary complexation buffer is about 8.3. In one embodiment, the pH of the binary complexation buffer is about 8.4. In one embodiment, the pH of the binary complexation buffer is about 8.5. In one embodiment, the pH of the binary complexation buffer is about 8.6.

The thermostability of the variant polypeptide can increase under favorable conditions such as the addition of an RNA guide, e.g., binding an RNA guide.

In some embodiments, the variant polypeptide can be overexpressed and complexed with the RNA guide in a host cell prior to purification as described herein. In some embodiments, mRNA or DNA encoding the variant polypeptide is introduced into a cell so that the variant polypeptide is expressed in the cell. The RNA guide, which guides the variant polypeptide to the desired target nucleic acid is also introduced into the cell, whether simultaneously, separately or sequentially from a single mRNA or DNA construct, such that the necessary ribonucleoprotein complex is formed in the cell.

### Assessing Variant Binary Complex Stability and Functionality

Provided herein in certain embodiments are methods for identifying an optimal variant polypeptide/RNA guide complex (referred to herein as the variant binary complex) including (a) combining a variant polypeptide and an RNA guide in a sample to form the variant binary complex: (b) measuring a value of the variant binary complex: and (c) determining the variant binary complex is optimal over the reference molecule, if the value of the variant binary complex is greater than a value of a reference molecule. In some embodiments, the value may include, but is not limited to, a stability measurement (e.g., Tₘ value, thermostability), a rate of binary complex formation, RNA guide binding specificity, and/or complex activity.

In some embodiments, an optimal variant polypeptide/RNA guide complex (i.e., a variant binary complex) is identified by the steps of: (a) combining a variant polypeptide and an RNA guide in a sample to form the variant binary complex: (b) detecting a Tₘ value of the variant binary complex: and (c) determining the variant binary complex is stable if the Tₘ value of the variant binary complex is greater than a Tₘ value of a reference molecule or a Tₘ reference value by at least 8° C.

The methods involving a step of measuring the thermostability of a variant polypeptide/RNA guide complex (i.e., a variant binary complex) may include, without limitation, methods of determining the stability of a variant binary complex, methods of determining a condition that promotes a stable variant binary complex, methods of screening for a stable variant binary complex, and methods for identifying an optimal gRNA to form a stable variant binary complex. In certain embodiments, a thermostability value of a variant binary complex may be measured.

Additionally, in certain embodiments, a thermostability value of a reference molecule may also be measured. In certain embodiments, a variant binary complex may be determined to be stable if the measured thermostability value of the variant binary complex is greater than the measured thermostability value of the reference molecule or a thermostability reference value, measured under the same experimental conditions, as described herein. In certain embodiments, the reference molecule may be the variant polypeptide absent an RNA guide.

20)

In certain embodiments, the thermostability value that is measured may be a denaturation temperature value. In these embodiments, the thermostability reference value is a denaturation temperature reference value. In certain embodiments, the thermostability value that is measured may be a Tₘ value. In these embodiments, the thermostability reference value may be a Tₘ reference value. In certain embodiments, the thermostability value may be measured using a thermal shift assay. In certain embodiments, an assay used to measure thermostability may involve a technique described herein including, but not limited to, thermal denaturation assays, thermal shift assays, differential scanning calorimetry (DSC), differential scanning fluorimetry (DSF), isothermal titration calorimetry (ITC), pulse-chase methods, bleach-chase methods, cycloheximide-chase methods, circular dichroism (CD) spectroscopy, crystallization, and fluorescence-based activity assays.

In certain embodiments, a variant binary complex may be identified if the rate of variant polypeptide/RNA guide complex formation, RNA guide binding specificity, and/or complex activity of the variant binary complex is greater than a value of the reference molecule or the reference value (e.g., a value of a parent polypeptide/RNA guide complex, referred to herein as a parent binary complex). For example, in certain embodiments, the variant binary complex may be identified if the value of a rate of variant polypeptide/RNA guide complex formation, RNA guide binding specificity, and/or complex activity of the variant binary complex is at least X % greater than a value of the reference molecule or the reference value (e.g., a value of a parent binary complex). In certain embodiments, the methods described herein may further comprise steps that include measuring the activity of the variant binary complex as described herein.

### Variant Ternary Complexing

In some embodiments, the variant polypeptide, RNA guide, and target nucleic acid, as described herein, form a variant ternary complex (e.g., in a test tube or cell). Generally, the variant polypeptide, the RNA guide, and the target nucleic acid associate with each other in a molar ratio of about 1:1:1 to form the variant ternary complex. The variant polypeptide, the RNA guide, and the target nucleic acid, either alone or together, do not naturally occur.

In some embodiments, the variant binary complex (e.g., complex of variant polypeptide and RNA guide) as described herein, is further complexed with the target nucleic acid (e.g., in a test tube or cell) to form a variant ternary complex.

In some embodiments, complexation of the ternary complex occurs at a temperature lower than about any one of 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 50° C., or 55° C. In some embodiments, the variant binary complex does not dissociate from the target nucleic acid or bind to a free nucleic acid (e.g., free DNA) at about 37° C. over an incubation period of at least about any one of 10 mins, 15 mins, 20 mins, 25 mins, 30 mins, 35 mins, 40 mins, 45 mins, 50 mins, 55 mins, Ihr, 2 hr, 3 hr, 4 hr, or more hours. In some embodiments, after ternary complex formation, a variant binary complex does not exchange the target nucleic acid with a different nucleic acid.

In some embodiments, the variant polypeptide, RNA guide, and target nucleic acid are complexed in a ternary complexation buffer. In some embodiments, the variant polypeptide is stored in a buffer that is replaced with a ternary complexation buffer to form a complex with the RNA guide and target nucleic acid. In some embodiments, the variant polypeptide is stored in a ternary complexation buffer.

In some embodiments, the variant binary complex and target nucleic acid are complexed in a ternary complexation buffer. In some embodiments, the variant binary complex is stored in a buffer that is replaced with a ternary complexation buffer to form a complex with the target nucleic acid. In some embodiments, the variant binary complex is stored in a ternary complexation buffer.

In some embodiments, the ternary complexation buffer has a pH in a range of about 7.3 to 8.6 (e.g., about 7.5 to about 8.0, about 7.8 to 8.3, or about 8.0 to 8.6). In one embodiment, the pH of the ternary complexation buffer is about 7.3. In one embodiment, the pH of the ternary complexation buffer is about 7.4. In one embodiment, the pH of the ternary complexation buffer is about 7.5. In one embodiment, the pH of the ternary complexation buffer is about 7.6. In one embodiment, the pH of the ternary complexation buffer is about 7.7. In one embodiment, the pH of the ternary complexation buffer is about 7.8. In one embodiment, the pH of the ternary complexation buffer is about 7.9. In one embodiment, the pH of the ternary complexation buffer is about 8.0. In one embodiment, the pH of the ternary complexation buffer is about 8.1. In one embodiment, the pH of the ternary complexation buffer is about 8.2. In one embodiment, the pH of the ternary complexation buffer is about 8.3. In one embodiment, the pH of the ternary complexation buffer is about 8.4. In one embodiment, the pH of the ternary complexation buffer is about 8.5. In one embodiment, the pH of the ternary complexation buffer is about 8.6.

The thermostability of a variant polypeptide can increase under favorable conditions such as the addition of an RNA guide and target nucleic acid.

### Assessing Variant Ternary Complex Stability and Functionality

Provided herein in certain embodiments are methods for identifying an optimal variant ternary complex including (a) combining a variant polypeptide, an RNA guide, and a target nucleic acid in a sample to form the variant ternary complex: (b) measuring a value of the variant ternary complex: and (c) determining the variant ternary complex is optimal over the reference molecule, if the value of the variant ternary complex is greater than a value of a reference molecule. In some embodiments, the value may include, but is not limited to, a stability measurement (e.g., Tₘ value, thermostability), a rate of ternary complex formation, a DNA binding affinity measurement, a DNA binding specificity measurement, and/or a complex activity measurement (e.g., nuclease activity measurement).

In some embodiments, an optimal variant ternary complex is identified by the steps of: (a) combining a variant polypeptide, an RNA guide, and a target nucleic acid in a sample to form the variant ternary complex: (b) detecting a Tₘ value of the variant ternary complex: and (c) determining the variant ternary complex is stable if the Tₘ value of the variant ternary complex is greater than a Tₘ value of a reference molecule or a Tₘ reference value by at least 8° C.

The methods involving a step of measuring the thermostability of a variant ternary complex may include, without limitation, methods of determining the stability of a variant ternary complex, methods of determining a condition that promotes a stable variant ternary complex, methods of screening for a stable variant ternary complex, and methods for identifying an optimal binary complex to form a stable variant ternary complex. In certain embodiments, a thermostability value of a variant ternary complex may be measured.

Additionally, in certain embodiments, a thermostability value of a reference molecule may also be measured. In certain embodiments, a variant ternary complex may be determined to be stable if the measured thermostability value of the variant ternary complex is greater than the measured thermostability value of the reference molecule or a thermostability reference value, measured under the same experimental conditions, as described herein. In certain embodiments, the reference molecule may be the variant polypeptide absent an RNA guide and/or target nucleic acid.

In certain embodiments, the thermostability value that is measured may be a denaturation temperature value. In these embodiments, the thermostability reference value is a denaturation temperature reference value. In certain embodiments, the thermostability value that is measured may be a Tₘ value. In these embodiments, the thermostability reference value may be a Tₘ reference value. In certain embodiments, the thermostability value may be measured using a thermal shift assay. In certain embodiments, an assay used to measure thermostability may involve a technique described herein including, but not limited to, differential scanning fluorimetry (DSF), differential scanning calorimetry (DSC), or isothermal titration calorimetry (ITC).

In certain embodiments, a variant ternary complex may be identified if the rate of ternary complex formation, DNA binding affinity, DNA binding specificity, and/or complex activity (e.g., nuclease activity) of the variant ternary complex is greater than a value of the reference molecule or the reference value (e.g., a value of a parent ternary complex). For example, in certain embodiments, the variant ternary complex may be identified if the value of a rate of ternary complex formation, DNA binding affinity, DNA binding specificity, and/or complex activity of the variant ternary complex is at least X % greater than a value of the reference molecule or the reference value (e.g., a value of a parent ternary complex). In certain embodiments, the methods described herein may further comprise steps that include measuring the activity of the variant ternary complex as described herein.

### Delivery

Compositions or complexes described herein may be formulated, for example, including a carrier, such as a carrier and/or a polymeric carrier, e.g., a liposome, and delivered by known methods to a cell (e.g., a prokaryotic, eukaryotic, plant, mammalian, etc.). Such methods include, but not limited to, transfection (e.g., lipid-mediated, cationic polymers, calcium phosphate, dendrimers): electroporation or other methods of membrane disruption (e.g., nucleofection), viral delivery (e.g., lentivirus, retrovirus, adenovirus, AAV), microinjection, microprojectile bombardment (“gene gun”), fugene, direct sonic loading, cell squeezing, optical transfection, protoplast fusion, impalefection, magnetofection, exosome-mediated transfer, lipid nanoparticle-mediated transfer, and any combination thereof.

In some embodiments, the method comprises delivering one or more nucleic acids (e.g., nucleic acids encoding the variant polypeptide, RNA guide, donor DNA, etc.), one or more transcripts thereof, and/or a pre-formed variant polypeptide/RNA guide complex (i.e., variant binary complex) to a cell. Exemplary intracellular delivery methods, include, but are not limited to: viruses or virus-like agents: chemical-based transfection methods, such as those using calcium phosphate, dendrimers, liposomes, or cationic polymers (e.g., DEAE-dextran or polyethylenimine): non-chemical methods, such as microinjection, electroporation, cell squeezing, sonoporation, optical transfection, impalefection, protoplast fusion, bacterial conjugation, delivery of plasmids or transposons: particle-based methods, such as using a gene gun, magnectofection or magnet assisted transfection, particle bombardment: and hybrid methods, such as nucleofection. In some embodiments, the present application further provides cells produced by such methods, and organisms (such as animals, plants, or fungi) comprising or produced from such cells.

### Cells

Polypeptides, compositions or complexes described herein may be delivered to a variety of cells. In some embodiments, the cell is an isolated cell. In some embodiments the cell is in cell culture. In some embodiments, the cell is ex vivo. In some embodiments, the cell is obtained from a living organism, and maintained in a cell culture. In some embodiments, the cell is a single-cellular organism.

In some embodiments, the cell is a prokaryotic cell. In some embodiments, the cell is a bacterial cell or derived from a bacterial cell. In some embodiments, the bacterial cell is not related to the bacterial species from which the parent polypeptide is derived. In some embodiments, the cell is an archacal cell or derived from an archacal cell. In some embodiments, the cell is a eukaryotic cell. In some embodiments, the cell is a plant cell or derived from a plant cell. In some embodiments, the cell is a fungal cell or derived from a fungal cell. In some embodiments, the cell is an animal cell or derived from an animal cell. In some embodiments, the cell is an invertebrate cell or derived from an invertebrate cell. In some embodiments, the cell is a vertebrate cell or derived from a vertebrate cell. In some embodiments, the cell is a mammalian cell or derived from a mammalian cell. In some embodiments, the cell is a human cell. In some embodiments, the cell is a zebra fish cell. In some embodiments, the cell is a rodent cell. In some embodiments, the cell is synthetically made, sometimes termed an artificial cell.

In some embodiments, the cell is derived from a cell line. A wide variety of cell lines for tissue culture are known in the art. Examples of cell lines include, but are not limited to, 293T, MF7, K562, HeLa. and transgenic varieties thereof. Cell lines are available from a variety of sources known to those with skill in the art (see, e.g., the American Type Culture Collection (ATCC) (Manassas, Va.)). In some embodiments, a cell transfected with one or more nucleic acids (such as Ago-coding vector and gDNA) or Ago-gDNA complex described herein is used to establish a new cell line comprising one or more vector-derived sequences to establish a new cell line comprising modification to the target nucleic acid. In some embodiments, cells transiently or non-transiently transfected with one or more nucleic acids (such as variant polypeptide-encoding vector and RNA guide) or variant polypeptide/RNA guide complex (i.e., variant binary complex) described herein, or cell lines derived from such cells are used in assessing one or more test compounds.

In some embodiments, the method comprises introducing into a host cell one or more nucleic acids comprising nucleotide sequences encoding a DNA-targeting RNA (e.g., RNA guide) and/or the variant polypeptide. In one embodiment, a cell comprising a target DNA is in vitro, in vivo, or ex vivo. In other embodiments, nucleic acids comprising nucleotide sequences encoding a DNA-targeting RNA (e.g., RNA guide) and/or the variant polypeptide include recombinant expression vectors e.g., including but not limited to adeno-associated virus constructs, recombinant adenoviral constructs, recombinant lentiviral constructs, recombinant retroviral constructs, and the like.

In some embodiments, the cell is a primary cell. For example, cultures of primary cells can be passaged 0 times, 1 time, 2 times, 4 times, 5 times, 10 times, 15 times or more. In some embodiments, the primary cells are harvest from an individual by any known method. For example, leukocytes may be harvested by apheresis, leukocytapheresis, density gradient separation, etc. Cells from tissues such as skin, muscle, bone marrow, spleen, liver, pancreas, lung, intestine, stomach, etc. can be harvested by biopsy. An appropriate solution may be used for dispersion or suspension of the harvested cells. Such solution can generally be a balanced salt solution. (e.g., normal saline, phosphate-buffered saline (PBS), Hank's balanced salt solution, etc.), conveniently supplemented with fetal calf serum or other naturally occurring factors, in conjunction with an acceptable buffer at low concentration. Buffers can include HEPES, phosphate buffers, lactate buffers, etc. Cells may be used immediately, or they may be stored (e.g., by freezing). Frozen cells can be thawed and can be capable of being reused. Cells can be frozen in a DMSO, serum, medium buffer (e.g., 10% DMSO, 50% scrum, 40% buffered medium), and/or some other such common solution used to preserve cells at freezing temperatures.

In some embodiments, the variant polypeptide has nuclease activity that induces double-stranded breaks or single-stranded breaks in a target nucleic acid, (e.g., genomic DNA). The double-stranded break can stimulate cellular endogenous DNA-repair pathways, including Homology Directed Recombination (HDR), Non-Homologous End Joining (NHEJ), or Alternative Non-Homologues End-Joining (A-NHEJ). NHEJ can repair cleaved target nucleic acid without the need for a homologous template. This can result in deletion or insertion of one or more nucleotides into the target nucleic acid. HDR can occur with a homologous template, such as the donor DNA. The homologous template can comprise sequences that are homologous to sequences flanking the target nucleic acid cleavage site. In some cases, HDR can insert an exogenous polynucleotide sequence into the cleaved target nucleic acid. The modifications of the target DNA due to NHEJ and/or HDR can lead to, for example, mutations, deletions, alterations, integrations, gene correction, gene replacement, gene tagging, transgene knock-in, gene disruption, and/or gene knock-outs.

In some embodiments, the cell culture is synchronized to enhance the efficiency of the methods. In some embodiments, cells in S and G2 phases are used for HDR-mediated gene editing. In some embodiments, the cell can be subjected to the method at any cell cycle. In some embodiments, cell over-plating significantly reduces the efficacy of the method. In some embodiments, the method is applied to a cell culture at no more than about any one of 40%, 45%, 50%, 55%, 60%, 65%, or 70% confluency.

In some embodiments, binding of the variant polypeptide/RNA guide complex (i.e., variant binary complex) to the target nucleic acid in the cell recruits one or more endogenous cellular molecules or pathways other than DNA repair pathways to modify the target nucleic acid. In some embodiments, binding of the variant binary complex blocks access of one or more endogenous cellular molecules or pathways to the target nucleic acid, thereby modifying the target nucleic acid. For example, binding of the variant binary complex may block endogenous transcription or translation machinery to decrease the expression of the target nucleic acid.

In some embodiments, a method for modifying a target DNA molecule in a cell is provided. The method comprises contacting the target DNA molecule inside of a cell with a variant polypeptide described herein: and a single molecule DNA-targeting RNA comprising, in 5′ to 3′ order, a first nucleotide segment that hybridizes with a target sequence of the target DNA molecule: a nucleotide linker; and a second nucleotide segment that hybridizes with the first nucleotide segment to form a double-stranded RNA duplex. The variant polypeptide forms a complex with the single molecule DNA-targeting RNA inside the cell and the target DNA molecule is modified.

### Kits

The invention also provides kits that can be used, for example, to carry out a method described herein. In some embodiments, the kits include a variant polypeptide of the invention, e.g., a variant of Table 2. In some embodiments, the kits include a polynucleotide that encodes such a variant polypeptide, and optionally the polynucleotide is comprised within a vector, e.g., as described herein. The kits also can optionally include an RNA guide, e.g., as described herein. The RNA guide of the kits of the invention can be designed to target a sequence of interest, as is known in the art. The CRISPR nuclease variant and the RNA guide can be packaged within the same vial or other vessel within a kit or can be packaged in separate vials or other vessels, the contents of which can be mixed prior to use. The kits can additionally include, optionally, a buffer and/or instructions for use of the CRISPR nuclease variant and/or RNA guide.

All references and publications cited herein are hereby incorporated by reference.

### EXAMPLES

The following examples are provided to further illustrate some embodiments of the present invention but are not intended to limit the scope of the invention: it will be understood by their exemplary nature that other procedures, methodologies, or techniques known to those skilled in the art may alternatively be used.

### Example 1—Engineering of Variant Constructs

In this Example, variant constructs were generated.

DNA templates comprising single mutations were constructed via two PCR steps using mutagenic forward and mutagenic reverse primers ordered from IDT™ (Integrated DNA Technologies, Inc.). In the first step, two sets of PCR reactions were conducted in 384 plates to generate two fragments. The overlapping regions of two PCR fragments contained the desired single mutations and allowed the assembly of the entire DNA template via a second PCR. In the second step, the purified fragments from the first step were used as the template for the overlapping PCR (OL PCR) and the Fw and Rv oligos annealing to the vector backbone as the OL PCR primers. The resulting linear DNA templates contained a T7 promoter, a T7 terminator, and the open-reading frame for the polypeptide.

These linear DNA templates were used directly in a cell-free transcription and translation system to express the polypeptide variants containing the single mutations. The variant constructs were further individually transferred into transient transfection vectors. Additionally, DNA templates comprising combinatorial mutations were prepared by PCR and subsequently transferred into transient transfection vectors.

### Example 2—Florescence Polarization Assay for Variant Binary Complex Detection

In this Example, the ability of a wild-type or variant nuclease polypeptide and an RNA guide to form a binary complex is assessed through a fluorescence polarization assay.

Linear ssDNA fragments comprising the reverse complement of the T7 RNA polymerase promoter sequence upstream of the direct repeat sequence and desired 20 bp RNA guide target are synthesized by IDT™. Linear dsDNA in vitro transcription (IVT) templates are then generated by annealing a universal T7 forward oligo (95-4° C. at 5° C./minute) to the reverse complement ssDNA and filled in with Klenow fragment (New England Biolabs R) for 15 minutes at 25° C. The resulting IVT template is then transcribed into an RNA guide using the HiScribe T7 High Yield RNA Synthesis Kit (New England Biolabs R) at 37° C. for 4 hours. Following transcription, each RNA guide is purified using an RNA Clean and Concentrator Kit (Zymo) and stored at −20° C. until use.

The RNA guide is then labeled with 6-carboxyfluorescein (6-FAM) (IDT™), 25 nM nuclease polypeptide (wild-type or variant polypeptide) in 1× assay buffer (20 mM Tris-HCl (pH 7.5), 150 mM KCl. 5 mM MgCl₂, 1 mM DTT) is titrated with increasing concentrations of labeled RNA guide (7.5-250 nM). Complexes are incubated at 37° C. for 30 minutes before taking fluorescence polarization measurements using a microplate reader (Infinite R 200 Pro. Tecan).

Binary complex formation at different temperatures is also investigated. Further binding experiments as described above are performed isothermally at 25, 50, 60, and 70° C.

Formation of a binary complex upon titration of a nuclease polypeptide (wild-type or variant polypeptide) with increasing concentrations of RNA guide (or formation of a binary complex upon titration of RNA guide with increasing concentrations of a nuclease polypeptide) results in changes in fluorescence polarization signal, in millipolarization (mP) units. A binding curve is generated by plotting changes in fluorescence polarization signal over a range of RNA guide concentrations.

This Example indicates how binding affinities of nuclease polypeptides (wild-type or variant polypeptide) to RNA guides can be determined and compared.

### Example 3—RNA Electrophoretic Mobility Shift Assay for Variant Binary Complex Detection

This Example describes use of an RNA EMSA to determine the ability of a nuclease polypeptide (wild-type or variant) to bind to an RNA guide.

Synthetic RNA guides from IDT™ are labeled with a 5′ IRDye.R, 800CW (also referred to as IR800 dye or IR800) using 5′ EndTag Labeling Kit (Vector R. Laboratories) and IRDye.R, 800CW Maleimide (LI-COR® Biosciences), as previously detailed in Yan et al., 2018. After labeling, the RNA guides are cleaned and concentrated via phenol chloroform extraction. Concentrations are quantified by Nanodrop™.

For RNA binding assays, nuclease polypeptides (wild-type or variant polypeptides) are diluted to 2.5 μM in 1× binding buffer (50 mM NaCl, 10 mM Tris-HCl, 10 mM MgCl₂, 1 mM DTT, pH 7.9. Polypeptides are then serially diluted from 2.5 μM to 37.5 μM in 1× binding buffer. The polypeptides are again diluted 1:10 in 1× binding buffer plus 50 nM IR800 labeled RNA guide and mixed thoroughly. These reactions can further include 0.5-5 μg tRNA, which serves as a competitive inhibitor to decrease nonspecific binding of polypeptide to RNA and thereby facilitate accurate specific binding determinations. Reactions are incubated at 37° C. for 1 hour, 1 μL 100× bromophenol blue is added to the reactions for dye front visualization, then the entire reaction is loaded onto a 6% DNA Retardation Gel (ThermoFisher Scientific™), which runs for 90 minutes at 80V. The gel is imaged on the Licor® Odyssey® CLx.

This assay relies on the principle that the rate at which RNA migrates through the gel is determined by its size. An RNA only sample is able to migrate a particular distance. However, if the RNA binds to a polypeptide, a band that represents a larger, less mobile RNA complex appears, which is “upshifted” on the gel.

Therefore, the intensities of two bands are measured: 1) an RNA only band and 2) a polypeptide-bound “upshifted” RNA band. If all RNA is bound to a polypeptide, only an upshifted band is observed. As the concentration of polypeptide decreases, the intensity of the upshifted band decreases, while the intensity of the RNA only band increases. In comparing RNA binding affinities for nuclease polypeptides (wild-type or variant polypeptides), a higher polypeptide/RNA affinity is characterized by more specific binding at lower concentrations of polypeptide.

This Example indicates how binding affinities of wild-type nuclease polypeptides to RNA guides and binding affinities of variant polypeptides to RNA guides can be determined and compared.

### Example 4—In Vitro Cleavage Assay for Variant Binary Complexes

This Example describes methods for preparing RNPs and for determining in vitro biochemical activity of the RNPs.

Vectors encoding a wild-type or variant polypeptide are transformed into E, coli BL21 (DE3) (New England Biolabs R) and expressed under a T7 promoter. Transformed cells are initially grown overnight in 5 mL Luria Broth (TEKNOVA™)+50 μg/mL kanamycin, followed by inoculation into 1 L Terrific Broth media (TEKNOVA™)+50 μg/mL kanamycin. Cells are grown at 37° C. until an OD600 of 0.6-0.8, then protein expression is induced with 0.5 mM IPTG. Cultures are then grown at 18° C. for an additional 14-18 hours. Cultures are harvested and pelleted via centrifugation, then resuspended in 1 mL extraction buffer per 5 g cell pellet (50 mM HEPES, pH 7.5, 500 mM NaCl, 5% glycerol, 0.5 mM TCEP). Cells are lysed via cell disruptor (Constant System Limited), then centrifuged at 20,000×g for 20 minutes at 4° C. in order to clarify the lysate, 0.2% polyethylenimine (PEI) is added to the clarified lysate and incubated at 4° C. with constant end-over-end rotation for 20 minutes. The lysate is then centrifuged again at 20,000×g for 10 minutes. The lysate is purified via ion exchange chromatography. After purification, fractions are run on SDS-PAGE gels, and fractions containing protein of the appropriate size are pooled and concentrated using 30 kD Amicon Ultra15 Centrifugal Units. Proteins are buffer exchanged into 12.5 mM HEPES pH 7.0, 120 mM NaCl, 0.5 mM TCEP, and 50% glycerol. Concentrations are then measured using the Nanodrop (ThermoFisher Scientific™), and proteins are stored at −20° C.

RNPs are prepared using a 2:1 ratio of synthetic crRNA (Integrated DNA Technologies) to protein. The RNPs are complexed for 30 minutes at 37° C. in 1× NEBuffer™ 2 (NEB2: New England Biolabs®; 50 mM NaCl, 10 mM Tris-HCl, 10 mM MgCl₂, 1 mM DTT, pH 7.9). After complexing, the RNPs are diluted using 1× NEB2 as a dilution buffer. Apo reactions (protein without RNA guide) are prepared in the same manner, making up the volume of crRNA with H₂O.

A target dsDNA substrate (Integrated DNA Technologies) is added at 20 nM to the RNP and apo samples. Reactions are mixed thoroughly then incubated at 37° C. for 1 hour, then quenched with 1 μL 20 mg/mL Proteinase K (ThermoFisher Scientific™). Reactions were incubated for another 15 minutes at 50° C., then the entire reaction was run on a 2% agarose E-gel (ThermoFisher Scientific™). Gels were visualized by ethidium bromide on a Gel Doc™ EZ Gel Imager (BioRad R).

The intensities of two types of bands are measured: 1) a full-length (uncleaved) DNA band and 2) one or more downshifted cleaved DNA bands. An inactive RNP is characterized by a full-length DNA band. An active RNP yields one or more downshifted cleaved DNA bands. As the concentration of an active RNP decreases, the intensity of the full-length band increases, and the intensity of the cleaved band(s) decreases. In comparing activity of multiple RNPs, an RNP having higher activity than another is characterized by more intense cleaved bands at lower RNP concentrations.

The method of this Example allows for the comparison of in vitro cleavage activity of wild-type or variant RNPs (binary complexes) on target DNA.

### Example 5—In Vitro Stability Assays of Variant Polypeptides and Variant Binary Complexes

In this Example, the stability of a variant RNP is assessed.

For the accelerated stability study, RNPs (5 μM) are generated in the same manner as described in Example 4, and the samples are subsequently stored at 25° C. for 48 hours.

In vitro cleavage assays (as described in Example 4) are performed on the RNP samples. These results are compared with those of Example 4 to determine the extent to which variant RNPs stored at 25° C. for 48 hours retain biochemical activity.

Apo polypeptide (without RNA guide) is also incubated at 25° C. for 48 hours. RNA EMSA assays are performed on the apo samples using the method described in Example 3. These results are compared with those of Example 3 to determine the extent to which a variant polypeptide is able to form a binary complex with an RNA guide.

Apo samples incubated at 25° C. for 48 hours are also complexed with RNA guides to form RNPs, using the method described in Example 4. In vitro cleavage assays are then performed according to the methods of Example 4. The assay results are compared with those of Example 4 to assess activity levels of variant RNPs formed with protein incubated at 25ºC.

30 The methods of this Example allow for comparison of the stability of wild-type and variant polypeptides and wild-type and variant RNPs (binary complexes). An nuclease polypeptide demonstrating greater specific binding to an RNA guide than another nuclease polypeptide to the RNA guide is indicative of a more stable polypeptide. An RNP demonstrating more robust in vitro cleavage of a target DNA than cleavage by another RNP is indicative of a more stable binary complex.

### Example 6—DNA Electrophoretic Mobility Shift Assay for Variant Ternary Complex Detection

This Example describes use of a DNA EMSA to determine the ability of an RNA guide, a nuclease polypeptide (wild-type or variant polypeptide), and a target DNA substrate to form a ternary complex.

Vectors encoding a wild-type or variant polypeptide are transformed into E, coli BL21 (DE3) (New England BioLabs®) and BL21(DE3)pLySS (Novagen®). Transformed cells are initially grown overnight in 5 mL Luria Broth (TEKNOVA™)+50 μg/mL kanamycin, followed by inoculation into 1 L Terrific Broth media (TEKNOVA™)+50 μg/mL kanamycin. Cells are grown at 37° C. until an OD600 of 0.6-0.8, then protein expression is induced with 0.5 mM IPTG. Cultures are then grown at 18° C. for an additional 14-18 hours. Cultures are harvested and pelleted via centrifugation, then resuspended in 1 mL extraction buffer per 5 g cell pellet (50 mM HEPES, pH 7.5. 500 mM NaCl, 5% glycerol, 0.5 mM TCEP). Cells are lysed via cell disruptor (Constant System Limited), then centrifuged at 20,000×g for 20 minutes at 4° C. in order to clarify the lysate, 0.2% polyethylenimine (PEI) is added to the clarified lysate and incubated at 4° C. with constant end-over-end rotation for 20 minutes. The lysate is then centrifuged again at 20.000×g for 10 minutes. The lysate is purified via ion exchange chromatography. After purification, fractions are run on SDS-PAGE gels, and fractions containing protein of the appropriate size are pooled and concentrated using 30 kD Amicon R. Ultra15 Centrifugal Units. Proteins were buffer exchanged into 12.5 mM HEPES pH 7.0. 120 mM NaCl, 0.5 mM TCEP, and 50% glycerol. Concentrations were then measured using the Nanodrop™ (ThermoFisher Scientific™) and proteins were stored at −20° C.

RNPs are prepared using a 2:1 ratio of synthetic RNA guide (Integrated DNA Technologies. IDT™) to polypeptide. Targets adjacent to the PAM sequences disclosed herein are selected, and RNA guides are designed using a direct repeat sequence as described herein. The RNPs are complexed for 30 minutes at 37° C. in 1× NEBuffer™ (NEB2: New England Biolabs R: 50 mM NaCl, 10 mM Tris-HCl, 10 mM MgCl₂. I mM DTT, pH 7.9). After complexing, a 5 point 1:2 serial dilution from 5 μM to 37.5 μM is performed using 1× NEB2 as a dilution buffer. Apo reactions (polypeptide without RNA guide) are prepared in the same manner, making up the volume of RNA guide with H₂O.

dsDNA target substrates are generated by PCR from an oligo (Integrated DNA Technologies). Before PCR, the 5′ end of the forward primer is labeled an IR800 dye, as described in Yan et al., 2018. Using Amplitaq Gold R (ThermoFisher Scientific™), the dsDNA substrate is then amplified with the IR800 labeled forward primer and unlabeled reverse primer. The resulting dsDNA is purified with a DNA Clean and Concentrator Kit (Zymo) and quantified by Nanodrop™ (ThermoFisher Scientific™).

RNP samples and Apo (control) samples are diluted 1:10 into 1× binding buffer (50 mM NaCl, 10 mM Tris-HCl, 1 mM TCEP, 10% glycerol, 2 mM EDTA, pH 8.0) plus 20 nM IR800 labeled target DNA substrate and mixed thoroughly. Reactions are incubated at 37° C. for 1 hour. Bromophenol blue is added to the reactions for dye front visualization, then the entire reaction is loaded onto a 6% DNA Retardation Gel (ThermoFisher Scientific™), which ran for 90 minutes at 80V. The gel is imaged on the Licor® Odyssey® CLx.

In this assay, the rate at which DNA migrates through the gel is determined by its size. A DNA only sample is able to migrate a particular distance. However, if an RNP binds to the DNA, a band that represents a larger, less mobile DNA complex appears, which is “upshifted” on the gel.

This Example shows how the affinity of variant RNPs (variant binary complexes) to DNA targets (to produce a ternary complex) can be compared to the affinity of wild-type RNPs (wild-type binary complexes to the DNA targets.

### Example 7—Targeting of Mammalian Genes by Combination Variant Polypeptides

This Example describes indel assessment on multiple targets using wild-type and variant effectors (e.g., CRISPR nuclease variants) introduced into mammalian cells by transient transfection.

The wild-type effector of SEQ ID NO: 3 (WT) and CRISPR nuclease variants of SEQ ID NO: 3 were individually cloned into a pcda3.1 backbone (Invitrogen). The plasmids were then maxi-prepped and diluted. Targets adjacent to the PAM sequences disclosed herein were selected, and RNA guides were designed using a direct repeat sequence as described herein. The RNA guide and target sequences are shown in Table 5. RNA guides were cloned into a pUC19 backbone (New England Biolabs R). The plasmids were then maxi-prepped and diluted.

TABLE 5
Mammalian targets and corresponding crRNAs.

| Target | PAM | | |
|---|---|---|---|
| identifier | sequence | Target sequence | crRNA sequence |
| EMX1 | 5′-ATTG-3′ | CCGCCGCTTCCTGAGCCA | CUUGUUGUAUAUGUCCUUUUAUA |
| | | TC (SEQ ID NO: 16) | GGUAUUAAACAACCCGCCGCUUC |
| | | | CUGAGCCAUC (SEQ ID NO: 17) |
| VEGFA | 5′-TTTA-3′ | TCCAGACCACCAATGGGC | CUUGUUGUAUAUGUCCUUUUAUA |
| | | AC (SEQ ID NO: 18) | GGUAUUAAACAACUCCAGACCAC |
| | | | CAAUGGGCAC (SEQ ID NO: 19) |
| AAVS1 | 5′-TTTG-3′ | TGAGAATGGTGCGTCCTA | CUUGUUGUAUAUGUCCUUUUAUA |
| | | GG (SEQ ID NO: 20) | GGUAUUAAACAACUGAGAAUGGU |
| | | | GCGUCCUAGG (SEQ ID NO: 28) |

Approximately 16 hours prior to transfection, 25,000 HEK293T cells in DMEM/10% FBS+Pen/Strep (D10 media) were plated into each well of a 96-well plate. On the day of transfection, the cells were 70-90% confluent. For each well to be transfected, a mixture of Lipofectamine 2000™ and Opti-MEM™ was prepared and incubated at room temperature for 5 minutes (Solution 1). After incubation, the Lipofectamine 2000™:Opti-MEM™ mixture was added to a separate mixture containing nuclease plasmid, RNA guide plasmid, and Opti-MEM™ (Solution 2). In the case of negative controls, the RNA guide plasmid was not included in Solution 2. Solutions 1 and 2 were mixed by pipetting up and down, then incubated at room temperature for 25 minutes. Following incubation, the Solution 1 and 2 mixture was added dropwise to each well of a 96-well plate containing the cells. Approximately 72 hours post transfection, cells were trypsinized by adding TrypLE™ to the center of each well and incubating at 37° C. for approximately 5 minutes. D10 media was then added to each well and mixed to resuspend cells. The resuspended cells were centrifuged at 500 g for 10 minutes to obtain a pellet, and the supernatant was discarded. The cell pellet was then resuspended in QuickExtract™ buffer (Lucigen®), and cells were incubated at 65° C. for 15 minutes, 68° C. for 15 minutes, and 98° C. for 10 minutes.

Samples for Next Generation Sequencing were prepared by two rounds of PCR. The first round (PCR1) was used to amplify specific genomic regions depending on the target. Round 2 PCR (PCR2) was performed to add Illumina adapters and indices. Reactions were then pooled and purified by column purification. Sequencing runs were performed using a 150 Cycle NextSeq 500/550 Mid or High Output v2.5 Kit.

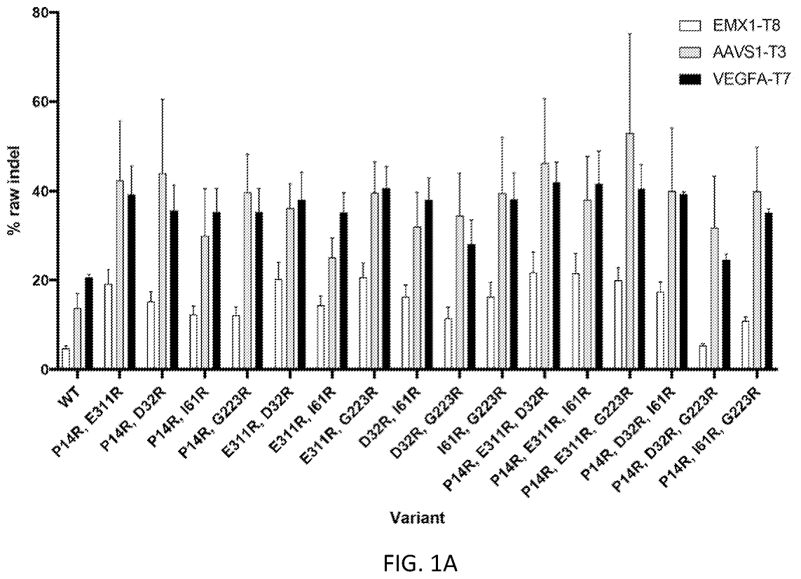

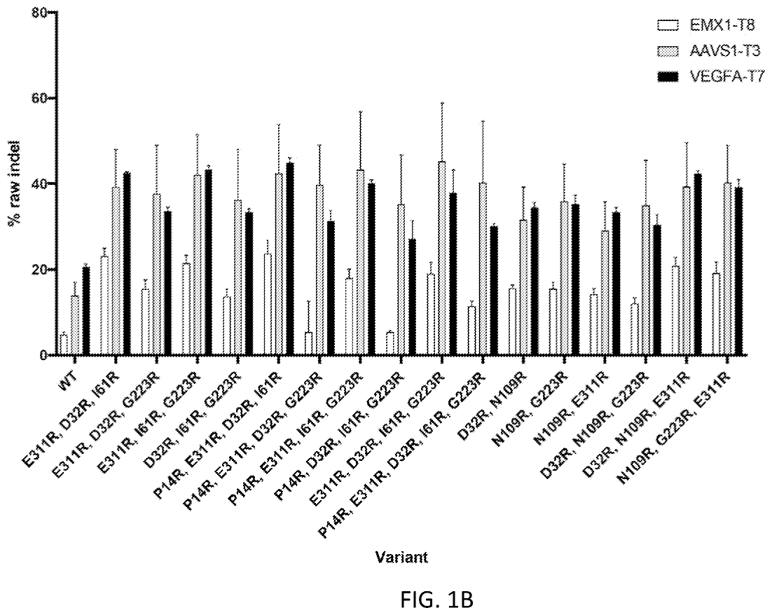

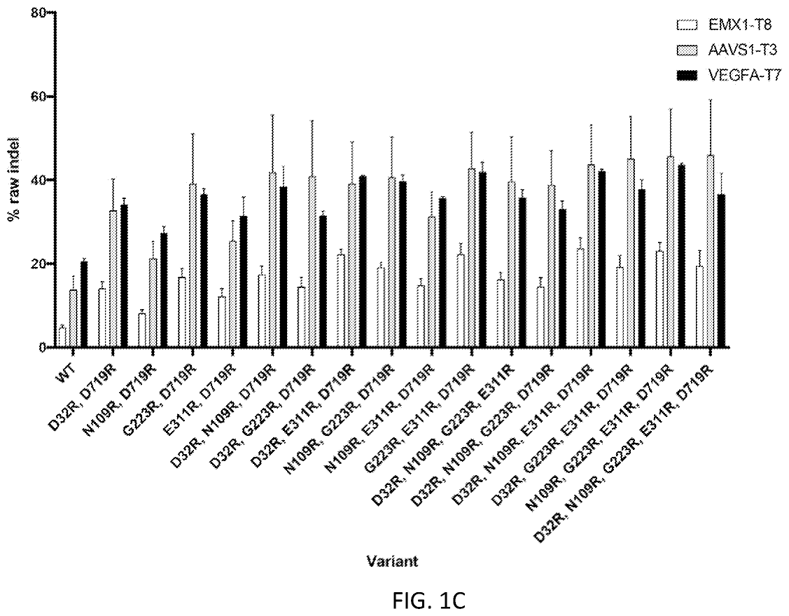

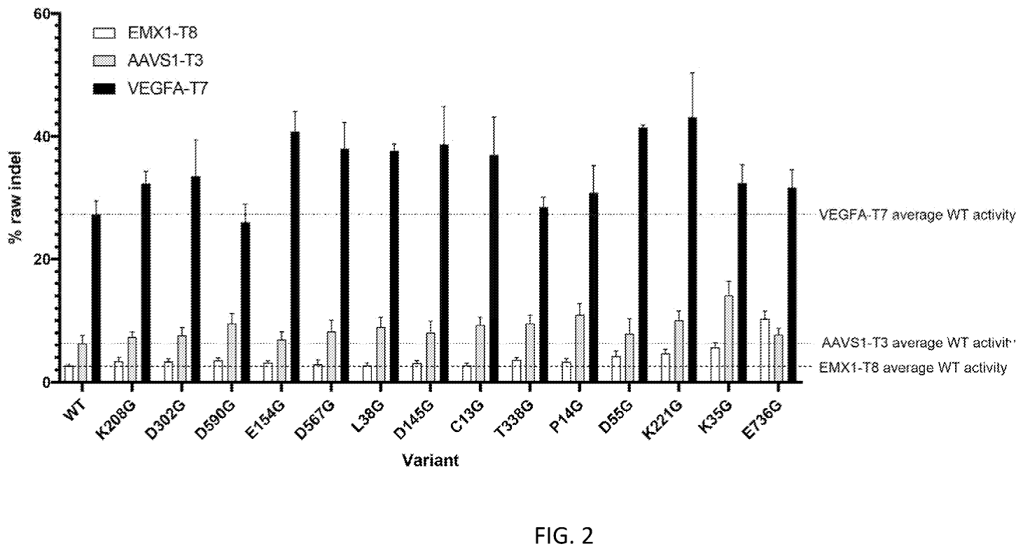

Of the first thirty-one variants engineered, each consisting of a single arginine substitution relative to the parent polypeptide of SEQ ID NO: 3, twenty-six of the variants demonstrated increased indel activity compared to the parent polypeptide. Seven of the top-performing point mutants were further screened in combinations of two, three, four, and five following the same method described above. The results in FIGS. 1-3 demonstrate that substitutions P14R. E311R. D32R, 161R. G223R. N109R, and/or D719R increased nuclease activity compared to the parent polypeptide of SEQ ID NO: 3.

The variant polypeptide comprising P14R. E311R, and D32R substitutions: the variant polypeptide comprising P14R. E311R, and G223R substitutions: the variant polypeptide comprising P14R. E311R. D32R, and I61R substitutions; and the variant polypeptide comprising D32R. N109R. E311R, and D719R substitutions were further tested for indel activity at an additional three AAVSI, three VEGFA, and three EMX1 targets. Indel activity for each of the variant polypeptides averaged across the twelve targets was approximately 6—to 7-fold higher compared to the indel activity of the parent polypeptide. The variant comprising P14R. E311R. D32R, and 161R substitutions yielded the greatest increase in indel activity of the four variants tested on the twelve-target set.

### Example 8—Targeting of Mammalian Genes by Point Variant Polypeptides

This Example describes indel assessment on multiple targets using wild-type and variant effectors introduced into mammalian cells by transient transfection. Seventy-nine variants, each comprising a single glycine substitution relative to SEQ ID NO: 3, were individually cloned into a pcDNA3.1 backbone (Invitrogen), as described in Example 7. The RNA guides and cell transfection protocol were as described in Example 7.

The following fourteen variants exhibited increased indel activity at all targets compared to the parent polypeptide of SEQ ID NO: 3: K208G, D302G, D590G, E154G, D567G, L38G, D145G, C13G, T338G, P14G, D55G, K221G, K35G, and E736G. The indel activity for the variant polypeptides is shown in FIG. 4. Indel activity for each of the variants averaged across the three targets tested was approximately 1.2—to 2.1-fold higher compared to the indel activity of the parent polypeptide. Each of the fourteen variants exhibited increased indel activity at the three targets, with the exception of D590G, which performed similarly to the parent polypeptide at the VEGFA target. E736G yielded the greatest increase in indel activity of all variants tested.

What is claimed is:

1. A variant polypeptide comprising an alteration relative to a parent polypeptide of SEQ ID NO: 3, and wherein the alteration is a substitution of Table 2.

2. The variant polypeptide of claim 1, wherein the substitution is a P14R substitution, an E31IR substitution, a D32R substitution, an I61R substitution, a G223R substitution, an N109R substitution, and/or a D719R substitution.

3. The variant polypeptide of claim 1 or 2, wherein the variant polypeptide comprises a) a P14R substitution, an E311R substitution, and a D32R substitution; b) a P14R substitution, an E311R substitution, and a G223R substitution; c) a P14R substitution, an E311R substitution, a D32R substitution, and an I61R substitution; or d) a D32R substitution, an N109R substitution, an E311R substitution, and a D719R substitution.

4. The variant polypeptide of any one of claims 1 to 3, wherein the variant polypeptide further comprises a K208G substitution, a D302G substitution, a D590G substitution, an E154G substitution, a D567G substitution, an L38G substitution, a D145G substitution, a C13G substitution, a T338G substitution, a P14G substitution, a D55G substitution, a K221G substitution, a K35G substitution, and an E736G substitution.

5. A variant polypeptide comprising an amino acid sequence having at least 95% identity to SEQ ID NO: 3 and comprising a substitution one or more of positions P14, E311, D32, 161, G223, N109, and D719 relative to SEQ ID NO: 3.

6. A variant polypeptide comprising an amino acid sequence having at least 95% identity to SEQ ID NO: 3 and comprising one or more of the following substitutions: P14R, E311R, D32R, 161R, G223R, N109R, and D719R.

7. A variant polypeptide comprising an amino acid sequence having at least 95% identity to SEQ ID NO: 3 and comprising a substitution one or more of positions K208, D302, D590, E154, D567, L38, D145, C13, T338, P14, D55, K221, K35, and E736 relative to SEQ ID NO: 3.

8. A variant polypeptide comprising an amino acid sequence having at least 95% identity to SEQ ID NO: 3 and comprising one or more of the following substitutions: K208G, D302G, D590G, E154G, D567G, L38G, D145G, C13G, T338G, P14G, D55G, K221G, K35G, and E736G.

9. The variant polypeptide of any one of claims 1-8, wherein the variant polypeptide comprises a substitution at P14 (e.g., a P14R substitution),

10. The variant polypeptide of any one of claims 1-9, wherein the variant polypeptide comprises a substitution at E311 (e.g., an E311R substitution),

11. The variant polypeptide of any one of claims 1-10, wherein the variant polypeptide comprises a substitution at D32 (e.g., a D32R substitution),

12. The variant polypeptide of any one of claims 1-11, wherein the variant polypeptide comprises a substitution at 161 (e.g., a I61R substitution),

13. The variant polypeptide of any one of claims 1-12, wherein the variant polypeptide comprises a substitution at G223 (e.g., a G223 substitution),

14. The variant polypeptide of any one of claims 1-13, wherein the variant polypeptide comprises a substitution at N109 (e.g., a N109R substitution),

15. The variant polypeptide of any one of claims 1-14, wherein the variant polypeptide comprises a substitution at D719 (e.g., a D719R substitution),

16. The variant polypeptide of claim 5, wherein the variant polypeptide comprises a substitution at position P14 (e.g., a P14R substitution), an E311 (e.g., an E31IR substitution), a D32 (e.g., a D32R substitution), an 161 (e.g., a I61R substitution), a G223 (e.g., a G223 substitution), an N109 (e.g., a N109R substitution), a D719 (e.g., a D719R substitution), or any combination thereof.

17. The variant polypeptide of claim 16, wherein the variant polypeptide comprises a substitution at position P14 (e.g., a P14R substitution), E311 (e.g., an E311R), and D32 (e.g., a D32R substitution) relative to SEQ ID NO: 3 (e.g., a P14R, E311R, D32R variant polypeptide).

18. The variant polypeptide of claim 16, wherein the variant polypeptide comprises a substitution at position P14 (e.g., a P14R substitution), E311 (e.g., an E311R), and G223 (e.g., a G223R substitution) relative to SEQ ID NO: 3 (e.g., a P14R, E311R, G223R variant polypeptide).

19. The variant polypeptide of claim 16, wherein the variant polypeptide comprises a substitution at position P14 (e.g., a P14R substitution), E311 (e.g., an E311R), D32 (e.g., a D32R substitution), and I61 (e.g., an I61R substitution) relative to SEQ ID NO: 3 (e.g., a P14R, E311R, D32R and I61R variant polypeptide).

20. The variant polypeptide of claim 16, wherein the variant polypeptide comprises a substitution at position D32R (e.g., a D32R substitution), N109 (e.g., an N109R), E311 (e.g., an E311R substitution), and D719 (e.g., a D719R substitution) relative to SEQ ID NO: 3 (e.g., a D32R, N109R, E311R and D719R variant polypeptide).

21. The variant polypeptide of any one of claims 5-20, wherein the variant polypeptide comprises a substitution at K208 (e.g., a K208G substitution), D302 (e.g., a D302G substitution), D590 (e.g., a D590G substitution), E154 (e.g., an E154G substitution), D567 (e.g., a D567G substitution), L38 (e.g., an L38G substitution), D145 (e.g., a D145G substitution), C13 (e.g., a C13G substitution), T338 (e.g., a T338G substitution), P14 (e.g., a P14G substitution), D55 (e.g., a D55G substitution), K221 (e.g., a K221G substitution), K35 (e.g., a K35G substitution), and E736 (e.g., an E736G substitution), or any combination thereof.

22. The variant polypeptide of any one of claims 1-21, wherein the variant polypeptide exhibits increased binary complex formation with an RNA guide, relative to a parent polypeptide.

23. The variant polypeptide of any one of claims 1-22, wherein a binary complex comprising the variant polypeptide exhibits increased stability, relative to a parent binary complex.

24. The variant polypeptide of any one of claims 1-23, wherein the variant polypeptide exhibits increased nuclease activity, relative to a parent polypeptide.

25. A composition comprising the variant polypeptide of any one of claims 1-24, wherein the composition further comprises an RNA guide or a nucleic acid encoding the RNA guide, wherein the RNA guide comprises a direct repeat sequence and a spacer sequence.

26. The composition of claim 25, wherein the direct repeat sequence is at least 90% identical to any one of SEQ ID NOs: 4-13 or comprises a sequence having at least 90% identity to SEQ ID NO: 14 or SEQ ID NO: 15.

27. The composition of claim 25, wherein the direct repeat sequence is at least 95% identical to any one of SEQ ID NOs: 4-13 or comprises a sequence having at least 95% identity to SEQ ID NO: 14 or SEQ ID NO: 15.

28. The composition of claim 25, wherein the direct repeat sequence is any one of SEQ ID NOs: 4-13 or comprises a sequence of SEQ ID NO: 14 or SEQ ID NO: 15.

29. The composition of any one of claims 25-28, wherein the spacer sequence comprises about 15 nucleotides to about 35 nucleotides in length.

30. The composition of any one of claims 25-29, wherein the spacer sequence binds to a target strand sequence of a target nucleic acid, and wherein a non-target strand sequence of the target nucleic acid sequence is adjacent to a protospacer adjacent motif (PAM) sequence.

31. The composition of claim 30, wherein the PAM sequence is 5′-TTR-3′, 5′-NTTR-3′, 5′-NTTN-3′, 5′-RTTR-3′, 5′-ATTR-3′, or 5′-RTTG-3′, wherein N is any nucleotide, Y is C or T, and R is A or G.

32. The composition of claim 31, wherein the PAM sequence is 5′-TTG-3′, 5′-TTA-3′, 5′-ATTG-3′, 5′-TTTA-3′, or 5′-TTTG-3′.

33. The variant polypeptide or the composition of any previous claim, wherein the variant polypeptide further comprises a nuclear localization signal (NLS).

34. The variant polypeptide or the composition of any previous claim, wherein the variant polypeptide further comprises a peptide tag, a fluorescent protein, a base-editing domain, a DNA methylation domain, a histone residue modification domain, a localization factor, a transcription modification factor, a light-gated control factor, a chemically inducible factor, or a chromatin visualization factor.

35. A composition comprising a nucleic acid that encodes the variant polypeptide and/or the RNA guide of any previous claim.

36. The composition of claim 35, wherein the nucleic acid is codon-optimized for expression in a cell.

37. The composition of claim 35 or 36, wherein the nucleic acid is operably linked to a promoter.

38. The composition of any one of claims 35-37, wherein the nucleic acid is in a vector.

39. The composition of claim 38, wherein the vector comprises a retroviral vector, a lentiviral vector, a phage vector, an adenoviral vector, an adeno-associated vector, or a herpes simplex vector.

40. The variant polypeptide or the composition of any previous claim, wherein the variant polypeptide is present in a delivery system comprising a nanoparticle (e.g., a lipid nanoparticle), a liposome, an exosome, a microvesicle, or a gene-gun.

41. A cell comprising the variant polypeptide or the composition of any previous claim.

42. The cell of claim 41, wherein the cell is a eukaryotic cell.

43. The cell of claim 41 or 42, wherein the cell is a mammalian cell or a plant cell.

44. The cell of any one of claims 41-43, wherein the cell is a human cell.

45. A composition comprising a variant polypeptide or a complex comprising the variant polypeptide, wherein the variant polypeptide comprises an alteration relative to a parent polypeptide of SEQ ID NO: 3, and wherein the variant polypeptide or the complex exhibits enhanced enzymatic activity, enhanced binding activity, enhanced binding specificity, and/or enhanced stability, relative to a parent polypeptide or a complex comprising the parent polypeptide.

46. The composition of claim 45, wherein the alteration is a substitution of Table 2.

47. The composition of claim 46, wherein the substitution is a P14R substitution, an E311R substitution, a D32R substitution, an I61R substitution, a G223R substitution, an N109R substitution, and/or a D719R substitution.

48. The composition of claim 46 or 47, wherein the variant polypeptide comprises a) a P14R substitution, an E311R substitution, and a D32R substitution; b) a P14R substitution, an E311R substitution, and a G223R substitution; c) a P14R substitution, an E31IR substitution, a D32R substitution, and an 161R substitution: or d) a D32R substitution, an N109R substitution, an E311R substitution, and a D719R substitution.

49. The composition of claim 47 or 48, wherein the variant polypeptide further comprises a K208G substitution, a D302G substitution, a D590G substitution, an E154G substitution, a D567G substitution, an L38G substitution, a D145G substitution, a C13G substitution, a T338G substitution, a P14G substitution, a D55G substitution, a K221G substitution, a K35G substitution, and an E736G substitution.

50. The composition of any one of claims 45-49, wherein the enhanced enzymatic activity is enhanced nuclease activity.

51. The composition of any one of claims 45-50, wherein the variant polypeptide exhibits enhanced binding activity to an RNA guide, relative to the parent polypeptide.

52. The composition of any one of claims 45-51, wherein the variant polypeptide exhibits enhanced binding specificity to an RNA guide, relative to the parent polypeptide.

53. The composition of any one of claims 45-52, wherein the complex comprising the variant polypeptide is a variant binary complex that further comprises an RNA guide, and the variant binary complex exhibits enhanced binding activity to a target nucleic acid (e.g., on-target binding activity), relative to a parent binary complex.

54. The composition of any one of claims 45-53, wherein the complex comprising the variant polypeptide is a variant binary complex that further comprises an RNA guide, and the variant binary complex exhibits enhanced binding specificity to a target nucleic acid (e.g., on-target binding specificity), relative to a parent binary complex.

55. The composition of any one of claims 45-54, wherein the complex comprising the variant polypeptide is a variant binary complex that further comprises an RNA guide, and the variant binary complex exhibits enhanced stability, relative to a parent binary complex.

56. The composition of any one of claims 45-55, wherein the variant binary complex and a target nucleic acid form a variant ternary complex, and the variant ternary complex exhibits increased stability, relative to a parent ternary complex.

57. The composition of any one of claims 45-56, wherein the variant polypeptide further exhibits enhanced binary complex formation, enhanced protein-RNA interactions, and/or decreased dissociation from an RNA guide, relative to the parent polypeptide.

58. The composition of any one of claims 45-57, wherein the variant binary complex further exhibits decreased dissociation from a target nucleic acid, and/or decreased off-target binding to a non-target nucleic acid, relative to the parent binary complex.

59. The composition of any one of claims 45-58, wherein the enhanced enzymatic activity, enhanced binding activity, enhanced binding specificity, and/or enhanced stability occur over a range of temperatures, e.g., 20° C. to 65° C.

60. The composition of any one of claims 45-59, wherein the enhanced enzymatic activity, enhanced binding activity, enhanced binding specificity, and/or enhanced stability occur over a range of incubation times.

61. The composition of any one of claims 45-60, wherein the enhanced enzymatic activity, enhanced binding activity, enhanced binding specificity, and/or enhanced stability occur in a buffer having a pH in a range of about 7.3 to about 8.6.

62. The composition of any one of claims 45-61, wherein the enhanced enzymatic activity, enhanced binding activity, enhanced binding specificity, and/or enhanced stability occurs when a Tₘ value of the variant polypeptide, variant binary complex, or variant ternary complex is at least 8° C. greater than the Tₘ value of the parent polypeptide, parent binary complex, or parent ternary complex.

63. The composition of any one of claims 45-62, wherein the variant polypeptide comprises a RuvC domain or a split RuvC domain.

64. The composition any one of claims 45-63, wherein the parent polypeptide comprises the sequence of SEQ ID NO: 3.

65. The composition of any one of claims 45-64, wherein the RNA guide comprises a direct repeat sequence and a spacer sequence.

66. The composition of claim 65, wherein the direct repeat sequence is at least 90% identical to any one of SEQ ID NOs: 4-13 or comprises a sequence having at least 90% identity to SEQ ID NO: 14 or SEQ ID NO: 15.

67. The composition of claim 65, wherein the direct repeat sequence is at least 95% identical to any one of SEQ ID NOs: 4-13 or comprises a sequence having at least 95% identity to SEQ ID NO: 14 or SEQ ID NO: 15.

68. The composition of claim 65, wherein the direct repeat sequence is any one of SEQ ID NOs: 4-13 or comprises a sequence of SEQ ID NO: 14 or SEQ ID NO: 15.

69. The composition of any one of claims 65-68, wherein the spacer sequence comprises between 15 and 35 nucleotides in length.

70. The composition of any one of claims 65-69, wherein the spacer sequence comprises complementarity to a target strand sequence of a target nucleic acid.

71. The composition of claim 70, wherein the target nucleic acid comprises a non-target strand sequence adjacent to a protospacer adjacent motif (PAM) sequence.

72. The composition of claim 71, wherein the PAM sequence is 5′-TTR-3′, 5′-NTTR-3′, 5′-NTTN-3′, 5′-RTTR-3′, 5′-ATTR-3′, or 5′-RTTG-3′, wherein N is any nucleotide, Y is C or T, and R is A or G.

73. The composition of claim 72, wherein the PAM sequence is 5′-TTG-3′, 5′-TTA-3′, 5′-ATTG-3′, 5′-TTTA-3′, or 5′-TTTG-3′.

74. The composition of any one of claims 45-73, wherein the variant polypeptide further comprises a peptide tag, a fluorescent protein, a base-editing domain, a DNA methylation domain, a histone residue modification domain, a localization factor, a transcription modification factor, a light-gated control factor, a chemically inducible factor, or a chromatin visualization factor.

75. A composition comprising a nucleic acid that encodes the variant polypeptide of any one of claims 45-74, wherein optionally the nucleic acid is codon-optimized for expression in a cell.

76. The composition of claim 75, wherein the cell is a eukaryotic cell.

77. The composition of claim 75 or 76, wherein the cell is a mammalian cell or a plant cell.

78. The composition of any one of claims 75-77, wherein the cell is a human cell.

79. The composition of any one of claims 75-78, wherein the nucleic acid encoding the variant polypeptide is operably linked to a promoter.

80. The composition of any one of claims 75-79, wherein the nucleic acid encoding the variant polypeptide is in a vector.

81. The composition of claim 80, wherein the vector comprises a retroviral vector, a lentiviral vector, a phage vector, an adenoviral vector, an adeno-associated vector, or a herpes simplex vector.

82. The composition of any one of claims 45-81, wherein the composition is present in a delivery composition comprising a nanoparticle (e.g., a lipid nanoparticle), a liposome, an exosome, a microvesicle, or a gene-gun.

83. A method for editing a gene in a cell, the method comprising contacting the cell with the variant polypeptide or composition of any one of the previous claims.

84. A nucleic acid molecule encoding a variant polypeptide of any of claims 1-83.

85. The nucleic acid molecule of claim 84, wherein the sequence of the nucleic acid molecule is 95% identical to a sequence selected from the group consisting of SEQ ID NOs: 1, 2, or 21-27.

86. The nucleic acid molecule of claim 84, wherein the sequence of the nucleic acid molecule comprises a sequence selected from the group consisting of SEQ ID NOs: 1, 2, or 21-27.

87. The nucleic acid molecule of claim 84, wherein the sequence of the nucleic acid molecule is 95% identical to a sequence selected from the group consisting of SEQ ID NOs: 22, 23, or 25.


SEQUENCE LISTING (28 sequences)

SEQ ID NO: 1 - DNA, 2235 bp, Unknown
  Description of Unknown parent sequence
atgatcaagtctattcagctgaaggtcaagggggagtgtccgatcacgaaggatgtaatcaacgaatacaaagaatactataacaattgcagtgattggattaaaaacaatctgacgtccattaccatcggggagatggcaaaatttctgcaatcgttgagcgataaagaagtggcctatatctcaatgggcctgtccgatgagtggaaagacaaaccgttatatcatctgtttaccaaaaaatatcacaccaaaaatgcggataacttattatactactacattaaagaaaaaaatctggacggctacaaaggcaatacgcttaatatctccaatacatctttccgccagttcggttatttcaaactcgtggtgagcaactaccgcactaaaatccgtacgctgaattgtaaaatcaagcgtaagaaaatcgatgccgattccacgtctgaggatatcgaaatgcaggtgatgtacgaaattattaaatacagtttaaataaaaagtctgattgggataacttcattagctatatcgaaaacgttgaaaatcctaatattgacaacatcaaccgctacaaactgctgcgcgaatgcttttgcgaaaacgaaaacatgattaagaacaaacttgaattgctgtctgtggaacaattgaaaaaatttggcggttgcatcatgaaacctcacattaatagcatgaccattaacattcaagattttaaaattgaggaaaaagaaaactctctggggttcatcctccacctcccactgaacaaaaaacagtatcaaattgaactcctgggcaatcgtcagattaaaaaaggcaccaaagaaattcacgaaacgttagttgacattactaacacccatggcgaaaacattgtgtttactattaaaaatgataatctgtatatcgtgttctcttatgagtccgaatttgaaaaagaagaagttaacttcgctaaaacggttggcctggacgtaaacttcaaacatgccttctttgtgacctctgagaaagataactgtcatctcgacggttatattaatctctacaaatacttattggagcacgacgagtttactaaccttctcaccgaagatgaacgtaaagattatgaggagctgagtaaagtcgttactttctgcccgtttgaaaatcagttactgtttgcgcgttacaacaagatgagcaaattctgcaagaaagaacaggtcctgagcaaacttctctatgcgctgcagaaaaaactcaaagacgagaaccgcacgaaagaatatatttatgtctcgtgcgtgaacaaattacgtgccaaatatgtgtcatattttattctgaaagaaaagtactacgagaagcagaaagaatatgacatcgaaatgggctttgtggacgactcaacggaaagcaaagaatcaatggataaacgccgtactgaatatccgtttcgcaacacgccggtagccaacgaactgttgtccaaactgaataacgtacagcaggacatcaacgggtgcctgaagaacatcatcaactacatttataaaattttcgagcagaacggttataaagttgtcgccctcgaaaacctggaaaattctaattttgaaaaaaaacaggtgttgccgacgattaaaagtctgctgaaatatcacaaactggagaaccagaacgtgaatgatatcaaggcctctgacaaagttaaagaatatattgaaaacggttattatgaactcatgaccaacgagaataacgaaatcgttgatgcaaaatatacagaaaagggcgcaatgaaggtgaagaacgccaatttttttaacctcatgatgaaaagtttgcattttgccagtgtgaaagatgagtttgtgctgctgtccaataatggcaagacgcagattgcattagtgccatccgagtttacatctcagatggacagcaccgatcactgtctgtacatgaagaagaacgacaaaggtaaactggtgaaagcggataaaaaggaagttcgtacaaaacaggaacgtcacatcaacggcctcaacgccgatttcaacgcagcgaataatattaaatatatcgtggaaaatgaagtgtggcgtggtattttttgcactcgcccgaagaaaacagaatataacgtacccagtctggataccacgaaaaaaggtccgtctgcgattctcaacatgctgaagaaaattgaagccatcaaggtcctggaaacggaaaaa

SEQ ID NO: 2 - DNA, 2235 bp, Unknown
  Description of Unknown parent sequence
atgatcaagagcatccagctgaaggtgaagggcgagtgccccatcaccaaggacgtgatcaacgagtacaaggagtactacaacaactgttctgattggatcaagaacaatctgaccagcatcacaatcggcgagatggccaagtttctgcagagcctgtccgacaaggaggtggcctacatctctatgggcctgagcgacgagtggaaggataagcctctgtatcacctgttcaccaagaagtaccacacaaagaatgccgacaacctgctgtactattacatcaaggagaagaacctggatggctacaagggcaataccctgaacatctccaatacatctttcaggcagtttggctatttcaagctggtggtgtccaattacaggaccaagatccgcacactgaactgcaagatcaagcgcaagaagatcgacgccgattctaccagcgaggacatcgagatgcaggtcatgtatgagatcatcaagtactccctgaacaagaagtctgattgggataatttcatctcttatatcgagaacgtggagaaccccaatatcgataacatcaatcggtacaagctgctgagagagtgcttttgtgagaacgagaatatgatcaagaacaagctggagctgctgagcgtggagcagctgaagaagttcggcggctgtatcatgaagcctcacatcaacagcatgaccatcaatatccaggactttaagatcgaggagaaggagaattccctgggcttcatcctgcacctgccactgaacaagaagcagtaccagatcgagctgctgggcaatcggcagatcaagaagggcaccaaggagatccacgagacactggtggacatcaccaacacacacggcgagaacatcgtgtttacaatcaagaacgataatctgtacatcgtgtttagctacgagtccgagttcgagaaggaggaagtgaattttgccaagaccgtgggcctggacgtgaacttcaagcacgccttctttgtgacatccgagaaggacaattgccacctggatggctatatcaacctgtataagtacctgctggagcacgatgagttcaccaacctgctgacagaggacgagcggaaggattacgaggagctgtctaaggtggtgaccttttgccctttcgagaatcagctgctgtttgccagatataacaagatgagcaagttctgtaagaaggagcaggtgctgtccaagctgctgtacgccctgcagaagaagctgaaggacgagaacaggacaaaggagtatatctacgtgtcttgcgtgaataagctgcgcgccaagtatgtgagctactttatcctgaaggagaagtattacgagaagcagaaggagtatgacatcgagatgggcttcgtggacgattctaccgagagcaaggagtccatggataagcggagaaccgagtacccctttaggaacacacctgtggccaatgagctgctgagcaagctgaacaatgtgcagcaggatatcaacggctgtctgaagaacatcatcaattacatctacaagatcttcgagcagaacggctacaaggtggtggccctggagaacctggagaactccaatttcgagaagaagcaggtgctgccaacaatcaagtctctgctgaagtatcacaagctggagaaccagaatgtgaacgacatcaaggccagcgataaggtgaaggagtacatcgagaatggctattacgagctgatgaccaacgagaacaatgagatcgtggacgccaagtatacagagaagggcgccatgaaggtgaagaatgccaacttctttaacctgatgatgaagagcctgcactttgcctccgtgaaggatgagttcgtgctgctgtccaacaatggcaagacccagatcgccctggtgccatccgagttcacctctcagatggacagcacagatcactgcctgtacatgaagaagaatgacaagggcaagctggtgaaggccgataagaaggaggtgaggacaaagcaggagcgccacatcaacggcctgaatgccgactttaacgccgccaacaatatcaagtatatcgtggagaatgaagtgtggcggggcatcttctgtaccagaccaaagaagacagagtacaacgtgccctccctggataccacaaagaagggcccctctgccatcctgaatatgctgaagaagatcgaggccatcaaggtgctggagaccgagaag

SEQ ID NO: 3 - protein, 745 aa, Unknown
  Description of Unknown parent sequence
MIKSIQLKVKGECPITKDVINEYKEYYNNCSDWIKNNLTSITIGEMAKFLQSLSDKEVAYISMGLSDEWKDKPLYHLFTKKYHTKNADNLLYYYIKEKNLDGYKGNTLNISNTSFRQFGYFKLVVSNYRTKIRTLNCKIKRKKIDADSTSEDIEMQVMYEIIKYSLNKKSDWDNFISYIENVENPNIDNINRYKLLRECFCENENMIKNKLELLSVEQLKKFGGCIMKPHINSMTINIQDFKIEEKENSLGFILHLPLNKKQYQIELLGNRQIKKGTKEIHETLVDITNTHGENIVFTIKNDNLYIVFSYESEFEKEEVNFAKTVGLDVNFKHAFFVTSEKDNCHLDGYINLYKYLLEHDEFTNLLTEDERKDYEELSKVVTFCPFENQLLFARYNKMSKFCKKEQVLSKLLYALQKKLKDENRTKEYIYVSCVNKLRAKYVSYFILKEKYYEKQKEYDIEMGFVDDSTESKESMDKRRTEYPFRNTPVANELLSKLNNVQQDINGCLKNIINYIYKIFEQNGYKVVALENLENSNFEKKQVLPTIKSLLKYHKLENQNVNDIKASDKVKEYIENGYYELMTNENNEIVDAKYTEKGAMKVKNANFFNLMMKSLHFASVKDEFVLLSNNGKTQIALVPSEFTSQMDSTDHCLYMKKNDKGKLVKADKKEVRTKQERHINGLNADFNAANNIKYIVENEVWRGIFCTRPKKTEYNVPSLDTTKKGPSAILNMLKKIEAIKVLETEK

SEQ ID NO: 4 - RNA, 36 nt, Artificial Sequence
  Description of Artificial Sequence Synthetic oligonucleotide
cuuguuguauauguccuuuuauagguauuaaacaac

SEQ ID NO: 5 - RNA, 36 nt, Artificial Sequence
  Description of Artificial Sequence Synthetic oligonucleotide
cuuguuguauauacgcuuuuauagguauuaaacaac

SEQ ID NO: 6 - RNA, 36 nt, Artificial Sequence
  Description of Artificial Sequence Synthetic oligonucleotide
cuuguuguauauacgcuuuuauagguauugaacaac

SEQ ID NO: 7 - RNA, 36 nt, Artificial Sequence
  Description of Artificial Sequence Synthetic oligonucleotide
cuuguuguauauauccuuuuauagauauuaaacagc

SEQ ID NO: 8 - RNA, 36 nt, Artificial Sequence
  Description of Artificial Sequence Synthetic oligonucleotide
cuuguuguauauauccuuuuauagauguugaacaac

SEQ ID NO: 9 - RNA, 36 nt, Artificial Sequence
  Description of Artificial Sequence Synthetic oligonucleotide
cuuguuguauauauccuuuuauagguguugaacaac

SEQ ID NO: 10 - RNA, 36 nt, Artificial Sequence
  Description of Artificial Sequence Synthetic oligonucleotide
cuuguuguauauauccuuuuauggguguauaacaac

SEQ ID NO: 11 - RNA, 37 nt, Artificial Sequence
  Description of Artificial Sequence Synthetic oligonucleotide
cuuguuguauauguccuuuuauagguauuugaacaac

SEQ ID NO: 12 - RNA, 36 nt, Artificial Sequence
  Description of Artificial Sequence Synthetic oligonucleotide
cuuguuguauaugucuuuuuauagguauugaacaac

SEQ ID NO: 13 - RNA, 36 nt, Artificial Sequence
  Description of Artificial Sequence Synthetic oligonucleotide
cuuguuguguauauccuuuuauagguauugaacaac

SEQ ID NO: 14 - RNA, 12 nt, Artificial Sequence
  Description of Artificial Sequence Synthetic oligonucleotide
cuuguuguruau

SEQ ID NO: 15 - RNA, 12 nt, Artificial Sequence
  Description of Artificial Sequence Synthetic oligonucleotide
uuuuaurgruru

SEQ ID NO: 16 - DNA, 20 bp, Unknown
  Description of Unknown EMX1 Mammalian target sequence
ccgccgcttcctgagccatc

SEQ ID NO: 17 - RNA, 56 nt, Artificial Sequence
  Description of Artificial Sequence Synthetic oligonucleotide
cuuguuguauauguccuuuuauagguauuaaacaacccgccgcuuccugagccauc

SEQ ID NO: 18 - DNA, 20 bp, Unknown
  Description of Unknown VEGFA Mammalian target sequence
tccagaccaccaatgggcac

SEQ ID NO: 19 - RNA, 56 nt, Artificial Sequence
  Description of Artificial Sequence Synthetic oligonucleotide
cuuguuguauauguccuuuuauagguauuaaacaacuccagaccaccaaugggcac

SEQ ID NO: 20 - DNA, 20 bp, Unknown
  Description of Unknown AAVS1 Mammalian target sequence
tgagaatggtgcgtcctagg

SEQ ID NO: 21 - DNA, 2235 bp, Unknown
  Description of Unknown parent sequence
atgattaaatccatccagcttaaagtgaaaggcgaatgccccatcaccaaagatgttattaacgagtacaaggaatactataataactgcagcgattggataaaaaacaatcttacctcgatcactattggcgagatggccaagttcctgcagagcctgtctgataaggaggttgcttacatttccatgggcctgagcgacgagtggaaagacaagcccttgtaccacctgttcactaagaaataccacactaagaatgctgacaatctgctctactactacatcaaggagaaaaacttggatggctacaaggggaacacgcttaacatttctaacacctcatttcgtcagttcgggtatttcaagctcgtggtgtccaactatcgcactaagattcggacactcaactgcaagattaaacgaaagaagatcgacgcagattctacgagcgaggatattgaaatgcaggtcatgtatgaaatcatcaagtactctctgaataagaagtctgactgggacaattttatcagctacatcgaaaacgtagagaaccctaacatagacaatatcaacaggtacaaactgcttagggaatgcttctgtgaaaacgagaatatgataaaaaacaagctggagctgctgagcgtggagcaattaaagaagtttggagggtgtatcatgaaaccccacattaactcgatgacaattaatattcaagacttcaagatagaggagaaagaaaacagcttgggctttattctccatctccctctgaataaaaaacagtaccagatcgagctattgggaaatagacagattaagaaagggaccaaggaaattcacgaaactctcgtcgatatcacaaacacccatggagagaacatcgtgttcacaatcaagaacgataatctgtacatagtgtttagttatgagagcgagttcgagaaggaagaggtcaactttgccaagactgttgggcttgacgtcaatttcaaacacgcgttcttcgtgacaagcgaaaaggacaactgccatctcgatggctacattaacctatataagtatttgctcgaacacgacgagtttacaaacctgctgacagaagatgagagaaaggactacgaagaactcagtaaggtcgttacgttctgtccatttgaaaatcagctgctattcgcccggtacaataaaatgtccaaattctgtaagaaggaacaggtattgtctaaactgctgtatgccctgcagaaaaagttgaaagatgagaatcggaccaaagagtatatctatgtctcatgcgtgaacaagctaagagctaagtatgtttcctatttcatactgaaggagaagtactatgagaagcaaaaggagtacgacattgagatgggcttcgtcgatgactcaaccgaatctaaagaatccatggacaaaaggcgcacagagtatccatttagaaatacaccggtggctaacgaactcctgagtaaactcaacaatgttcaacaagatatcaacggttgtctgaagaatataattaattatatctataagatttttgaacagaatggctacaaggtggtcgcactggaaaacttagagaattccaactttgagaaaaagcaggtgcttcctacaatcaaatcacttctgaagtaccacaaacttgagaatcagaacgtaaatgatatcaaagccagtgataaagtcaaagaatacatcgagaatggttattatgaactgatgactaatgaaaataatgaaatagtggacgcaaaatatacggaaaagggtgccatgaaggtaaagaacgcaaacttttttaatttgatgatgaagtcactgcactttgcttctgtgaaggatgagtttgtcctgctgagcaacaacggaaagacacagattgcgctagtgccttcagagttcactagtcagatggacagtaccgaccattgcctctacatgaaaaaaaatgacaaagggaaactcgtgaaggctgacaagaaagaggtgcggaccaagcaagagcgccatatcaatggattaaacgccgattttaacgcggcaaataacatcaaatacatcgttgaaaatgaggtgtggaggggtatcttctgtacccgaccaaagaagactgagtacaacgtaccatccttagacaccaccaaaaaaggaccctccgccattctgaatatgttaaagaaaatcgaggccataaaagtgttggagaccgagaag

SEQ ID NO: 22 - DNA, 2235 bp, Unknown
  Description of Unknown parent sequence
atgatcaagagcattcagctgaaggtgaagggcgagtgccccatcaccaaggacgtgatcaacgagtacaaggagtactacaacaactgcagcgactggattaaaaacaatctcacaagcatcaccatcggcgagatggccaagttcctgcagagcctgagcgacaaagaggtggcctacatcagcatgggcctgagcgacgagtggaaggacaagcccctgtaccacctgttcaccaagaagtaccacaccaagaacgccgacaacctgctgtactactacatcaaggagaagaatctggatggctacaagggcaataccctgaacatcagcaacaccagcttcagacagttcggctacttcaagctggtggtgagcaactacagaaccaagatcagaaccctgaactgcaagatcaagagaaagaagatcgacgccgacagcacaagcgaggacatagagatgcaagttatgtacgagatcatcaagtacagccttaataaaaagagcgactgggacaacttcatcagctacatagaaaacgtggagaaccccaacatcgacaacatcaacagatacaagctgctgagagagtgcttctgcgagaacgagaacatgatcaaaaacaaactcgaactgctgagtgtagaacagctgaagaagttcggcggctgcatcatgaagccccacatcaacagcatgaccatcaacatccaagacttcaagatcgaggagaaggagaacagcctgggcttcatcctgcacctgcccttaaacaagaagcagtatcagatcgagctgctgggcaacagacagatcaagaagggcaccaaggagatccacgagaccctggtggacatcaccaacacccacggcgagaatatcgttttcactatcaagaacgacaacctgtacatcgtgtttagctatgagagcgagttcgagaaggaagaggtgaacttcgccaagaccgtgggcctggacgtgaacttcaagcacgccttcttcgtgacaagcgagaaggacaactgccacctggacggctacatcaatctgtacaagtacctgctggagcacgacgagttcaccaacctgctgaccgaggacgagagaaaggactacgaggagctgagcaaggtggtgaccttctgccccttcgagaatcagctgctgttcgctagatacaacaagatgagcaagttctgcaagaaggagcaagtgctgagcaaactgctgtacgccctgcagaagaagctgaaggacgagaacagaaccaaggagtatatctacgtgagctgcgtgaacaagctgagagccaagtacgtgagctacttcatcctgaaggagaagtactacgagaagcagaaggagtacgacatcgagatgggttttgtggacgacagcaccgagagcaaggagagcatggacaagagaagaaccgagtaccccttcagaaacacccccgtggccaacgaattactgtctaaactgaataacgtgcagcaagacatcaacggctgcctgaagaacataatcaactacatctacaagatcttcgagcagaacggctacaaggtggtagccctggagaacctggagaacagcaacttcgagaagaagcaagtgctgcccaccatcaagagcctgctgaagtaccacaagctggagaatcagaacgtgaacgacatcaaggctagcgacaaggtgaaggagtacatcgagaacggatactacgagctgatgaccaacgagaacaacgagatcgtggacgccaagtacaccgagaagggcgccatgaaggtgaagaacgccaacttcttcaacctgatgatgaagagcctgcacttcgctagcgtgaaggacgagttcgtgctgctgtcgaacaacggcaagacacagatcgccctggtgcctagcgagttcacatctcagatggacagcaccgaccactgcctgtacatgaagaagaacgacaagggcaagctggtgaaggccgacaagaaagaggtgagaaccaagcaagagagacacatcaacggcctgaacgccgacttcaacgccgccaacaacatcaagtacatcgtggagaacgaggtgtggagaggcatcttctgcacaagacccaagaagaccgagtacaacgtgcctagcctggacaccaccaagaagggccctagcgccatcctgaacatgctgaagaagatcgaggccatcaaggtgctggagaccgagaag

SEQ ID NO: 23 - DNA, 2235 bp, Unknown
  Description of Unknown parent sequence
atgatcaagagcatccagctgaaggtgaagggcgagtgccccatcaccaaggacgtgatcaacgagtacaaggagtactacaacaactgcagcgactggatcaagaacaacctgaccagcatcaccatcggcgagatggccaagttcctgcagagcctgagcgacaaggaggtggcctacatcagcatgggcctgagcgacgagtggaaggacaagcccctgtaccacctgttcaccaagaagtaccacaccaagaacgccgacaacctgctgtactactacatcaaggagaagaacctggacggctacaagggcaacaccctgaacatcagcaacaccagcttccggcagttcggctacttcaagctggtggtgagcaactaccggaccaagatccggaccctgaactgcaagatcaagcggaagaagatcgacgccgacagcaccagcgaggacatcgagatgcaggtgatgtacgagatcatcaagtacagcctgaacaagaagagcgactgggacaacttcatcagctacatcgagaacgtggagaaccccaacatcgacaacatcaaccggtacaagctgctgcgggagtgcttctgcgagaacgagaacatgatcaagaacaagctggagctgctgagcgtggagcagctgaagaagttcggcggctgcatcatgaagccccacatcaacagcatgaccatcaacatccaggacttcaagatcgaggagaaggagaacagcctgggcttcatcctgcacctgcccctgaacaagaagcagtaccagatcgagctgctgggcaaccggcagatcaagaagggcaccaaggagatccacgagaccctggtggacatcaccaacacccacggcgagaacatcgtgttcaccatcaagaacgacaacctgtacatcgtgttcagctacgagagcgagttcgagaaggaggaggtgaacttcgccaagaccgtgggcctggacgtgaacttcaagcacgccttcttcgtgaccagcgagaaggacaactgccacctggacggctacatcaacctgtacaagtacctgctggagcacgacgagttcaccaacctgctgaccgaggacgagcggaaggactacgaggagctgagcaaggtggtgaccttctgccccttcgagaaccagctgctgttcgcccggtacaacaagatgagcaagttctgcaagaaggagcaggtgctgagcaagctgctgtacgccctgcagaagaagctgaaggacgagaaccggaccaaggagtacatctacgtgagctgcgtgaacaagctgcgggccaagtacgtgagctacttcatcctgaaggagaagtactacgagaagcagaaggagtacgacatcgagatgggcttcgtggacgacagcaccgagagcaaggagagcatggacaagcggcggaccgagtaccccttccggaacacccccgtggccaacgagctgctgagcaagctgaacaacgtgcagcaggacatcaacggctgcctgaagaacatcatcaactacatctacaagatcttcgagcagaacggctacaaggtggtggccctggagaacctggagaacagcaacttcgagaagaagcaggtgctgcccaccatcaagagcctgctgaagtaccacaagctggagaaccagaacgtgaacgacatcaaggccagcgacaaggtgaaggagtacatcgagaacggctactacgagctgatgaccaacgagaacaacgagatcgtggacgccaagtacaccgagaagggcgccatgaaggtgaagaacgccaacttcttcaacctgatgatgaagagcctgcacttcgccagcgtgaaggacgagttcgtgctgctgagcaacaacggcaagacccagatcgccctggtgcccagcgagttcaccagccagatggacagcaccgaccactgcctgtacatgaagaagaacgacaagggcaagctggtgaaggccgacaagaaggaggtgcggaccaagcaggagcggcacatcaacggcctgaacgccgacttcaacgccgccaacaacatcaagtacatcgtggagaacgaggtgtggcggggcatcttctgcacccggcccaagaagaccgagtacaacgtgcccagcctggacaccaccaagaagggccccagcgccatcctgaacatgctgaagaagatcgaggccatcaaggtgctggagaccgagaag

SEQ ID NO: 24 - DNA, 2235 bp, Unknown
  Description of Unknown parent sequence
atgattaaaagtatacagcttaaggtcaagggcgagtgcccgattaccaaagatgtaattaacgagtacaaggagtattacaataactgttctgactggatcaagaacaatctgacttcaattacgatcggtgaaatggccaaatttctccagtccttgagtgataaggaggtcgcatatatatccatgggactcagtgacgagtggaaggacaaacccctgtaccatctttttactaaaaaataccatacgaaaaacgcagacaacctgctgtactactatataaaagaaaaaaacctggacggttacaagggcaacaccctcaatattagcaacactagtttccgacaattcgggtacttcaagctggtcgtgagcaactatcggaccaaaatcaggactttgaattgtaagataaagagaaagaagatagacgcagattctactagtgaggatatcgagatgcaggtaatgtacgagatcattaagtattcactgaacaagaagagcgattgggataacttcatatcatacatcgaaaacgttgaaaatccaaacatcgacaatattaatagatacaaactgttgagagaatgcttctgcgaaaacgaaaatatgataaaaaacaaactcgaattgttgtcagttgaacagctgaagaaattcggagggtgcataatgaagcctcacataaactcaatgacaataaacatccaagacttcaagatagaagagaaggagaacagtttgggttttattcttcacctgcctttgaacaagaagcaataccaaatcgagctgctcggaaataggcagataaaaaaggggacaaaagaaatacacgaaacccttgttgacattacgaacacacacggggaaaatatcgtgttcacaattaaaaatgacaacctttacattgtattttcttatgaatcagagttcgaaaaggaagaagtgaactttgccaagaccgttggtttggacgtcaactttaaacacgccttcttcgttacatcagaaaaggataactgtcatcttgatggatacataaacctctacaagtatcttcttgaacacgacgaattcacaaacctgcttactgaagacgagcgaaaggattacgaggagttgtcaaaggtagtcacattctgtccattcgaaaatcagctgttgtttgccaggtacaataagatgtctaaattctgtaaaaaagaacaagtcctcagcaaactgctgtatgcactgcaaaagaaacttaaggacgaaaataggactaaggagtatatatacgtttcatgcgttaataaactccgggcgaaatatgtgagttattttattctgaaggagaagtattatgagaagcagaaagagtacgacatagaaatgggatttgtggatgacagtacggagagcaaagaaagcatggacaaaagaaggaccgaatatccatttcgaaatactccagtcgcgaatgagctgctgagcaaacttaacaatgtccagcaggacattaacggttgcctgaagaacataatcaactacatatataagatatttgagcaaaacggatacaaagtggttgcacttgaaaacctcgagaattcaaatttcgaaaagaagcaagttttgcccacgattaaaagtctcttgaaataccataagctcgaaaatcagaatgtgaacgatatcaaggcctcagataaggtcaaggagtacatcgaaaatggatattacgagctgatgacgaacgagaataacgaaatcgtcgatgcgaagtacacagaaaagggggctatgaaggtgaaaaacgccaatttttttaatttgatgatgaagtccttgcatttcgcctcagtcaaagatgagtttgttctcctgagtaataatgggaaaacacagatagccttggttccttcagagttcacgtctcagatggactcaactgatcattgtctttatatgaagaaaaatgataaagggaaactggtcaaggccgataagaaagaggtgcgcacgaaacaagaaagacacatcaacggcctcaacgccgattttaacgcagctaacaatattaaatatatcgtagagaatgaggtctggaggggcattttttgcacccgacccaagaagactgaatacaatgtccctagtctcgatacgaccaaaaaggggccatcagctatactgaatatgttgaaaaagattgaggcgattaaagtcctggaaaccgagaaa

SEQ ID NO: 25 - DNA, 2235 bp, Unknown
  Description of Unknown parent sequence
atgatcaagagcatccagctgaaggtgaagggagaatgtcctatcacaaaggacgtgatcaacgagtacaaggagtactacaacaactgcagcgattggatcaagaacaacctgaccagcatcaccatcggcgagatggccaagttcctgcagtctctgtccgataaggaagtcgcctacatcagcatgggcttgagcgacgaatggaaggacaaacccctgtaccatctgttcactaagaagtaccacaccaagaacgccgataacctgctgtactactacatcaaggaaaagaacctggacggctacaagggcaataccctgaacatcagtaacaccagcttccggcagtttggctattttaaactggtggtcagcaactacagaaccaagatcagaacactgaactgcaagatcaagaggaagaaaatcgacgccgactccaccagcgaagatatagagatgcaggtgatgtacgagatcattaagtactccctgaataagaagtctgactgggacaacttcatcagctacatcgagaacgtggaaaaccccaacattgacaatatcaacagatacaagctgctgcgggaatgcttctgcgagaatgaaaacatgatcaagaacaagctggagcttctgagcgtggagcagctgaagaaattcggcggatgtatcatgaagccacacatcaatagcatgaccatcaacatccaggacttcaaaattgaagaaaaggagaatagcctaggcttcatcctgcacctgcctctgaacaagaaacagtaccagatcgagctgctgggcaaccggcaaatcaagaagggcaccaaggagatccacgagacactggtcgacatcacaaacacacacggcgaaaacatcgtgttcaccatcaagaacgacaacctgtacatcgtgttcagctacgagtctgaattcgagaaggaagaggtcaacttcgctaagacagtgggcctggacgtgaacttcaagcacgccttcttcgtgaccagcgagaaagacaactgtcacctggacgggtacatcaacctgtacaagtacctgctggaacacgacgagttcaccaacctcctgaccgaagatgaacggaaggattacgaggagctgtctaaggtggtgacattctgccctttcgagaaccagctgctcttcgccagatataacaagatgagcaagttttgtaaaaaggagcaggtgctcagcaagctactgtacgccctgcagaagaagctgaaggacgagaacagaaccaaggaatacatctacgtgagctgcgtgaacaagctgagagccaagtacgtgtcctatttcatcctgaaggaaaaatactacgagaaacagaaagagtacgacatcgagatgggatttgtggacgacagcaccgaaagcaaggaatctatggacaagcgcagaaccgagtatccatttagaaacacccctgtggccaatgagctgctgtccaaactgaacaacgtgcagcaggatatcaatggctgcctgaaaaacatcatcaactacatttacaagatctttgagcaaaacggctacaaagtggtggccctggagaacctggaaaactccaacttcgagaagaagcaagtgctgcccacaatcaagagcctgctgaagtaccataagctggaaaatcagaatgtgaacgacataaaggcctctgataaggtgaaggaatacatcgaaaatggctattacgagctgatgaccaacgaaaataacgagattgtggacgctaaatacaccgagaagggcgctatgaaagtgaaaaacgctaacttttttaacctgatgatgaagagcctgcacttcgccagcgtgaaggacgagttcgtgctgctgagcaacaacggcaagacacagatcgccctggtgcccagcgagttcaccagtcagatggattctacagatcactgcctgtacatgaaaaagaatgataagggaaagttagtgaaagccgataagaaggaggtgcggaccaaacaggagagacacatcaacggcctgaacgctgacttcaacgccgccaacaacatcaaatacatcgttgagaatgaggtgtggcggggcatcttctgcaccagacctaagaaaacagagtataatgtgcctagcctggacaccaccaagaagggtcctagcgccatcctgaacatgctgaagaagatcgaggccatcaaggttctggaaaccgagaag

SEQ ID NO: 26 - DNA, 2235 bp, Unknown
  Description of Unknown parent sequence
atgatcaaatcaattcagcttaaggtgaagggcgagtgtcccattactaaagacgtcattaacgaatacaaggagtattacaataactgtagcgactggatcaagaacaatttgacatctatcacaattggggagatggccaaatttttgcagagccttagcgacaaagaggtcgcctacatctctatggggctctccgacgagtggaaggacaagccgttgtaccacctgttcacaaaaaagtatcacacaaagaatgcagacaatttgctgtactactacattaaggagaaaaaccttgatggctataaaggaaacaccctcaacatctctaacacctcttttagacagttcggctactttaagctggtggtgagcaactatagaaccaagattaggactctgaattgtaagatcaaaaggaagaaaatcgacgctgattctacctctgaagatattgagatgcaagtcatgtatgagatcatcaagtattcactgaacaagaagagtgattgggacaactttatttcctacatagagaacgtggagaatcccaatatcgataatataaaccgatataagttgctgcgggagtgcttttgtgaaaatgagaacatgattaaaaacaagttggaactcttgtcagtagaacagcttaaaaagttcggcggctgcatcatgaagcctcatatcaacagcatgacaattaatatccaagattttaagatcgaggagaaggaaaacagtttggggtttattttgcaccttccactcaataagaaacagtaccagatcgagcttctggggaatcggcagattaagaaagggaccaaagagatacatgaaactctggttgatatcacaaatacccatggcgaaaatattgtctttaccatcaagaatgacaacctgtatatcgtgttttcttatgagtccgaattcgagaaggaagaggtgaactttgccaagacagtggggttggatgtcaattttaagcatgccttctttgttacctctgaaaaagataactgccatctggatggatacatcaatctctataagtatctgcttgagcacgatgagttcacaaatctgctgaccgaggacgagagaaaggactacgaagaattgtccaaggtggttactttctgcccattcgaaaatcagctgctgttcgcaagatacaacaaaatgtctaaattctgtaaaaaagagcaggtcctgtctaaacttctgtatgctctgcagaaaaaactcaaagacgaaaaccgcacaaaagagtacatttacgtaagctgcgtcaataaattgagggccaagtatgtttcttatttcatccttaaagagaaatactacgagaagcagaaggagtacgacatagagatggggtttgtagacgattctacggagtctaaggaatctatggacaaacggcgcacagagtacccctttaggaatacccctgtcgctaatgagttgctctctaagctgaacaatgtccaacaggatattaatggctgcttgaagaacataataaattatatttataaaatctttgagcagaacggctacaaagttgttgctctggagaaccttgagaacagcaactttgaaaagaagcaggtactgcctactattaagtctctgctgaaatatcataagctggaaaaccaaaatgtcaacgacatcaaggcctccgacaaagtgaaagagtacatcgagaacggatactacgagctgatgactaatgagaataacgagattgtcgacgcgaaatataccgaaaagggcgctatgaaggtgaagaatgctaatttcttcaacttgatgatgaagagcctccattttgcttctgtgaaggatgagtttgtgctgttgagtaacaacgggaagacccagatcgcactggtcccgtccgagtttacctcccagatggactctacagatcactgcttgtacatgaagaaaaacgataaaggtaagttggtgaaagcagataagaaagaggtgcgaacaaagcaggagagacatatcaacggactgaacgcagattttaacgctgctaataatattaaatatattgttgagaacgaagtgtggcggggaattttctgcaccagaccgaaaaaaacagaatataatgtcccgtccctcgacactactaaaaaaggcccttctgcgatcctgaacatgcttaaaaagattgaagcgatcaaggtgttggagactgagaag

SEQ ID NO: 27 - DNA, 2235 bp, Unknown
  Description of Unknown parent sequence
atgatcaagagcattcagctgaaagtgaagggcgagtgtcccatcactaaggacgttatcaacgagtacaaggagtactataacaactgctctgactggattaagaataacctgaccagtatcaccatcggcgagatggccaagttcctgcagagcctgagcgacaaggaggtggcctacatcagtatggggctgtccgatgagtggaaagacaagccactgtatcatctgttcaccaaaaagtaccacaccaaaaatgccgataacctgctgtactattatatcaaagaaaaaaacctggacgggtacaaaggcaacaccctgaacatcagcaatacaagtttccgccagtttggatacttcaagctggtcgtgtcaaattacagaaccaaaatcagaaccctgaactgcaagatcaaaaggaagaagatcgacgctgacagcaccagcgaggacattgagatgcaggtgatgtacgagattattaaatactccctgaacaagaagagtgattgggacaatttcatttcctacatcgagaatgtggagaaccctaacatcgataacataaatcggtacaaactgctgagagagtgcttttgcgagaacgaaaatatgataaagaacaagctggagctgctcagcgtggaacagctgaaaaaatttggaggctgcattatgaagccccacatcaactcaatgactatcaatatccaggacttcaagatcgaagagaaggagaactccctgggcttcattctgcatctgccactgaataagaagcagtatcagatcgaactgctgggaaataggcagatcaagaaggggaccaaagagatccacgaaactctggtggacattactaatactcacggggagaacattgtgttcaccattaagaacgataatctgtacatcgtgttcagctatgaaagcgagtttgagaaggaagaggtgaacttcgccaagaccgtcgggctggatgtgaattttaagcacgccttctttgtgaccagcgaaaaggataactgccacctggacggatacatcaatctgtacaagtatctgctggagcacgacgaatttaccaatctgctgaccgaggacgagaggaaggactacgaagagctctccaaggtggtgacattttgtccattcgagaaccagctgctgttcgccagatacaacaagatgtctaaattttgcaaaaaagaacaggtcctgagcaagctgctgtacgccctgcagaagaaactgaaggacgagaacagaactaaggagtacatctatgtgagctgtgtgaataaactgagagcaaaatacgtgtcttactttattctgaaggagaagtattacgagaagcagaaggaatacgacatcgagatgggcttcgtggacgattccacagagagcaaggaaagcatggataaaaggcgcactgaataccccttcagaaacacacctgtggccaatgagctgctgtccaagctgaacaacgtgcagcaggacatcaacggctgcctgaagaacatcatcaactacatttataagatcttcgagcagaatggctacaaagtggtggcactggagaacctggagaactccaattttgaaaagaaacaggtgctgcccaccattaagagcctgcttaaataccataagctggagaaccagaacgtgaatgatatcaaagccagcgataaggtcaaagagtacatcgagaacggatattatgagctgatgaccaacgagaacaatgagatcgtggacgccaaatataccgagaagggcgccatgaaggtgaagaacgccaacttctttaacctgatgatgaagtcccttcactttgcctccgtgaaggatgagttcgtgctgctgagcaacaacggcaagacccagatcgccctggtgccctctgaattcaccagccagatggattcaaccgaccactgtctctacatgaagaagaacgacaagggcaagctggtgaaggccgacaagaaagaggtgcgcacaaagcaggagcggcacatcaatggcctgaacgctgatttcaacgccgcaaacaacattaagtacatcgtggagaatgaggtgtggagaggtattttctgcacccgccccaagaagaccgagtataatgtgccctctctggacaccaccaaaaaaggcccaagcgctatcctgaatatgttgaagaaaatcgaggccatcaaggtgctggaaaccgagaag

SEQ ID NO: 28 - RNA, 56 nt, Artificial Sequence
  Description of Artificial Sequence Synthetic oligonucleotide
cuuguuguauauguccuuuuauagguauuaaacaacugagaauggugcguccuagg